(19) United States
(12) Patent Application Publication
(10) Pub. No.: US 2024/0094207 A1
(43) Pub. Date: Mar. 21, 2024

(54) SEROLOGICAL BIOMARKERS FOR EARLY DIAGNOSIS OF LUNG CANCER

(71) Applicants: THE JOHNS HOPKINS UNIVERSITY, Baltimore, MD (US); CDI LABORATORIES, INC., Mayaguez, PR (US)

(72) Inventors: Ignacio Pino, Mayaguez, PR (US); Heng Zhu, Baltimore, MD (US); Jiang Qian, Baltimore, MD (US); Yi Huang, Mayaguez, PR (US)

(21) Appl. No.: 18/448,425

(22) Filed: Aug. 11, 2023

Related U.S. Application Data
(63) Continuation of application No. 16/499,950, filed on Oct. 1, 2019, now abandoned.
(60) Provisional application No. 62/480,906, filed on Apr. 3, 2017.

(51) Int. Cl. G01N 33/574; G01N 33/564; G16H 50/20

(52) U.S. Cl. CPC G01N 33/57423; G01N 33/564; G16H 50/20

(57) ABSTRACT

Biomarkers to screen for, identify, and/or characterize lung cancer in a subject are disclosed. Also disclosed herein are methods for distinguishing lung cancer from another disease. Also disclosed herein are methods for detecting metastasis of a lung cancer in a subject. Also disclosed herein are substrates, arrays, and reagents for use in the methods, and methods of their preparation.

### CROSS-REFERENCE

This application is a continuation of U.S. application Ser. No. 16/499,950, filed Oct. 1, 2019, which is a national phase application under 35 U.S.C. § 371 of PCT International Application No.: PCT/US2018/025726, filed on Apr. 2, 2018, which claims priority to U.S. Provisional Application No. 62/480,906, filed on Apr. 3, 2017, which is hereby incorporated by reference in its entirety.

### SEQUENCE LISTING

The text of the computer readable sequence listing filed herewith, titled “JHU-40000-303_SQL”, created Aug. 11, 2023, having a file size of 112,966 bytes, is hereby incorporated by reference in its entirety.

### BACKGROUND

Lung cancer (LC) remains the leading cause of mortality from malignant tumors worldwide. According to the World Health Organization (WHO), among the 8.2 million cancer-related deaths in 2012, LC caused 1.59 million deaths worldwide. In China, LC alone is responsible for the mortality of 42.05 per 100,000 persons. LC can be classified into two main types: small-cell lung cancer (SCLC) and non-small-cell lung cancer (NSCLC). Approximately 80% of diagnosed LC is NSCLC. The three main subclasses of NSCLC are adenocarcinoma, squamous cell carcinoma and large cell carcinoma, which account for 40%, 30%, and 9% of LC, respectively. The degree of spread of LC from its original source is assessed by LC staging, which often affects the prognosis and treatment of LC. The evaluation of NSCLC staging is based on the size of the primary tumor, lymph node involvement, and distant metastasis, a.k.a. TNM classification. SCLC, on the other hand, has traditionally been classified as limited stage or extensive stage.

Regardless of the great progress achieved in recent years against LC, surgery is still the preferred method in the treatment of NSCLC patients in early stages (e.g., I-II LC), and when surgery is performed, there is a 70% one-year survival rate if the diagnosis is made at the earliest stage. Unfortunately, the majority of LC patients are found in late stages at the time of diagnosis. For example, 60% of SCLC patients are diagnosed at the most advanced stage. Therefore, identification of non-invasive serological biomarkers for early stage LC diagnosis will greatly benefit intervention and prevention of LC.

### SUMMARY

Disclosed herein are methods for contacting a sample from a subject with a plurality of isolated and purified tumor-associated proteins or fragments thereof. In some embodiments, at least three of the plurality of isolated and purified tumor-associated proteins or fragments thereof can be selected from the group consisting of ethylmalonic encephalopathy protein 1 (ETHE1), tumor protein p53 (p53), Cancer/Testis Antigen 1A (CTAG1A), C1q And Tumor Necrosis Factor Related Protein 1 (C1QTNF1), Testis Expressed 264 (TEX264), Claudin 2 (CLDN2), Neuron Specific Gene Family Member 1 (NSG1), GTPase HRas (HRas), Cytoskeleton Associated Protein 2 (CKAP2), Dipeptidyl peptidase 4 (DPP4), Calcium-binding protein 39 (CAB39), Centromere protein X (STRA13), an antigenic fragment of any of the above, and a polypeptide having at least 80 percent sequence homology with a sequence selected from SEQ ID NOs: 1-12 or an antigenic fragment thereof. In some embodiments, the method can further comprise detecting a binding of at least one of the plurality of isolated and purified tumor-associated proteins or fragments thereof to a moiety. In some embodiments the subject can have or can be suspected of having lung cancer. In some embodiments, the at least one of the plurality of isolated and purified tumor associated proteins or fragments thereof can comprise ETHE1, a fragment thereof, or a polypeptide having at least 80 percent sequence homology with the sequence of SEQ ID NO: 1 or an antigenic fragment thereof. In some embodiments, the plurality of isolated and purified tumor-associated proteins or fragments thereof can comprise less than 10,000 sequentially unique purified tumor-associated proteins or fragments thereof. In some embodiments, sequence homology can be determined by a sequence alignment performed using BLAST. In some embodiments, the plurality of isolated and purified tumor-associated proteins or fragments thereof can comprise less than 100 sequentially unique purified tumor-associated proteins or fragments thereof. In some embodiments, the plurality of isolated and purified tumor-associated proteins or fragments thereof can comprise less than 10 sequentially unique purified tumor-associated proteins or fragments thereof. In some embodiments, the method can further comprise further comprising characterizing the subject as having an increased probability of having lung cancer. In some embodiments, the characterizing can be performed with a sensitivity of at least about 52%. In some embodiments, moiety can comprise an autoantibody. In some embodiments, the characterizing can be performed with a sensitivity of at least about 56%. In some embodiments, the characterizing can be performed with a specificity of at least about 91%. In some embodiments, the characterizing can be performed with a specificity of at least about 92%. In some embodiments, the moiety can bind to at least a portion of an antigenic sequence as defined in SEQ ID NO: 1. In some embodiments, the moiety can bind to at least a portion of an antigenic sequence having at least 80% sequence homology to SEQ ID NO: 1. In some embodiments, the detecting can comprise detecting a signal. In some embodiments, the signal can be detected by or after associating the moiety with a probe. In some embodiments, the probe can be directly or indirectly associated with the moiety. In some embodiments, the probe can be an anti-immunoglobulin antibody. In some embodiments, the probe can comprise or can be associated with a chromophore. In some embodiments, the chromophore can be a fluorescent marker. In some embodiments, the signal can have a Z score greater than a cutoff value of 1. In some embodiments, the signal can be detected if the signal is at least about 2 standard deviations greater than a reference signal. In some embodiments, the reference signal can be detected after contacting a second sample from a second subject with a plurality of tumor-associated proteins or fragments thereof. In some embodiments, the second subject can be a non-diseased subject. In some embodiments, the detecting can be carried out using a computer. In some embodiments, the computer can be a portable device. In some embodiments, the method can be a method of screening for the presence or absence of lung cancer. In some embodiments, the method can be a method of distinguishing lung cancer from a second disease. In some embodiments, the detecting can be indicative of the subject having lung cancer and not the second disease. In some embodiments, the second disease can be benign lung lesions (LBL), pneumonia, chronic obstructive pulmonary disease (COPD), pulmonary tuberculosis, or a second cancer. In some embodiments, the second disease can be a second cancer. In some embodiments, the second cancer can be rectal cancer, liver cancer, cervical cancer, esophagus cancer, or gastric cancer. In some embodiments, the lung cancer can be small-cell lung cancer (SCLC) or non-small cell lung cancer (NSCLC). In some embodiments, the lung cancer can be NSCLC. In some embodiments, the NSCLC can comprise adenocarcinoma, squamous cell carcinoma, or large cell carcinoma. In some embodiments, the lung cancer can be an early stage lung cancer. In some embodiments, the lung cancer can be a late stage lung cancer. In some embodiments, the subject may not be diabetic. In some embodiments, the subject may have been previously diagnosed with a cancer. In some embodiments, the method can further comprise detecting metastatic cancer in the subject. In some embodiments, the method can be a confirmatory test. In some methods, the subject can be human. In some embodiments, the sample can comprise a body fluid. In some embodiments, the body fluid can comprise blood or a fraction thereof. In some embodiments, the blood fraction can comprise plasma or serum. In some embodiments, the method can further comprise selecting a therapeutic based on the result of the method. In some embodiments, the methd can further comprise a second diagnostic evaluation. In some embodiments, the subject can be a past, current, or future smoker. In some embodiments, the subject can have a family history of lung cancer. In some embodiments, the subject can be genetically predisposed to lung cancer. In some embodiments, the method can further comprise treating a subject. In some embodiments, the treatment can comprise one or more of: surgery, chemotherapy, radiation therapy, immunotherapy, targeted therapy, hormone therapy, stem cell transplant, and precision medicine. In some embodiments, the treatment comprises surgery, wherein the surgery comprises cryosurgery, laser therapy, hyperthermia, photodynamic therapy, open surgery, or laparoscopy. In some embodiments, the treatment can comprise chemotherapy, wherein the chemotherapy can comprise an alkylating agent, an anti-metabolite, an anti-tumor antibiotic, a topoisomerase inhibitor, a mitotic inhibitor, a corticosteroid, a proteasome inhibitor, or a kinase inhibitor. In some embodiments, the treatment can comprise radiation therapy, wherein the radiation therapy can comprise external beam radiation therapy or internal radiation therapy. In some embodiments, the treatment can comprise immunotherapy, wherein the immunotherapy comprises a monoclonal antibody, adoptive cell transfer, a cytokine, a treatment vaccine, or Bacillus Calmette-Guérin (BCG) therapy. In some embodiments, the treatment can comprise targeted therapy, wherein the targeted therapy comprises a small molecule drug or a monoclonal antibody. In some embodiments, the treatment can comprise hormone therapy, wherein the hormone therapy can comprise an estrogen receptor blocker, an aromatase inhibitor, a luteinizing hormone blocker, an antiandrogen, a gonadotropin releasing hormone blocker, or a progesterone receptor blocker. In some embodiments, the treatment can comprise stem cell transplant, wherein the stem cell transplant comprises autologous stem cell transplant, allogeneic stem cell transplant, or syngeneic stem cell transplant. In some embodiments, the contacting can be performed on a solid support. In some embodiments, the solid support can be an array. In some embodiments, the array can comprise a substrate. In some embodiments, the substrate can comprise an epoxy resin. In some embodiments, the plurality of tumor-associated proteins or fragments thereof can be covalently associated with the solid support. In some embodiments, the plurality of tumor-associated proteins or fragments thereof can be non-covalently associated with the solid support. In some embodiments, the solid support can further comprise an affinity ligand. In some embodiments, the affinity ligand can comprise an antigen, an antibody, an antibody fragment, glutathione, calmodulin, biotin, streptavidin, streptactin, amylose, or a metal chelate. In some embodiments, the metal chelate can comprise nickel, cobalt, zinc, mercury, or iron chelate. In some embodiments, the affinity ligand can at least partially coat a surface of the solid support. In some embodiments, the plurality of tumor-associated proteins or fragments thereof can comprise an affinity tag or can be associated with an affinity tag. In some embodiments, the affinity tag can comprise an antigen tag, an antibody tag, an antibody fragment tag, a calmodulin tag, a glutathione S-transferase (GST) tag, a histidine (His) tag, a streptavidin tag, an avidin tag, a maltose-binding protein tag, or a Flag tag. In some embodiments, an affinity tag can be coupled to the solid support covalently. In some embodiments, an affinity tag can be coupled to the solid support non-covalently. In some embodiments, an affinity tag can be coupled to the solid support by a linker. In some embodiments, an affinity ligand can be between the solid support and the plurality of tumor-associated proteins or fragments thereof. In some embodiments, the plurality of tumor-associated proteins or fragments thereof can be coupled to an affinity ligand covalently. In some embodiments, the plurality of tumor-associated proteins or fragments thereof can be coupled to an affinity ligand non-covalently. In some embodiments, the affinity ligand is between the solid support and the affinity tag. In some embodiments, the affinity tag can be coupled to the affinity ligand covalently. In some embodiments, the affinity tag can be coupled to the affinity ligand non-covalently. In some embodiments, the methods can further comprise repeating at different time points the steps of: (a) contacting a sample from a subject with a plurality of isolated and purified tumor-associated proteins or fragments thereof, wherein at least three of the plurality of isolated and purified tumor-associated proteins or fragments thereof are selected from the group consisting of ethylmalonic encephalopathy protein 1 (ETHE1), tumor protein p53 (p53), Cancer/Testis Antigen 1A (CTAG1A), C1q And Tumor Necrosis Factor Related Protein 1 (C1QTNF1), Testis Expressed 264 (TEX264), Claudin 2 (CLDN2), Neuron Specific Gene Family Member 1 (NSG1), GTPase HRas (HRas), Cytoskeleton Associated Protein 2 (CKAP2), Dipeptidyl peptidase 4 (DPP4), Calcium-binding protein 39 (CAB39), Centromere protein X (STRA13), an antigenic fragment of any of the above, and a polypeptide having at least 80 percent sequence homology with a sequence selected from SEQ ID NOs: 1-12 or an antigenic fragment thereof, as determined by a sequence alignment performed using BLAST; and; and (b) detecting a binding of at least one of the plurality of isolated and purified tumor-associated proteins or fragments thereof to a moiety. In some embodiments, the different time points can be within 1 week, 2 weeks, 1 month, 2 months, 3 months, 6 months, 8 months or 1 year. In some embodiments, the repeating (a) and (b) can be performed following administration of a treatment to the subject. In some embodiments, the detecting can be determinative of the subject's response to a treatment. In some embodiments, the detecting can be determinative at least in part for whether the subject is eligible for a clinical trial. In some embodiments, the detecting can determine a likelihood of the subject having an adverse reaction to a treatment. In some embodiments, the method can further comprise communicating the detecting via a communication medium. In some embodiments, the communication medium can comprise an electronic medium. In some embodiments, the electronic medium can comprise a device comprising a processor or a microprocessor. In some embodiments, one or more tumor-associated proteins or fragments thereof can comprise less than about 10,000 sequentially unique proteins or fragments thereof. In some embodiments, a moiety can be a peptide.

Also disclosed herein are methods for characterizing a subject as having a disease or condition. In some embodiments, a method can comprise contacting a sample from a subject with one or more proteins or fragments thereof. In some embodiments, a protein or fragments thereof can be isolated and purified. In some embodiments, a protein or fragments thereof can be a tumor-associated protein or fragment thereof. In some embodiments, a tumor-associated protein or fragment thereof can comprise ethylmalonic encephalopathy protein 1 (ETHE1) or an antigenic fragment thereof. In some embodiments, a tumor-associated protein or fragment thereof can comprise a polypeptide having at least 80 percent sequence homology with a sequence as shown in SEQ ID NO: 1. In some embodiments, a tumor-associated protein or fragment thereof can comprise a fragment of a polypeptide having at least 80% sequence homology with a sequence as shown in SEQ ID NO: 1. Also disclosed herein are methods further comprising detecting a binding of at least one or more tumor-associated proteins or fragments thereof to a moiety. Also disclosed herein are methods further comprising characterizing the subject as having an increase probability of having lung cancer, wherein the characterizing is performed with a sensitivity of at least about 52%. In some embodiments, moiety can comprise an autoantibody. In some embodiments, the characterizing can be performed with a sensitivity of at least about 56%. In some embodiments, the characterizing can be performed with a specificity of at least about 91%. In some embodiments, the characterizing can be performed with a specificity of at least about 92%. In some embodiments, one or more tumor-associated proteins or fragments thereof can further comprise tumor protein p53 (p53), Cancer/Testis Antigen 1A (CTAG1A), C1q And Tumor Necrosis Factor Related Protein 1 (C1QTNF1), Testis Expressed 264 (TEX264), Claudin 2 (CLDN2), Neuron Specific Gene Family Member 1 (NSG1), GTPase HRas (HRas), Cytoskeleton Associated Protein 2 (CKAP2), Dipeptidyl peptidase 4 (DPP4), Calcium-binding protein 39 (CAB39), Centromere protein X (STRA13), a fragment of any of the above, or a combination of any of the above. In some embodiments, the moiety can bind to at least a portion of an antigenic sequence as defined in SEQ ID NO: 1. In some embodiments, the moiety can bind to at least a portion of an antigenic sequence having at least 80% sequence homology to SEQ ID NO: 1. In some embodiments, the detecting can comprise detecting a signal. In some embodiments, the signal can be detected by or after associating the moiety with a probe. In some embodiments, the probe can be directly or indirectly associated with the moiety. In some embodiments, the probe can be an anti-immunoglobulin antibody. In some embodiments, the probe can comprise or can be associated with a chromophore. In some embodiments, the chromophore can be a fluorescent marker. In some embodiments, the signal can have a Z score greater than a cutoff value of 1. In some embodiments, the signal can be detected if the signal is at least about 2 standard deviations greater than a reference signal. In some embodiments, the reference signal can be detected after contacting a second sample from a second subject with one or more tumor-associated proteins or fragments thereof. In some embodiments, the second subject can be a non-diseased subject. In some embodiments, the detecting can be carried out using a computer. In some embodiments, the computer can be a portable device. In some embodiments, the method can be a method of screening for the presence or absence of lung cancer. In some embodiments, the method can be a method of distinguishing lung cancer from a second disease. In some embodiments, the detecting can be indicative of the subject having lung cancer and not the second disease. In some embodiments, the second disease can be benign lung lesions (LBL), pneumonia, chronic obstructive pulmonary disease (COPD), pulmonary tuberculosis, or a second cancer. In some embodiments, the second disease can be a second cancer. In some embodiments, the second cancer can be rectal cancer, liver cancer, cervical cancer, esophagus cancer, or gastric cancer. In some embodiments, the lung cancer can be small-cell lung cancer (SCLC) or non-small cell lung cancer (NSCLC). In some embodiments, the lung cancer can be NSCLC. In some embodiments, the NSCLC can comprise adenocarcinoma, squamous cell carcinoma, or large cell carcinoma. In some embodiments, the lung cancer can be an early stage lung cancer. In some embodiments, the lung cancer can be a late stage lung cancer. In some embodiments, the subject may not be diabetic. In some embodiments, the subject may have been previously diagnosed with a cancer. In some embodiments, the method can further comprise detecting metastatic cancer in the subject. In some embodiments, the method can be a confirmatory test. In some methods, the subject can be human. In some embodiments, the sample can comprise a body fluid. In some embodiments, the body fluid can comprise blood or a fraction thereof. In some embodiments, the blood fraction can comprise plasma or serum. In some embodiments, the method can further comprise selecting a therapeutic based on the result of the method. In some embodiments, the methd can further comprise a second diagnostic evaluation. In some embodiments, the subject can be a past, current, or future smoker. In some embodiments, the subject can have a family history of lung cancer. In some embodiments, the subject can be genetically predisposed to lung cancer. In some embodiments, the method can further comprise treating a subject. In some embodiments, the treatment can comprise one or more of: surgery, chemotherapy, radiation therapy, immunotherapy, targeted therapy, hormone therapy, stem cell transplant, and precision medicine. In some embodiments, the treatment comprises surgery, wherein the surgery comprises cryosurgery, laser therapy, hyperthermia, photodynamic therapy, open surgery, or laparoscopy. In some embodiments, the treatment can comprise chemotherapy, wherein the chemotherapy can comprise an alkylating agent, an anti-metabolite, an anti-tumor antibiotic, a topoisomerase inhibitor, a mitotic inhibitor, a corticosteroid, a proteasome inhibitor, or a kinase inhibitor. In some embodiments, the treatment can comprise radiation therapy, wherein the radiation therapy can comprise external beam radiation therapy or internal radiation therapy. In some embodiments, the treatment can comprise immunotherapy, wherein the immunotherapy comprises a monoclonal antibody, adoptive cell transfer, a cytokine, a treatment vaccine, or Bacillus Calmette-Guérin (BCG) therapy. In some embodiments, the treatment can comprise targeted therapy, wherein the targeted therapy comprises a small molecule drug or a monoclonal antibody. In some embodiments, the treatment can comprise hormone therapy, wherein the hormone therapy can comprise an estrogen receptor blocker, an aromatase inhibitor, a luteinizing hormone blocker, an antiandrogen, a gonadotropin releasing hormone blocker, or a progesterone receptor blocker. In some embodiments, the treatment can comprise stem cell transplant, wherein the stem cell transplant comprises autologous stem cell transplant, allogeneic stem cell transplant, or syngeneic stem cell transplant. In some embodiments, the contacting can be performed on a solid support. In some embodiments, the solid support can be an array. In some embodiments, the array can comprise a substrate. In some embodiments, the substrate can comprise an epoxy resin. In some embodiments, the one or more tumor-associated proteins or fragments thereof can be covalently associated with the solid support. In some embodiments, one or more tumor-associated proteins or fragments thereof can be non-covalently associated with the solid support. In some embodiments, the solid support can further comprise an affinity ligand. In some embodiments, the affinity ligand can comprise an antigen, an antibody, an antibody fragment, glutathione, calmodulin, biotin, streptavidin, streptactin, amylose, or a metal chelate. In some embodiments, the metal chelate can comprise nickel, cobalt, zinc, mercury, or iron chelate. In some embodiments, the affinity ligand can at least partially coat a surface of the solid support. In some embodiments, one or more tumor-associated proteins or fragments thereof can comprise an affinity tag or can be associated with an affinity tag. In some embodiments, the affinity tag can comprise an antigen tag, an antibody tag, an antibody fragment tag, a calmodulin tag, a glutathione S-transferase (GST) tag, a histidine (His) tag, a streptavidin tag, an avidin tag, a maltose-binding protein tag, or a Flag tag. In some embodiments, an affinity tag can be coupled to the solid support covalently. In some embodiments, an affinity tag can be coupled to the solid support non-covalently. In some embodiments, an affinity tag can be coupled to the solid support by a linker. In some embodiments, an affinity ligand can be between the solid support and one or more tumor-associated proteins or fragments thereof. In some embodiments, one or more tumor-associated proteins or fragments thereof can be coupled to an affinity ligand covalently. In some embodiments, one or more tumor-associated proteins or fragments thereof can be coupled to an affinity ligand non-covalently. In some embodiments, the affinity ligand is between the solid support and the affinity tag. In some embodiments, the affinity tag can be coupled to the affinity ligand covalently. In some embodiments, the affinity tag can be coupled to the affinity ligand non-covalently. In some embodiments, the methods can further comprise repeating at different time points the steps of: (a) contacting a sample from a subject with one or more tumor-associated proteins or fragments thereof, wherein the one or more tumor-associated proteins or fragments thereof can comprise ethylmalonic encephalopathy protein 1 (ETHE1) or a fragment having at least 80 percent sequence homology with a sequence as shown in SEQ ID NO: 1; and (b) detecting a binding of one or more tumor-associated proteins or fragments thereof to an moiety. In some embodiments, the different time points can be within 1 week, 2 weeks, 1 month, 2 months, 3 months, 6 months, 8 months or 1 year. In some embodiments, the repeating (a) and (b) can be performed following administration of a treatment to the subject. In some embodiments, the detecting can be determinative of the subject's response to a treatment. In some embodiments, the detecting can be determinative at least in part for whether the subject is eligible for a clinical trial. In some embodiments, the detecting can determine a likelihood of the subject having an adverse reaction to a treatment. In some embodiments, the method can further comprise communicating the detecting via a communication medium. In some embodiments, the communication medium can comprise an electronic medium. In some embodiments, the electronic medium can comprise a device comprising a processor or a microprocessor. In some embodiments, one or more tumor-associated proteins or fragments thereof can comprise less than about 10,000 sequentially unique proteins or fragments thereof. In some embodiments, a moiety can be a peptide.

Also disclosed herein are methods comprising contacting a sample from a subject with one or more isolated and purified tumor-associated proteins or fragments thereof. In some embodiments, the one or more tumor-associated proteins or fragments thereof can comprise at least one of: CKAP2, DPP4, CAB39, or STRA13. In some embodiments, the tumor-associated proteins or fragments thereof can comprise or a fragment having at least 80 percent sequence homology with a sequence as shown in SEQ ID NO: 2, SEQ ID NO: 3, SEQ ID NO: 4, or SEQ ID NO: 5. Also disclosed herein are methods further comprising measuring a binding of a moiety to at least one of: CKAP2, DPP4, CAB39, or STRA13, or a fragment of any of the above. Also disclosed herein are methods further comprising comparing the binding to a reference binding. In some embodiments, the reference binding can be obtained by associating a second sample from a second subject diagnosed with a cancer with one or more isolated and purified tumor-associated proteins or fragments thereof. Also disclosed herein are methods further comprising determining if the binding is higher or lower than the reference binding. In some embodiments, the moiety can comprise an autoantibody. In some embodiments, the subject may have been previously diagnosed with the cancer. In some embodiments, the cancer can comprise a lung cancer. In some embodiments, the lung cancer can be small-cell lung cancer (SCLC) or non-small cell lung cancer (NSCLC). In some embodiments, the lung cancer can be early stage lung cancer. In some embodiments, the lung cancer can be late stage lung cancer. In some embodiments, the subject and the second subject can be different. In some embodiments, the subject and the second subject can be the same. In some embodiments, the contacting can be performed before the associating. In some embodiments, the associating can be performed after the subject has been administered a treatment for the lung cancer. In some embodiments, the contacting can be performed before the subject has been administered a treatment for the lung cancer. In some embodiments, the binding can be measured by contacting the moiety with a probe. In some embodiments, the probe can be an anti-immunoglobulin antibody. In some embodiments, the probe can comprise or be associated with a chromophoric marker. In some embodiments, the chromophoric marker can be a fluorescent marker. In some embodiments, a lower binding compared to the reference binding may be indicative of a metastatic cancer. In some embodiments, the metastatic cancer can be lung cancer. In some embodiments, the metastatic lung cancer can be substantially located in a bone tissue. In some embodiments, the method can further comprise repeating at different time points the steps of: (a) contacting a sample from a subject with one or more isolated and purified tumor-associated proteins or fragments thereof, wherein the one or more tumor-associated proteins or fragments thereof comprise at least one of: CKAP2, DPP4, CAB39, or STRA13, or a fragment having at least 80 percent sequence homology with a sequence as shown in SEQ ID NO: 2, SEQ ID NO: 3, SEQ ID NO: 4, or SEQ ID NO: 5; and (b) measuring a binding of an moiety to at least one of: CKAP2, DPP4, CAB39, or STRA13, or a fragment of any of the above. In some embodiments, the different time points can be within 1 week, 2 weeks, 1 month, 2 months, 3 months, 6 months, 8 months or 1 year.

Also disclosed herein are methods comprising contacting a sample from a subject with two or more isolated and purified tumor-associated proteins or fragments thereof. In some embodiments the two or more tumor-associated proteins or fragments thereof can comprise two or more of: CKAP2, DPP4, CAB39, or STRA13, an antigenic fragment of any of these, or a polypeptide having at least 80 percent sequence homology with a sequence as shown in SEQ ID NO: 2, SEQ ID NO: 3, SEQ ID NO: 4, or SEQ ID NO: 5, or a fragment of any of the above. In some embodiments, the method can further comprise detecting a binding of a moiety to at least one of: CKAP2, DPP4, CAB39, STRA13, or a fragment of any of the above. In some embodiments, the method can further comprise comparing the binding to a reference binding. In some embodiments, the two or more isolated and purified tumor-associated proteins or fragments thereof can comprise less than 10,000 sequentially unique purified tumor-associated proteins or fragments thereof. In some embodiments percent identity can be determined by a sequence alignment performed using BLAST.

Also disclosed herein are methods comprising contacting a sample from a subject with two or more isolated and purified tumor-associated proteins or fragments thereof. In some embodiments, the two or more tumor-associated proteins or fragments thereof can comprise two or more of: CKAP2, DPP4, CAB39, or STRA13, or an antigenic fragment of any of these. In some embodiments, a tumor associated protein or fragment thereof can comprise a polypeptide having at least 80 percent sequence homology with a sequence as shown in SEQ ID NO: 2, SEQ ID NO: 3, SEQ ID NO: 4, or SEQ ID NO: 5. In some embodiments, the two or more tumor-associated proteins or fragments thereof can comprise a fragment of a polypeptide having at least 80% sequence homology with a sequence as shown in SEQ ID NO:2, SEQ ID NO:3, SEQ ID NO:4, or SEQ ID NO:5. Also disclosed herein are methods further comprising detecting a binding of an moiety to at least one of: CKAP2, DPP4, CAB39, STRA13, or a fragment of any of the above. Also disclosed herein are methods further comprising comparing the binding to a reference binding. Also disclosed herein are methods further comprising characterizing the subject as having a metastatic cancer if the binding is lower than the reference binding. In some embodiments, the moiety can be an autoantibody. In some embodiments, the reference binding can be obtained by associating a second sample from a second subject with the two or more isolated and purified tumor-associated proteins or fragments thereof. In some embodiments, the second subject can have a cancer. In some embodiments, the cancer can be lung cancer. In some embodiments, the method can further comprise determining that the binding is higher than the reference binding. In some embodiments, the metastatic cancer can be a metastatic lung cancer. In some embodiments, the metastatic lung cancer can be substantially located in a bone tissue. In some embodiments, the metastatic lung cancer can be small-cell lung cancer (SCLC) or non-small cell lung cancer (NSCLC). In some embodiments, the subject may have been previously administered a treatment for a lung cancer. In some embodiments, the contacting can be performed before the subject has been administered the treatment for the lung cancer. In some embodiments, the contacting can be performed after the subject has been administered the treatment for the lung cancer. In some embodiments, the detecting can comprise associating the moiety with a probe. In some embodiments, the probe can be an anti-immunoglobulin antibody. In some embodiments, the probe can comprise or can be associated with a fluorescent marker. In some embodiments, the subject can be human. In some embodiments, the sample can comprise a body fluid. In some embodiments, the body fluid can comprise blood or a fraction thereof. In some embodiments, the blood fraction can comprise plasma or serum. In some embodiments, the contacting can be performed on a solid support. In some embodiments, the solid support can be an array. In some embodiments, the array can comprise a substrate. In some embodiments, the substrate can comprise an epoxy resin. In some embodiments, the one or more tumor-associated proteins or fragments thereof can be covalently associated with the solid support. In some embodiments, the one or more isolated and purified tumor-associated proteins or fragments thereof can be non-covalently associated with the solid support. In some embodiments, the contacting or the detecting can be performed using a portable device. In some embodiments, the methods can further comprise repeating at different time points the steps of: (a) contacting a sample from a subject with two or more isolated and purified tumor-associated proteins or fragments thereof, wherein the two or more tumor-associated proteins or fragments thereof comprise two or more of: CKAP2, DPP4, CAB39, or STRA13, or a fragment having at least 80 percent sequence homology with a sequence as shown in SEQ ID NO: 2, SEQ ID NO: 3, SEQ ID NO: 4, or SEQ ID NO: 5; and (b) detecting a binding of an moiety to at least one of: CKAP2, DPP4, CAB39, STRA13, or a fragment of any of the above. In some embodiments, the different time points can be within 1 week, 2 weeks, 1 month, 2 months, 3 months, 6 months, 8 months or 1 year.

Also disclosed herein are solid supports comprising: two or more isolated and purified tumor-associated proteins or fragments thereof attached thereto, wherein at least two of the two or more isolated and purified tumor-associated proteins or fragments thereof can be selected from: p53, ETHE1, CTAG1A, C1QTNF1, TEX264, CLDN2, NSG1, HRAs, CKAP2, DPP4, CAB39, STRA13, or a fragment of any of the above. In some embodiments, the solid supports can further comprise a moiety bound to one of the two or more tumor-associated proteins or fragments thereof. In some embodiments, the two or more tumor-associated proteins or fragments thereof can be covalently attached to the solid support. In some embodiments, the solid support can comprise less than 10000 sequentially unique purified and isolated tumor associated proteins or fragments thereof. In some embodiments, the solid supports can further comprise an affinity ligand. In some embodiments, the affinity ligand can comprise an antigen, an antibody, an antibody fragment, glutathione, calmodulin, biotin, streptavidin, streptactin, amylose, or a metal chelate. In some embodiments, the metal chelate can comprise nickel, cobalt, zinc, mercury, or iron chelate. In some embodiments, the affinity ligand can at least partially coat a surface of the solid support. In some embodiments, at least one of the two or more isolated and purified tumor-associated proteins or fragments thereof can comprise an affinity tag. In some embodiments, the affinity tag can comprise an antigen tag, an antibody tag, an antibody fragment tag, a calmodulin tag, a glutathione S-transferase (GST) tag, a histidine (His) tag, a streptavidin tag, an avidin tag, a maltose-binding protein tag, or a Flag tag. In some embodiments, the affinity tag can be coupled to the solid support covalently. In some embodiments, the affinity tag can be coupled to the solid support non-covalently. In some embodiments, the affinity tag can be coupled to the solid support by a linker. In some embodiments, the affinity ligand can be between the solid support and at least one of the two or more tumor-associated proteins or fragments thereof. In some embodiments, at least one of the two or more tumor-associated proteins or fragments thereof can be coupled to the affinity ligand covalently. In some embodiments, at least one of the two or more tumor-associated proteins or fragments thereof can be coupled to the affinity ligand non-covalently. In some embodiments, the affinity ligand can be between the solid support and the affinity tag. In some embodiments, the affinity tag can be coupled to the affinity ligand covalently. In some embodiments, the affinity tag can be coupled to the affinity ligand non-covalently. In some embodiments are arrays comprising the solid supports described herein. In some embodiments are methods of making arrays comprising the solid supports described herein comprising coupling at least two isolated and purified tumor-associated proteins or fragments thereof to the solid support, wherein the at least two tumor-associated proteins or fragments thereof can be selected from p53, ETHE1, CTAG1A, C1QTNF1, TEX264, CLDN2, NSG1, HRAs, CKAP2, DPP4, CAB39, or STRA13, or a fragment of any of the above. In some embodiments are arrays formed the by the method above.

Also described herein are in vitro compositions comprising at least two isolated and purified tumor-associated proteins or fragments thereof, wherein the at least two isolated and purified tumor-associated proteins comprises ETHE1. In some embodiments, the compositions can further comprise a moiety bound to the ETHE1, wherein the moiety can be present in a sample obtained from a subject suspected of having a cancer. In some embodiments, the sample can comprise a body fluid. In some embodiments, the body fluid comprises blood or a fraction thereof. In some embodiments, the blood fraction can be plasma or serum. In some embodiments, the subject can be human. In some embodiments, the cancer can be lung cancer.

Also described herein are devices comprising a memory that stores executable instructions. In some embodiments, the devices can further comprise a processor that executes the executable instructions such that the device performs any of the methods described herein.

Also described herein are devices comprising a solid support, wherein the solid support can be associated with two or more isolated and purified tumor-associated proteins or fragments thereof, wherein the two or more tumor-associated proteins comprise ETHE1. In some embodiments, the devices can further comprise a processor for detecting a signal, wherein the signal can be indicative of a binding of an moiety to at least one of the two or more tumor-associated proteins or fragments thereof. In some embodiments, the signal can be at least about 2 standard deviations greater than a reference signal. In some embodiments, the reference signal can be detected after contacting a sample obtained from a non-disease subject with the two or more isolated and purified tumor-associated proteins or fragments thereof. In some embodiments, the two or more isolated and purified tumor-associated proteins or fragments thereof can further comprise p53, CTAG1A, C1QTNF1, TEX264, CLDN2, NSG1, or HRas, CKAP2, DPP4, CAB39, or STRA13, or a fragment of any of the above. In some embodiments, the solid support can be an array. In some embodiments, the two or more isolated and purified tumor-associated proteins or fragments thereof can be covalently associated with the solid support. In some embodiments, the two or more isolated and purified tumor-associated proteins or fragments thereof can be non-covalently associated with the solid support.

Also described herein are kits comprising the solid supports described herein. In some embodiments, the kits can further comprise a detecting reagent to examine the binding of a moiety to at least one of the isolated and purified tumor-associated proteins or fragments thereof. In some embodiments, the kits can further comprise instructions for use thereof.

Also described herein are computer systems comprising an electronic device, wherein said electronic device can comprise a non-transitory computer-readable medium comprising instructions that, when executed by said computer system, cause said computer system to perform: (a) contacting a sample from a subject with one or more isolated and purified tumor-associated proteins or fragments thereof, wherein the one or more isolated and purified tumor-associated proteins or fragments thereof can comprise ethylmalonic encephalopathy protein 1 (ETHE1) or a fragment having at least 80 percent sequence homology with a sequence as shown in SEQ ID NO: 1; (b) detecting a binding of at least one of the one or more isolated and purified tumor-associated proteins or fragments thereof to a moiety; and (c) characterizing the subject as having an increase probability of having lung cancer, wherein the characterizing can be performed with a sensitivity of at least about 52%.

### INCORPORATION BY REFERENCE

All publications, patents, and patent applications herein are incorporated by reference in their entireties. In the event of a conflict between a term herein and a term in an incorporated reference, the term herein controls.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 depicts a schematic design of a study identifying serological biomarkers for lung cancer.

FIG. 2A depicts serum profiling assays on HuProt arrays in Phase I. The fluorescence image represents serum profiling results from a portion of a HuProt array probed with Cy5- and Cy3-labeled anti-human IgG and -IgM antibodies, respectively. Cy5 produces a red signal and Cy3 produces a green signal.

FIG. 2B depicts an examples of a positive proteins from a serum profiling assay, in which p53 was recognized by IgG antibodies of a LC patient. Fluorescence scanning and imaging results in a red signal from the Cy5-linked anti-IgG antibody.

FIG. 2C depicts an examples of a positive protein from a serum profiling assay, in which YARS was recognized by IgM antibodies of a LC patient. Fluorescence scanning and imaging results in a green signal from the Cy3-linked anti-IgM antibody.

FIG. 2D depicts an examples of a positive protein from a serum profiling assay, in which p53 was not recognized by IgG antibodies of a healthy patient.

FIG. 2E depicts an examples of a positive protein from a serum profiling assay, in which YARS was not recognized by IgM antibodies of a healthy patient.

FIG. 3A depicts scatter plot analysis of sensitivity and specificity for all the proteins on the HuProt arrays in the anti-IgG channel in Phase I. Each dot represents a protein. Red boxed dots represent those that were selected for Phase II studies.

FIG. 3B depicts scatter plot analysis of sensitivity and specificity for all proteins on the HuProt arrays in the anti-IgM channel in Phase I. Each dot represents a protein. Red boxed dots represent those that were selected for Phase II studies.

FIG. 4A depicts the signal distribution from p53 and a negative control group in a Phase II biomarker discovery assay performed on a LC focused array. The box plot shows that the signal intensity of p53 was significantly higher in the early LC group than in the control group.

FIG. 4B depicts a Receiver Operating Characteristics (ROC) curve for p53 in a Phase II biomarker discovery assay performed on an LC focused array. The calculated Area Under the Curve (AUC) value of 0.809, sensitivity value of 24.1%, and specificity value of 93.8% for p53 are shown as obtained at the optimal cutoff value.

FIG. 4C depicts the signal distribution for p53 and a negative control group in a Phase II biomarker validation assay performed on a LC focused array. The box plot shows that the signal intensity of p53 was significantly higher in the early LC group than in the control group.

FIG. 4D depicts the signal distribution from ETHE1 and a negative control group in a Phase II biomarker discovery assay performed on a LC focused array. The box plot shows that the signal intensity of ETHE1 was significantly higher in the early LC group than in the control group.

FIG. 4E depicts a Receiver Operating Characteristics (ROC) curve for ETHE1 in a Phase II biomarker discovery assay performed on an LC focused array. The calculated Area Under the Curve (AUC) value of 0.785, sensitivity value of 32.2%, and specificity value of 91.5% for ETHE1 are shown as obtained at the optimal cutoff value.

FIG. 4F depicts the signal distribution for ETHE1 and a negative control group in a Phase II biomarker validation assay performed on a LC focused array. The box plot shows that the signal intensity of ETHE1 was significantly higher in the early LC group than in the control group.

FIG. 4G depicts the signal distribution from HRas and a negative control group in a Phase II biomarker discovery assay performed on a LC focused array. The box plot shows that the signal intensity of HRas was significantly higher in the early LC group than in the control group.

FIG. 4H depicts a Receiver Operating Characteristics (ROC) curve for HRas in a Phase II biomarker discovery assay performed on an LC focused array. The calculated Area Under the Curve (AUC) value of 0.692, sensitivity value of 13.8%, and specificity value of 96.9% for HRas are shown as obtained at the optimal cutoff value.

FIG. 4I depicts the signal distribution for HRas and a negative control group in a Phase II biomarker validation assay performed on a LC focused array. The box plot shows that the signal intensity of HRas was significantly higher in the early LC group than in the control group.

FIG. 5A depicts performance of p53, ETHE1, and HRas, in the Phase II discovery stage. Orange and light blue lines represent samples scored as positives and negatives, respectively. A sample was predicted as LC positive when any one of the three proteins in this panel scored positive (i.e. k=1). Values of sensitivity and specificity are listed.

FIG. 5B depicts performance of p53, ETHE1, and HRas, in the Phase II validation stage. Orange and light blue lines represent samples scored as positives and negatives, respectively. A sample was predicted as LC positive when any one of the three proteins in this panel scored positive (i.e. k=1). Values of sensitivity and specificity are listed.

FIG. 6A depicts performance of the p53, ETHE1, and HRas biomarker panel in late LC. Positive rates, defined as percentage of samples scored as positives in each disease category, are shown at the bottom. Orange and light blue lines represent samples scored as positives and negatives, respectively. A sample was predicted as LC positive when any one of the three proteins in this panel scored positive (i.e. k=1).

FIG. 6B depicts performance of the p53, ETHE1, and HRas biomarker panel in rectal cancer (RC), liver cancer (LiC), cervical cancer (CC), esophagus cancer (EC), and gastric cancer (GC). Positive rates, defined as percentage of samples scored as positives in each disease category, are shown at the bottom. Orange and light blue lines represent samples scored as positives and negatives, respectively. A sample was predicted as LC positive when any one of the three proteins in this panel scored positive (i.e. k=1).

FIG. 7A depicts the signal distribution, as box plots, of ELISA validation assays for p53 using samples from the Phase II study. P values are calculated between the early LC group and each of the control, healthy, and pneumonia (LBL) groups. A P value is also calculated between the healthy and pneumonia (LBL) group.

FIG. 7B depicts the signal distribution, as box plots, of ELISA validation assays for p53 using newly recruited samples. P values are calculated between the early LC group and each of the control, healthy, and pneumonia (LBL) groups. A P value is also calculated between the healthy and pneumonia (LBL) group.

FIG. 7C depicts the signal distribution, as box plots, of ELISA validation assays for ETHE1 using samples from the Phase II study. P values are calculated between the early LC group and each of the control, healthy, and pneumonia (LBL) groups. A P value is also calculated between the healthy and pneumonia (LBL) group

FIG. 7D depicts the signal distribution, as box plots, of ELISA validation assays for ETHE1 using newly recruited samples. P values are calculated between the early LC group and each of the control, healthy, and pneumonia (LBL) groups. A P value is also calculated between the healthy and pneumonia (LBL) group.

FIG. 7E depicts the signal distribution, as box plots, of ELISA validation assays for HRas using samples from the Phase II study. P values are calculated between the early LC group and each of the control, healthy, and pneumonia (LBL) groups. A P value is also calculated between the healthy and pneumonia (LBL) group

FIG. 7F depicts the signal distribution, as box plots, of ELISA validation assays for HRas using newly recruited samples. P values are calculated between the early LC group and each of the control, healthy, and pneumonia (LBL) groups. A P value is also calculated between the healthy and pneumonia (LBL) group.

FIG. 8A depicts performance of the p53, ETHE1, and HRas biomarker panel in a 115 subject group of early stage LC patients. Positive rates, defined as percentage of samples scored as positives in each disease category, are shown at the bottom. Orange and light blue lines represent samples scored as positives and negatives, respectively. A sample was predicted as LC positive when any one of the three proteins in this panel scored positive (i.e. k=1).

FIG. 8B depicts performance of the p53, ETHE1, and HRas biomarker panel in a 141 subject group of late stage LC patients. Positive rates, defined as percentage of samples scored as positives in each disease category, are shown at the bottom. Orange and light blue lines represent samples scored as positives and negatives, respectively. A sample was predicted as LC positive when any one of the three proteins in this panel scored positive (i.e. k=1).

FIG. 8C depicts performance of the p53, ETHE1, and HRas biomarker panel in a group of 97 healthy subjects. Positive rates, defined as percentage of samples scored as positives in each disease category, are shown at the bottom. Orange and light blue lines represent samples scored as positives and negatives, respectively. A sample was predicted as LC positive when any one of the three proteins in this panel scored positive (i.e. k=1).

FIG. 8D depicts performance of the p53, ETHE1, and HRas biomarker panel in a 102 subject group of late pneumonia patients. Positive rates, defined as percentage of samples scored as positives in each disease category, are shown at the bottom. Orange and light blue lines represent samples scored as positives and negatives, respectively. A sample was predicted as LC positive when any one of the three proteins in this panel scored positive (i.e. k=1).

FIG. 9A depicts the signal distribution for CKAP2 in a biomarker discovery assay for bone metastasis in stage IV NSCLC patients studied in Phase II. Box plots show that the signal intensity of CKAP2 is significantly lower in patients who developed bone metastasis than in the patients who did not develop bone metastasis.

FIG. 9B depicts the signal distribution for DPP4 in a biomarker discovery assay for bone metastasis in stage IV NSCLC patients studied in Phase II. Box plots show that the signal intensity of DPP4 is significantly lower in patients who developed bone metastasis than in the patients who did not develop bone metastasis.

FIG. 9C depicts he signal distribution for CAB39 in a biomarker discovery assay for bone metastasis in stage IV NSCLC patients studied in Phase II. Box plots show that the signal intensity of CAB39 is significantly lower in patients who developed bone metastasis than in the patients who did not develop bone metastasis.

FIG. 9D depicts a signal distribution for STRA13 in a biomarker discovery assay for bone metastasis in stage IV NSCLC patients studied in Phase II. Box plots show that the signal intensity of STRA13 is significantly lower in patients who developed bone metastasis than in the patients who did not develop bone metastasis.

FIG. 10A depicts a signal distribution for CKAP2 in a biomarker discovery assay for surgical prognosis in 13 early stage and 1 late stage LC patient. Box plots show that the signal intensity of CKAP2 is significantly lower in patients prior to surgery.

FIG. 10B depicts a signal distribution for DPP4 in a biomarker discovery assay for surgical prognosis in 13 early stage and 1 late stage LC patient. Box plots show that the signal intensity of DPP4 is significantly lower in patients prior to surgery.

FIG. 10C depicts a signal distribution for CAB39 in a biomarker discovery assay for surgical prognosis in 13 early stage and 1 late stage LC patient. Box plots show that the signal intensity of CAB39 is significantly lower in patients prior to surgery.

FIG. 10D depicts a signal distribution for STRA13 in a biomarker discovery assay for surgical prognosis in 13 early stage and 1 late stage LC patient. Box plots show that the signal intensity of STRA13 is significantly lower in patients prior to surgery.

FIG. 11A depicts a signal distribution from CTAG1A and a negative control group in a Phase II biomarker discovery assay performed on a LC focused array. The box plot shows that the signal intensity of CTAG1A was significantly higher in the early LC group than in the control group.

FIG. 11B depicts a Receiver Operating Characteristics (ROC) curve for CTAG1A in a Phase II biomarker discovery assay performed on an LC focused array. The calculated Area Under the Curve (AUC) value of 0.784, sensitivity value of 17.2%, and specificity value of 96.1% for CTAG1A are shown as obtained at the optimal cutoff value.

FIG. 11C depicts a signal distribution for CTAG1A and a negative control group in a Phase II biomarker validation assay performed on a LC focused array. The box plot shows that the signal intensity of CTAG1A was significantly higher in the early LC group than in the control group.

FIG. 12A depicts a signal distribution from TEX264 and a negative control group in a Phase II biomarker discovery assay performed on a LC focused array. The box plot shows that the signal intensity of TEX264 was significantly higher in the early LC group than in the control group.

FIG. 12B depicts a Receiver Operating Characteristics (ROC) curve for TEX264 in a Phase II biomarker discovery assay performed on an LC focused array. The calculated Area Under the Curve (AUC) value of 0.759, sensitivity value of 23.0%, and specificity value of 92.2% for TEX264 are shown as obtained at the optimal cutoff value.

FIG. 12C depicts the signal distribution for TEX264 and a negative control group in a Phase II biomarker validation assay performed on a LC focused array. The box plot shows that the signal intensity of TEX264 was significantly higher in the early LC group than in the control group.

FIG. 13A depicts the signal distribution from NSG1 and a negative control group in a Phase II biomarker discovery assay performed on a LC focused array. The box plot shows that the signal intensity of NSG1 was significantly higher in the early LC group than in the control group.

FIG. 13B depicts a Receiver Operating Characteristics (ROC) curve for NSG1 in a Phase II biomarker discovery assay performed on an LC focused array. The calculated Area Under the Curve (AUC) value of 0.740, sensitivity value of 27.6%, and specificity value of 91.5% for NSG1 are shown as obtained at the optimal cutoff value.

FIG. 13C depicts the signal distribution for NSG1 and a negative control group in a Phase II biomarker validation assay performed on a LC focused array. The box plot shows that the signal intensity of NSG1 was significantly higher in the early LC group than in the control group.

FIG. 14A depicts the signal distribution from C1QTNF1 and a negative control group in a Phase II biomarker discovery assay performed on a LC focused array. The box plot shows that the signal intensity of C1QTNF1 was significantly higher in the early LC group than in the control group.

FIG. 14B depicts a Receiver Operating Characteristics (ROC) curve for C1QTNF1 in a Phase II biomarker discovery assay performed on an LC focused array. The calculated Area Under the Curve (AUC) value of 0.763, sensitivity value of 26.4%, and specificity value of 90.7% for NSG1 are shown as obtained at the optimal cutoff value.

FIG. 14C depicts the signal distribution for C1QTNF1 and a negative control group in a Phase II biomarker validation assay performed on a LC focused array. The box plot shows that the signal intensity of C1QTNF1 was significantly higher in the early LC group than in the control group.

FIG. 15A depicts the signal distribution from CLDN2 and a negative control group in a Phase II biomarker discovery assay performed on a LC focused array. The box plot shows that the signal intensity of CLDN2 was significantly higher in the early LC group than in the control group

FIG. 15B depicts a Receiver Operating Characteristics (ROC) curve for CLDN2 in a Phase II biomarker discovery assay performed on an LC focused array. The calculated Area Under the Curve (AUC) value of 0.744, sensitivity value of 26.4%, and specificity value of 90.7% for CLDN2 are shown as obtained at the optimal cutoff value.

FIG. 15C depicts the signal distribution for CLDN2 and a negative control group in a Phase II biomarker validation assay performed on a LC focused array. The box plot shows that the signal intensity of CLDN2 was significantly higher in the early LC group than in the control group.

FIG. 16 shows an exemplary method for assessing LC in a subject.

### DETAILED DESCRIPTION

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

Some inventive embodiments herein contemplate numerical ranges. When ranges are present, the ranges include the range endpoints. Additionally, every sub range and value within the rage is present as if explicitly written out. The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. The term “about” has the meaning as commonly understood by one of ordinary skill in the art. In some embodiments, the term “about” refers to ±10%. In some embodiments, the term “about” refers to ±5%.

### Overview

Currently, screening and diagnosis of early stage LC mainly depends on high-resolution (or low-dose) chest computed tomography (CT) and biopsy. In some cases, chest CT and biopsy are not desirable tools due to the high-cost and invasiveness of the procedures. Alternatively, the detection of tumor antigens, such as CA125, CA199, neuron specific enolase (NSE), squamous cell carcinoma (SCC), carcinoembryonic antigen (CEA) and cytokeratin 19 fragment (CYFRA 21-1), in serum samples in LC patients, have been reported and suggested as diagnostic markers. However, these reports do not include specificity testing, and the sensitivity of CA125, CA199, NSE, SCC, CEA and CYFRA 21-1 has been shown to be about 5.0%, 4.9%, 19.7%, 10.9%, 17.2%, and 26.5%, respectively, in patients with stage 1 NSCLC. In addition, tumor antigens such as CYFRA 21-1 are found elevated in serum samples of patients with radiation pneumonitis, and thus has limited their use in distinguishing LC from pneumonitis. Furthermore, many tumor antigens have been found in serum samples collected from patients having non LC cancers, raising concerns about the non-specificity of these markers in LC diagnosis.

Detecting autoantibodies is a promising approach for early diagnosis of LC because humoral immune responses to autologous cellular antigens, called tumor associated antigens (TAAs), could exist several months or years prior to clinical symptoms. Tumor-associated autoantibodies often target the mutated, modified, or aberrantly expressed proteins in tumor cells, and could also act as immunologic indicators, uncovering molecular events of tumorigenesis. Several autoantibodies against TAAs, such as p53, ubiquilin 1, cyclin Y, livin, and survivin, have been found to be readily detectable in serum samples collected from LC patients. However, these reported autoantibody-based serological biomarkers do not provide sufficient sensitivity or specificity for LC diagnosis, let alone early LC diagnosis. Therefore, it is urgent and essential to identify better serological biomarkers as an economic, straightforward, and noninvasive method for the diagnosis of early stage LC with high sensitivity and specificity.

Described herein are biomarker panels identified by HuProt arrays capable of early stage LC diagnosis with >50% sensitivity and >90% specificity. HuProt arrays can comprise >20,000 individual purified full-length human proteins (i.e., HuProt v3.0), to enable a proteome-wide, unbiased screening platform for serological biomarkers. The panels described herein can detect both early stage SCLC and NSCLC with similar performance, indicating potential broad applications in LC diagnosis.

### Definitions

The terms “attach”, “bind”, “couple”, and “link” are used interchangeably and refer to covalent interactions (e.g., by chemically coupling), or non-covalent interactions (e.g., ionic interactions, hydrophobic interactions, hydrogen bonds, hybridization, etc.). The terms “specific”, “specifically”, or specificity” refer to the preferential recognition, contact, and formation of a stable complex between a first molecule and a second molecule compared to that of the first molecule with any one of a plurality of other molecules (e.g., substantially less to no recognition, contact, or formation of a stable complex between the first molecule and any one of the plurality of other molecules). For example, two molecules may be specifically attached, specifically bound, specifically coupled, or specifically linked. For example, specific hybridization between a first polynucleotide and a second polynucleotide can refer to the binding, duplexing, or hybridizing of the first polynucleotide preferentially to a particular nucleotide sequence of the second polynucleotide under stringent conditions. Sufficient number complementary base pairs in a polynucleotide sequence may be required to specifically hybridize with a target nucleic acid sequence. A high degree of complementarity may be needed for specificity and sensitivity involving hybridization, although it need not be 100%.

The term “about” or “approximately” can mean within an acceptable error range for the particular value as determined by one of ordinary skill in the art, which will depend in part on how the value is measured or determined, i.e., the limitations of the measurement system. For example, “about” can mean about plus or minus 10%, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “subject”, “patient” or “individual” as used herein can encompass a mammal and a non-mammal. A mammal can be any member of the Mammalian class, including but not limited to a human; a non-human primates such as a chimpanzee, an ape or other monkey species; a farm animal such as cattle, a horse, a sheep, a goat, a swine; a domestic animal such as a rabbit, a dog (or a canine), and a cat (or a feline); or a laboratory animal including a rodent, such as a rat, a mouse and a guinea pig, and the like. A non-mammal can include a bird, a fish and the like. In some embodiments, a subject can be a mammal. In some embodiments, a subject can be a human. In some instances, the human can be male or female. In some instances, the human can be an adult. In some instances, the human can be a child. In some instances, the human can be age 0-17 years old. In some instances, the human can be age 18-130 years old. In some instances, the subject can be diagnosed with, or suspected of having, a condition or disease such as cancer. In instances, the subject can be diagnosed with, or suspected of having lung cancer.

The term “non-diseased subject” as used herein can encompass a healthy individual. A healthy individual can be an individual without a particular disease. For example, a healthy individual can be an individual without a cancer such as lung cancer.

The terms “treat,” “treating”, “treatment,” “ameliorate” or “ameliorating” and other grammatical equivalents as used herein, can include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms can further include achieving a therapeutic benefit and/or a prophylactic benefit. Therapeutic benefit can mean eradication or amelioration of the underlying disease being treated. Also, a therapeutic benefit can be achieved with the eradication or amelioration of one or more of the physiological symptoms associated with the underlying disease such that an improvement can be observed in the patient, notwithstanding that, in some embodiments, the patient can still be afflicted with the underlying disease.

The term “isolated” as used herein refers to the removal of a protein, such as a tumor-associated protein, from or from at least part of its endogenous or natural environment. For example, a protein may isolated by removal and separation from an organism, bodily fluid, or fermentation medium.

The term “purified” as used herein defines the purity of a protein, such as a tumor-associated protein, and refers to proteins that are substantially free of other human or non-human proteins of natural or endogenous origin. A purified protein may contain less than about 20%, 15%, 10%, 5%, 4%, 3%, 2%, or 1% by mass of protein contaminants residual of the purification process. Purified proteins, however, may contain other proteins added as stabilizers, carriers, excipients, binding agents, or purification tags. In some aspects, purified proteins may be detectable as a substantially single band on a polyacrylamide gel using a silver or Coomasie stain.

The term “probe” as used herein refers to any molecular moiety that can be identified to detect the presence of the probe. A probe may be used to detect a target molecule or moiety to which the probe is associated. The association of a probe to a target may be covalent or non-covalent. In some embodiments, a probe may comprise any detectable moiety, such as a fluorescent dye, a phosphor, a radiolabel, or a chromophore. The term “fluorescent dye” as used herein refers to molecular moiety comprising a fluorophore that can be used as a probe. In some aspects, a chromophore may absorb, reflect, or emit light at ultraviolet or visible wavelengths.

The terms “early stage” and “late stage” as used herein refer to the degree of progression of a cancer. For example, an early stage lung cancer can be a stage I lung cancer or a stage II lung cancer. In some instances, an early stage lung cancer can be confined to the lung tissue of a subject. In some instances, an early stage lung cancer can be a limited stage small cell lung cancer. In some instances, a late stage lung cancer can be a stage III or stage IV lung cancer. In some instances, a late stage lung cancer can have spread from the lung tissue to another organ of a subject. In some instances, a late stage lung cancer can be an extensive stage small cell lung cancer.

The terms “homologous,” “homology,” or “percent homology” as used herein refer to the degree of sequence similarity between an amino acid or nucleotide sequence and a reference sequence. In some instances, percent sequence homology can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application. As used herein, the term “homology” can be used interchangeably with the term “identity.”

As used herein, the term “cell-free” refers to the condition of the nucleic acid sequence as it appeared in the body before the sample is obtained from the body. For example, circulating cell-free nucleic acid sequences in a sample may have originated as cell-free nucleic acid sequences circulating in the bloodstream of the human body. In contrast, nucleic acid sequences that are extracted from a solid tissue, such as a biopsy, are generally not considered to be “cell-free.” In some cases, cell-free DNA may comprise fetal DNA, maternal DNA, or a combination thereof. In some cases, cell-free DNA may comprise DNA fragments released into a blood plasma. In some cases, the cell-free DNA may comprise circulating tumor DNA. In some cases, cell-free DNA may comprise circulating DNA indicative of a tissue origin, a disease or a condition. A cell-free nucleic acid sequence may be isolated from a blood sample. A cell-free nucleic acid sequence may be isolated from a plasma sample.

Samples

In some embodiments, a sample can be an antibody containing sample. In some embodiments, an antibody containing sample can be a biological fluid. In some embodiments, a sample can be a biological fluid. A biological fluids prepared for analysis in a process described herein include or can include a host of potential biomarkers including markers expressed on cells (non-adherent cells, including T-cells or other immune effector cells), microorganisms, proteins, peptides, lipids, polysaccharides, small molecules, organic molecules, inorganic molecules, biological molecules and including any detectable or reactable moiety in such complex milieu. In some embodiments, antibodies can be generated as a result of a disease or condition. In some embodiments, body fluids such as serum, plasma, saliva or other fluids or samples derived from a subject or animal or organism can be the source of such biomarkers. In some embodiments, a sample can be blood, serum, saliva or CSF. In some embodiments, a sample can be for example, sputum, bone marrow, synovial fluid, aqueous humor, amniotic fluid, cerumen, breast milk, broncheoalveolar lavage fluid, semen, prostatic fluid, Cowper's fluid, pre-ejaculatory fluid, female ejaculate, sweat, tears, cyst fluid, pleural fluid, peritoneal fluid, pericardial fluid, lymph, chyme, chyle, bile, interstitial fluid, menses, pus, sebum, vaginal secretion, mucosal secretion, stool water, pancreatic juice, lavage fluid from sinus cavities, bronchopulmonary aspirate, blastocyl cavity fluid, or umbilical cord blood. In some embodiments, a sample can be from a subject afflicted with a disease or condition disclosed herein. In some embodiments, a subject from which a sample is obtained can have a disease. In some embodiments, a disease can be a cancer. In some embodiments, the cancer can be lung cancer. In some embodiments, lung cancer can be SCLC. In some embodiments, SCLC can be limited stage SCLC or extensive stage SCLC. In some embodiments, lung cancer can be NSCLC. In some embodiments, NSCLC can be occult stage, stage 0, stage I, stage II, stage III, and stage IV.

Biomarkers

A biomarker can refer to a biomolecule. In some cases, a biomarker can be a biomolecule associated with a disease. When associated with a disease, a biomarker can have a profile different under the disease condition compared to a non-disease condition. A biomarker can be any class of biomolecules, including polynucleotides, polypeptides, carbohydrates and lipids. In some cases, a biomarker can be a polynucleotide. In some cases, a biomarker can be a polypeptide. In some cases, a biomarker can be an antibody. A polynucleotide can be any type of nucleic acid molecule, including DNA, RNA, a hybridization thereof, or any combination thereof. For example, a polynucleotide can be cDNA, genomic DNA, mRNA, tRNA, rRNA, or microRNA. In some cases, a polynucleotide can be a cell-free nucleic acid molecule circulating in blood or a cellular nucleic acid molecule in a cell circulating in blood. A polypeptide or protein can be contemplated to include any fragments thereof, in particular, immunologically detectable fragments. A biomarker can also include one or more fragments of the biomarker having sufficient sequence such that it still possesses the same or substantially the same function as the full-size biomarker. An active fragment of a biomarker retains 100% of the activity of the full-size biomarker, or at least about 99%, 95%, 90%, 85%, 80% 75%, 70%, 65%, 60%, 55%, or at least 50% of its activity. In certain cases, an active fragment of a biomarker can be detectable (e.g., a polypeptide detectable by an antibody, or a polynucleotide detectable by an oligonucleotide). A biomarker of a disease disclosed herein (e.g. cancer) can be a biomolecule associated with a disease. In some cases, a biomarker of a disease can be a biomolecule associated with the disease, but not associated with other diseases. In some cases, a biomarker of a disease can be a biomolecule associated with that disease and other diseases. In some embodiments, a biomarker can be an autoantibody. In some embodiments, a biomarker can be a tumor-associated protein or fragment thereof. In some embodiments, a biomarker can be tumor protein p53 (p53), ethylmalonic encephalopathy protein 1 (ETHE1), Cancer/Testis Antigen 1A (CTAG1A), C1q And Tumor Necrosis Factor Related Protein 1 (C1QTNF1), Testis Expressed 264 (TEX264), Claudin 2 (CLDN2), Neuron Specific Gene Family Member 1 (NSG1), GTPase HRas (HRas), Cytoskeleton Associated Protein 2 (CKAP2), Dipeptidyl peptidase 4 (DPP4), Calcium-binding protein 39 (CAB39), Centromere protein X (STRA13), LIM Zinc Finger Domain Containing 1 (LIMS1), Recombination Signal Binding Protein for Immunoglobulin Kappa J Region 1 (RBPJ1), Recombination Signal Binding Protein for Immunoglobulin Kappa J Region 2 (RBPJ2), Recombination Signal Binding Protein for Immunoglobulin Kappa J Region 3 (RBPJ3), Protein MAK16 Homolog (MAK16), Calcium-Binding and Coiled-Coil Domain-Containing Protein 2 (CALCOCO2), Syndecan Binding Protein (SDCBP), Galectin-8 (LGALS8), Doublesex and Mab-3 Related Transcription Factor 2 (DMRT2), or a fragment of any of the above. In some embodiments, a biomarker can be a protein selected from Table 2.

Tumor-Associated Protein

A tumor associated protein can be an antigenic substance associated with, produced in, on, or by a tumor cell. A tumor-associated protein may be known as a tumor-associated antigen. A tumor associated protein can trigger an immune response in a subject. A tumor-associated antigen can be a protein that is not exposed to the immune system in a healthy subject. Tumor-associated proteins may be sequestered from the immune system in a healthy subject. Tumor-associated proteins may be expressed in small quantities in a healthy subject. Tumor associated proteins may be expressed in greater quantities in a cancerous cell than in a non-cancerous cell. Tumor associated protein may be expressed in greater quantities in a disease subject than in a non-disease subject. Tumor associated proteins may be expressed in certain stages of development. A tumor-associated protein may be a oncofetal antigen. Tumor-associated proteins may be structurally modified from the sequence found in a healthy subject due to mutation. A tumor-associated protein may be the mutated form of a naturally expressed protein. A tumor-associated protein may be the un-mutated form of a naturally expressed protein. In some instances, a tumor associated protein may comprise at least 1, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, 200, 250, 300, 350, 400, 450, 500, 600, 700, 800, 900, 1000, 2000, 3000, 4000, 5000, 6000, 7000, 8000, 9000, 10000, 15000, 20000, 25000, 30000, 35000, or 40000 amino acids.

A tumor-associated protein may be present exclusively in, on or produced by a tumor cell. A tumor-associated antigen may be present in tumor cells and in healthy cells. In some instances, a tumor-associated protein may be expressed from a protooncogene. In some instances, a tumor-associated protein may be expressed from a tumor-suppressor gene. A tumor-associated protein may be expressed in a cell infected with an oncovirus. In some instances, a tumor-associated protein may be recognized by a subject's immune system. A tumor-associated protein may be recognized by an antibody, such as an autoantibody.

A tumor-associated protein can be, but is not limited to, a polypeptide, a protein, a protein fragment, a tagged protein, a fusion protein, an antibody, or an antibody fragment. In some instances, a tumor-associated protein can comprise at least two amide bonds. In some instances, a tumor-associated protein does not comprise a phosphodiester linkage. In some instances, a tumor-associated protein is not DNA or RNA. In some aspects one or more tumor-associated proteins can be spotted onto an array. In some instances, a tumor-associated protein may be glycosylated.

In some instances, a tumor-associated protein can comprise a polypeptide, protein, or fragment thereof. “Polypeptide” and “protein” are used interchangeably and refer to a polymer of two or more amino acids joined by a covalent bond (e.g., an amide bond). Polypeptides as described herein can include full length proteins (e.g., fully processed proteins) as well as shorter amino acid sequences (e.g., fragments of naturally-occurring proteins or synthetic polypeptide fragments). Polypeptides can include naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V) and non-naturally occurring amino acids (e.g., amino acids which is not one of the twenty amino acids commonly found in peptides synthesized in nature, including synthetic amino acids, amino acid analogs, and amino acid mimetics). A tumor-associated protein can be a biomarker.

For example, a tumor-associated protein can comprise an isolated polypeptide, a purified polypeptide, or a polypeptide within a virus particle. For example, a tumor-associated protein can comprise a polypeptide within a virus particle membrane. A virus particle refers to a fully or partially assembled capsid of a virus surrounded by a lipid envelope. A viral particle may or may not contain nucleic acids.

For example, a tumor-associated protein can comprise an antibody or fragment thereof. For example, a tumor-associated protein can comprise a transcription factor. For example, a tumor-associated protein can comprise a receptor. For example, a tumor-associated protein can comprise a transmembrane receptor.

Tumor-associated proteins can include isolated, purified, and/or recombinant polypeptides. Tumor-associated proteins include tumor-associated proteins present in a mixture of analytes (e.g., a lysate). For example, tumor-associated proteins include tumor-associated proteins present in a lysate from a plurality of cells or from a lysate of a single cell.

A tumor-associated protein can comprise a member of a specific binding pair (e.g., a ligand). A tumor-associated protein can be monovalent (monoepitopic) or polyvalent (polyepitopic). A tumor-associated protein can be antigenic or haptenic. A tumor-associated protein can be a single molecule or a plurality of molecules that share at least one common epitope or determinant site. A tumor-associated protein can be a part of a cell (e.g., a bacteria cell, a plant cell, or an animal cell). A target cell can be either in a natural environment (e.g., tissue), a cultured cell, or a microorganism (e.g., a bacterium, fungus, protozoan, or virus), or a lysed cell. A tumor-associated protein can be further modified (e.g. chemically), to provide one or more additional binding sites such as, but not limited to, a dye (e.g., a fluorescent dye), a polypeptide modifying moiety such as a phosphate group, a carbohydrate group, and the like, or a polynucleotide modifying moiety such as a methyl group.

A tumor-associated protein can comprise at least one potential binding site for an antibody. In some instances, a tumor-associated protein comprises one binding site. In some instances, a tumor-associated protein comprises at least two binding sites. For example, a tumor-associated protein can comprise 2, 3, 4, 5, 6, 7, 8, 9, 10, 11, 12, 13, 14, 15, 16, 17, 18, 19, 20, or more binding sites.

In some instances, a tumor-associated protein can be a molecule found in a sample from a host. A sample from a host includes a body fluid (e.g., urine, blood, plasma, serum, saliva, semen, stool, sputum, cerebral spinal fluid, tears, mucus, and the like). A sample can be examined directly or may be pretreated to render the tumor-associated protein more readily detectible. Samples include a quantity of a substance from a living thing or formerly living things. A sample can be natural, recombinant, synthetic, or not naturally occurring. A tumor-associated protein can be expressed by a cell naturally or recombinantly, in a cell lysate or cell culture medium, an in vitro translated sample, or an immunoprecipitation from a sample (e.g., a cell lysate).

In some instances, a tumor-associated protein is expressed in a cell-free system or in vitro. For example, a tumor-associated protein can be in a cell extract containing a nucleotide template and raw materials for translation of the tumor-associated protein. In some instances, a tumor-associated protein can be in a cell extract containing a DNA template, and reagents for transcription and translation. Exemplary sources of cell extracts that can be used include wheat germ, Escherichia coli, rabbit reticulocyte, hyperthermophiles, hybridomas, Xenopus oocytes, insect cells, and mammalian cells (e.g., human cells). Exemplary cell-free methods that can be used to express tumor-associated proteins (e.g., to produce tumor-associated protein on an array) include Protein in situ arrays (PISA), Multiple spotting technique (MIST), Self-assembled mRNA translation, Nucleic acid programmable protein array (NAPPA), nanowell NAPPA, DNA array to protein array (DAPA), membrane-free DAPA, nanowell copying and μIP-microintaglio printing, and pMAC-protein microarray copying (See Kilb et al., Eng. Life Sci. 2014, 14, 352-364).

In some instances, a tumor-associated protein can be synthesized in situ (e.g., on a solid substrate of an array) from a DNA template. In some instances, a plurality of tumor-associated proteins can be synthesized in situ from a plurality of corresponding DNA templates in parallel or in a single reaction. Exemplary methods for in situ tumor-associated protein expression include those described in Stevens, Structure 8(9): R177-R185 (2000); Katzen et al., Trends Biotechnol. 23(3):150-6. (2005); He et al., Curr. Opin. Biotechnol. 19(1):4-9. (2008); Ramachandran et al., Science 305(5680):86-90. (2004); He et al., Nucleic Acids Res. 29(15):E73-3 (2001); Angenendt et al., Mol. Cell Proteomics 5(9): 1658-66 (2006); Tao et al, Nat Biotechnol 24(10):1253-4 (2006); Angenendt et al., Anal. Chem. 76(7):1844-9 (2004); Kinpara et al., J. Biochem. 136(2):149-54 (2004); Takulapalli et al., J. Proteome Res. 11(8):4382-91 (2012); He et al., Nat. Methods 5(2):175-7 (2008); Chatterjee and J. LaBaer, Curr Opin Biotech 17(4):334-336 (2006); He and Wang, Biomol Eng 24(4):375-80 (2007); and He and Taussig, J. Immunol. Methods 274(1-2):265-70 (2003).

In some instances, tumor-associated protein synthesis can be carried out on a solid surface (e.g., an array surface) coated with a protein-capturing reagent or antibody. In some instances, a tumor-associated protein can comprise a tag (e.g., polyhistidine or GST) that is bound by the capture reagent or antibody, thus coupling the tumor-associated protein to the solid surface (e.g., a nucleic acid programmable protein array (NAPPA)). In some instances, a DNA template can be immobilized onto the same protein-capture surface. For example, the DNA template can be biotinylated and bound to avidin pre-coated onto the protein capture surface. In some instances, the DNA template may not be coupled to the solid support. In some instances, a DNA template can added as a free molecule in the reaction synthesis mixture (e.g., a protein in situ array (PISA)).

In some instances, in situ puromycin-capture methods can be used to express tumor-associated protein. For example, the template DNA can be transcribed to mRNA, and a single-stranded DNA oligonucleotide modified with biotin and puromycin on each end can be hybridized to the 3′-end of the mRNA. The mRNAs can be coupled to the surface e.g., by the binding of biotin to streptavidin that can be pre-coated on the surface. Cell extract can then be added to initiate in situ translation. When the ribosome reaches the hybridized oligonucleotide, it stalls and incorporates the puromycin molecule to the nascent polypeptide chain, thereby attaching the newly synthesized protein to the surface via the DNA oligonucleotide. Purified tumor-associated proteins may be obtained after the mRNA is removed (e.g., digested with RNase).

In some instances, DNA array to protein array (DAPA) methods can be used to repeatedly produce protein arrays by printing them from a single DNA template array, on demand. An array of immobilized DNA templates on a substrate is assembled face-to-face with a second substrate pre-coated with a protein-capturing reagent, and a membrane soaked with a cell extract is placed between the two substrates for transcription and translation. The synthesized tumor-associated proteins can then be immobilized onto a substrate to form the array.

An array can comprise a plurality of tumor-associated proteins. An array can comprise a plurality of tumor-associated proteins representing a substantial portion or an entire organism's proteome, such as a bacterial, viral, fungal, plant, or animal proteome. An array can comprise a plurality of tumor-associated proteins representing a substantial portion or an entire proteome of an insect or mammal, such as a mouse, rat, rabbit, cat, dog, monkey, goat, or human. For example, an array can comprise a plurality of tumor-associated proteins representing at least 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of an organism's proteome.

An array can comprise at least one tumor-associated protein. A array can comprise a plurality of tumor-associated proteins comprising at least 2 different tumor-associated proteins. For example, a tumor-associated protein can comprise a plurality of tumor-associated protein comprising at least 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, or 25,000 different tumor-associated proteins. For example, an array can comprise a plurality of tumor-associated proteins comprising less than 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, or 25,000 different tumor-associated proteins. In some embodiments, the plurality of tumor associated proteins can be sequentially unique purified tumor-associated proteins or fragments thereof.

In some instances, tumor-associated proteins can comprise a tag. In some embodiments, a tag is an affinity tag. Examples of such affinity tags include, but are not limited to, Glutathione-S-transferase (GST), Maltose binding protein (MBP), Green Fluorescent Protein (GFP), AviTag (a peptide allowing biotinylation by the enzyme BirA and so the protein can be isolated by streptavidin), Calmodulin-tag (a peptide bound by the protein calmodulin), polyglutamate tag (a peptide binding efficiently to anion-exchange resin such as Mono-Q), FLAG-tag (a peptide recognized by an antibody), HA-tag (a peptide recognized by an antibody), His tag (generally 5-10 histidines (SEQ ID NO: 121) which are bound by a nickel or cobalt chelate), Myc-tag (a short peptide recognized by an antibody, S-tag, SBP-tag (a peptide which binds to streptavidin), Softag 1, Strep-tag (a peptide which binds to streptavidin or the modified streptavidin called streptactin), TC tag (a tetracysteine tag that is recognized by FlAsH and ReAsH biarsenical compounds), V5 tag, Xpress tag, Isopeptag (a peptide which binds covalently to pilin-C protein), SpyTag (a peptide which binds covalently to SpyCatcher protein) or a combination thereof. In some instances, for example, tumor-associated protein can comprise a fusion tag. For example, a tumor-associated protein can comprise a GST-tag, His-tag, FLAG-tag, T7 tag, S tag, PKA tag, HA tag, c-Myc tag, Trx tag, Hsv tag, CBD tag, Dsb tag, pelB/ompT, KSI, MBP tag, VSV-G tag, 3-Gal tag, GFP tag, or a combination thereof, or other similar tags. In some instances, a protein tag binder can be a group which binds an endogenous protein tag (e.g., an epitope on the protein). In this group of embodiments, a protein tag binder can typically be an antibody or antibody fragment which is sufficient to form a non-covalent association complex with the protein tag or epitope. In some embodiments, the tumor-associated proteins comprise PTMs including, but not limited to, glycosylation, phosphorylation, acetylation, methylation, myristoylation, prenylation, or proteolytic processing. In some embodiments, a tumor-associated protein is homologous to a native polypeptide.

In some instances, a tumor-associated protein can comprises a contiguous span of at least 6 amino acids, for example, least 8, 9, 10, 12, 15, 20, 25, 30, 40, 50, or 100 amino acids of a reference sequence. In some instances, a tumor-associated protein can comprise a contiguous stretch of amino acids comprising a site of a mutation or functional mutation, including a deletion, addition, swap, or truncation of the amino acids in a polypeptide sequence. Polypeptides may be isolated from human or mammalian tissue samples or expressed from human or mammalian genes. Polypeptides may be made using routine expression methods known in the art. A polynucleotide encoding a desired polypeptide may be inserted into an expression vector suitable for any convenient host. Both eukaryotic and prokaryotic host systems can be used in forming recombinant polypeptides. A polypeptide may be isolated from lysed cells or from the culture medium and purified to the extent needed for its intended use. (See, e.g., WO2012103260 and WO2011159959). Purification may be by any technique known in the art, for example, differential extraction, salt fractionation, chromatography, centrifugation, and the like (See, e.g., Abbondanzo et al., (1993) Methods in Enzymology, Academic Press, New York. pp. 803-23).

In addition, shorter protein fragments may be produced by chemical synthesis. Alternatively proteins of the presently disclosed subject matter are extracted from cells or tissues of humans or non-human animals. Methods for purifying proteins are known in the art, and include the use of detergents or chaotropic agents to disrupt particles followed by differential extraction and separation of the polypeptides by ion exchange chromatography, affinity chromatography, sedimentation according to density, and gel electrophoresis, for example. Reference cDNA may be used to express polypeptides. A nucleic acid encoding a polypeptide to be expressed can be operably linked to a promoter in an expression vector using conventional cloning technology. For example, a polypeptide in an expression vector may comprise the full coding sequence for the polypeptide or a portion thereof.

In some embodiments, a tumor-associated protein is a membrane bound protein. In one embodiment, the membrane bound protein is CD4, a classical type I membrane protein with a single transmembrane (TM) domain. (Carr et al., (1989) J. Biol. Chem. 264:21286-95). In another embodiment, the membrane bound protein is GPR77, a multi-spanning, G-protein coupled receptor (GPCR) membrane protein. (Cain & Monk, (2002) J. Biol. Chem. 277:7165-69).

Additional exemplary membrane bound proteins include, but are not limited to, GPCRs (e.g. adrenergic receptors, angiotensin receptors, cholecystokinin receptors, muscarinic acetylcholine receptors, neurotensin receptors, galanin receptors, dopamine receptors, opioid receptors, erotonin receptors, somatostatin receptors, etc.), ion channels (e.g., nicotinic acetylcholine receptors, sodium channels, potassium channels, etc.), receptor tyrosine kinases, receptor serine/threonine kinases, receptor guanylate cyclases, growth factor and hormone receptors (e.g., epidermal growth factor (EGF) receptor), and others. Mutant or modified variants of membrane-bound proteins may also be used. For example, some single or multiple point mutations of GPCRs retain function and are involved in disease (See, e.g., Stadel et al., (1997) Trends in Pharmacological Review 18:430-37). Also provided herein are libraries of tumor-associated proteins comprising a plurality of tumor-associated proteins in which at least 5%, 10%, 20%, 30%, 40%, 50%, 60%, 70%, 80%, 90%, 99% or 100% of the tumor-associated proteins are part of a common pathway. The present invention also provides a library of tumor-associated proteins in which the library represents at least 5%, 10%, 20%, 30%, 40%, 50%, 60%, 70%, 80%, 90%, 99% or 100% of all the tumor-associated proteins that are part of a common pathway in the genome.

In some embodiments, a tumor-associated protein can be tumor protein p53 (p53), ethylmalonic encephalopathy protein 1 (ETHE1), Cancer/Testis Antigen 1A (CTAG1A), C1q And Tumor Necrosis Factor Related Protein 1 (C1QTNF1), Testis Expressed 264 (TEX264), Claudin 2 (CLDN2), Neuron Specific Gene Family Member 1 (NSG1), GTPase HRas (HRas), Cytoskeleton Associated Protein 2 (CKAP2), Dipeptidyl peptidase 4 (DPP4), Calcium-binding protein 39 (CAB39), Centromere protein X (STRA13), LIM Zinc Finger Domain Containing 1 (LIMS1), Recombination Signal Binding Protein for Immunoglobulin Kappa J Region 1 (RBPJ1), Recombination Signal Binding Protein for Immunoglobulin Kappa J Region 2 (RBPJ2), Recombination Signal Binding Protein for Immunoglobulin Kappa J Region 3 (RBPJ3), Protein MAK16 Homolog (MAK16), Calcium-Binding and Coiled-Coil Domain-Containing Protein 2 (CALCOCO2), Syndecan Binding Protein (SDCBP), Galectin-8 (LGALS8), Doublesex and Mab-3 Related Transcription Factor 2 (DMRT2), or a fragment of any of the above.

In some embodiments, a tumor-associated protein can be a protein having 70%, 75%, 80%, 85%, 90%, 95%, 96%, 97%, 98%, 99%, or 99.5% sequence homology to SEQ ID NO:1 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:2 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:3 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:4 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:5 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:6 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:7 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:8 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:9 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:10 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:11 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:12 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:13 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:14 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:15 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:16 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:17 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:18 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:19 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:20 or a fragment thereof. In some embodiments, a tumor-associated protein can be a protein having 70%, 75%, 80%, 85%, 90%, 95%, 96%, 97%, 98%, 99%, or 99.5% sequence homology with a sequence as shown in SEQ ID NO:21 or a fragment thereof.

In some embodiments, a tumor-associated protein can be a protein having 70%, 75%, 80%, 85%, 90%, 95%, 96%, 97%, 98%, 99%, or 99.5% sequence homology to a protein listed in Table 2. In some embodiments, a tumor-associated protein can be a protein having 70%, 75%, 80%, 85%, 90%, 95%, 96%, 97%, 98%, 99%, or 99.5% sequence homology to any protein disclosed herein.

Coupling of Tumor-Associated Proteins to a Solid Support

A tumor-associated protein can be coupled to a solid support (e.g., an array or bead). In some instances, a tumor-associated protein is non-covalently coupled to a solid support. For example, a non-covalent interaction can be an ionic interaction or a van der Waals interaction. In some instances, a tumor-associated protein can be covalently coupled to a solid support. In some instances, a tumor-associated protein can be reversibly coupled to a solid support. In some instances, a tumor-associated protein can be irreversibly coupled to a solid support.

A surface of a solid support can be coated with a functional group and a tumor-associated protein can be attached to the solid support through the functional group. For example, a solid support can be coated with a first functional group and a tumor-associated protein comprising a second functional group can be attached to the solid support by reacting the first functional group with the second functional group. For example, a surface of a solid support can be coated with streptavidin and a biotinylated tumor-associated protein can be attached thereto. Exemplary couplings of a tumor-associated protein include streptavidin- or avidin- to biotin interactions; hydrophobic interactions; magnetic interactions; polar interactions, (e.g., associations between two polar surfaces); formation of a covalent bond (e.g., an amide bond, disulfide bond, thioether bond, or via crosslinking agents; and via an acid-labile linker.

In some embodiments, the surface of a solid support can be coated with an affinity ligand. In some embodiments, an affinity ligand can include, but is not limited to an antigen, an antibody, an antibody fragment, glutathione, calmodulin, biotin, streptavidin, streptactin, amylose, an anion-exchange resin such as Mono-Q, FlAsH and ReAsH biarsenical compounds, pilin-C protein, SpyCatcher protein or a metal chelate. In some instances, a metal chelate can include but is not limited to nickel, cobalt, zinc, mercury, cupper or iron chelate. In some embodiments, the solid support can be coated entirely. In some embodiments, a solid support can be coated partially. In some embodiments, tumor-associated proteins can comprise an affinity tag and solid support can comprise an affinity ligand, thus coupling the tumor-associated proteins to the solid support by reacting the affinity tag with the affinity ligand.

In some instances, a tumor-associated protein can be coupled to a solid surface through a linker. For example, a first functional group of a linker attached to a solid surface can be coupled to a tumor-associated protein, thereby coupling the tumor-associated protein to the solid surface. For example, a first functional group of a linker can be coupled to a tumor-associated protein and a second functional group of the linker can be coupled to a solid support, thereby coupling the tumor-associated protein to the solid surface. Tumor-associated proteins can be coupled to a solid surface through a linker. In some instances, a linker comprising a first and a second functional group can be attached to the solid support via the second functional group after the first functional group is coupled to the tumor-associated protein. In some instances, a linker comprising a first and a second functional group can be attached to the solid support via the second functional group before the first functional group is coupled to the tumor-associated protein.

In some instances, a tumor-associated protein can be coupled to a solid surface via an antibody. For example, an antibody linker can be attached to a solid surface and a tumor-associated protein to which the antibody specifically binds can be linked to the solid support by binding to the antibody linker. In some instances, the coupling is photocleavable. In some instances, tumor-associated proteins can comprise a tag that is directly coupled to a solid surface. For example, a tumor-associated protein can comprise a fusion tag that is directly conjugated to the solid surface. For example, a tumor-associated protein can comprise a GST-tag, His-tag, FLAG-tag, or other similar tags and the tag can be directly coupled to the solid surface instead of the tumor-associated protein itself.

There are many known methods for covalently immobilizing polypeptides and antibodies onto a solid support. For example, MacBeath et al., (1999) J Am Chem Soc 121: 7967-68) use the Michael addition to link thiol-containing compounds to maleimide-derivatized glass slides to form a microarray of small molecules. (See also, Lam & Renil (2002) Current Opin Chemical Biol 6:353-58). Non-covalent coupling may be by any suitable secondary interaction, including but not limited to hydrophobic bonding, hydrogen bonding, Van der Waals interactions, ionic bonding, etc.

Amine chemistry can be used to couple or immobilize tumor-associated proteins to a solid surface. For example, a covalent amide bond can be formed between a tumor-associated protein and a solid support. For example, a covalent amide bond can be formed by reacting a carboxyl-functionalized tumor-associated protein with an amino-functionalized solid support. For example, a covalent amide bond can be formed by reacting an amide-functionalized tumor-associated protein with a carboxyl-functionalized solid support. Amine-terminated tumor-associated protein may be immobilized using amine/cyanuric chloride coupling; amide bonding through reactions with N-hydroxysuccinimide (NHS)-ester-, carboxylic acid-, carbonate-, anhydride- or acyl group-functionalized surfaces; amidine formation through reaction with imidoester-functionalized surfaces; sulphonamide formation through reactions with sulfonyl halide-functionalized surfaces; aniline formation through reactions with surface presenting aryl groups; imine formation through reactions with aldehyde-functionalized surfaces; amino ketone formation through Mannich reactions with aldehyde-functionalized surfaces; guanidine formation through reactions with carbodiimide-functionalized surfaces; urea formation through reactions with isocyanate-functionalized surfaces; thiourea formation through reactions with isothiocyanate-functionalized surfaces, or; amino alcohol formation through reactions with epoxide-functionalized surfaces. Hydrazine- or oxyamine-terminated binding agents may be immobilized in the same way.

Thiol groups can be used to couple or immobilize tumor-associated protein to a solid surface. For example, tumor-associated protein having or functionalized with thiol groups with may be immobilized on surfaces presenting, e.g., maleimide, aryl- or carbon-carbon double-bond-containing groups through formation of stable carbon-sulfur bonds, or through interactions with aziridine-functionalized surfaces. Disulfide exchange reactions with thiol-functionalized surfaces may also be used. Tumor-associated proteins having or functionalized with thiol groups may be immobilized on gold surfaces through semi-covalent interactions between gold and sulphur groups.

Carboxylic acid-functionalized surfaces may also be used to immobilize tumor-associated proteinfunctionalized with carbodiimide and diazoalkane groups. Solid surfaces presenting hydroxyl groups may be used to immobilize isocyanate- and epoxide-functionalized tumor-associated proteins.

Functionalized tumor-associated protein may also be immobilized through cycloaddition reactions between functional groups having a conjugated diene and groups having a substituted alkene through Diels-Alder chemistry, or using “click” chemistry, through reactions between nitrile and azine groups. In any of the above described covalent couplings, the tumor-associated protein-surface orientation of functional groups may be reversed. An alternative means of covalent attachment not utilizing a derivatized binding agent utilizes array surfaces having photoreactive groups such as benzophenone, diazo, diazirine, phthalamido and arylazide groups.

Non-covalent immobilization may involve electrostatic interactions between tumor-associated proteins and surfaces modified to contain positively- or negatively-charged groups, such as amine or carboxyl groups, respectively. tumor-associated proteins may be non-covalently immobilized in a defined orientation, for example, using fluorophilic, biotin-streptavidin, histidine-Ni, histidine-Co, and complementary single-stranded DNA interactions between tagged tumor-associated proteins and binding partner-coated surfaces, in either orientation.

Appropriate agents for coupling of tumor-associated proteins to a solid surface include a variety of agents that are capable of reacting with a functional group present on a surface of the tumor-associated protein and with a functional group present on the solid surface. Reagents capable of such reactivity include homo- and hetero-bifunctional reagents, many of which are known in the art. Exemplary bifunctional cross-linking agents include is N-succinimidyl(4-iodoacetyl) aminobenzoate (SIAB), dimaleimide, dithio-bis-nitrobenzoic acid (DTNB), N-succinimidyl-S-acetyl-thioacetate (SATA), N-succinimidyl-3-(2-pyridyldithio) propionate (SPDP), succinimidyl 4-(N-maleimidomethyl) cyclohexane-1-carboxylate (SMCC) and 6-hydrazinonicotimide (HYNIC). Any suitable nucleophile reactive group can be used including —NR1-NH2 (hydrazide), —NR1(C═O)NR2NH2 (semicarbazide), —NR1(C═S)NR2NH2 (thiosemicarbazide), (C═O)NR1NH2 (carbonylhydrazide), —(C═S)NR1NH2 (thiocarbonylhydrazide), —(SO2)NR1NH2 (sulfonylhydrazide), —NR1NR2(C)NR3NH2 (carbazide), —NR1NR2(C═S)NR3NH2 (thiocarbazide), and —O—NH2 (hydroxylamine), where each R1, R2, and R3 is independently H, or alkyl having 1-6 carbons. The nucleophilic moiety can include any suitable nucleophile, e.g., hydrazide, hydroxylamine, semicarbazide, or carbonylhydrazide.

In addition to those described above, other covalent and non-covalent means of attachment may be employed and are well known to those skilled in the art. A tumor-associated protein may be deposited onto a substrate or support by any suitable technique. For example, a tumor-associated protein may be deposited as a monolayer (e.g., a self-assembled monolayer), a continuous layer or as a discontinuous (e.g., patterned) layer. A tumor-associated protein may be deposited or coupled to a support or substrate by modification of the substrate or support by chemical reaction (See, e.g., U.S. Pat. No. 6,444,254), reactive plasma etching, corona discharge treatment, a plasma deposition process, spin coating, dip coating, spray painting, deposition, printing, stamping, diffusion, adsorption/absorption, covalent cross-linking, or combinations thereof. A tumor-associated proteins may be directly spotted onto a surface (e.g., a planar glass surface). In some instances, when necessary or beneficial to keep tumor-associated proteins (e.g., Abs) in a wet environment during the printing process, glycerol (30-40%) may be employed, and/or spotting can be carried out in a humidity-controlled environment.

Antibodies and Autoantibodies

An autoantibody can be an antibody produced by a subject's immune system that is directed against one or more of the subject's own molecules. An autoantibody should be understood to be an antibody, and an antibody may be an autoantibody. In some embodiments, an autoantibody can be directed to one or more of a subject's own proteins. In some embodiments, an autoantibody can bind to one or more of a subject's proteins. In some embodiments, an can autoantibody bind to a tumor-associated protein. An antibody, including an autoantibody, can specifically bind to a particular portion of a protein, including a tumor-associated protein. The portion of a protein to which is an antibody binds may be called an antigenic sequence. A portion of a protein to which an antibody binds may also be referred to as an epitope or an antigenic determinant. An antigenic sequence or epitope may comprise a continuous sequence of amino acids, or it may comprise discontinuous sections of the target protein's amino acid sequence. In some embodiments, an antigenic sequence or epitope of a protein may comprise 1, 2, 3, 4, 5, 6, 7, 8, 9, 10, 11, 12, 13, 14, 15, 16, 17, 18, 19, or 20 amino acids. An antibody can have strong binding affinity for an antigenic sequence or epitope, as measured by its dissociation constant. In some embodiments, an antibody can have dissociation constant of less than about 10⁻⁵M, 10⁻⁶M, 10⁻⁷M, 10⁻⁸M, 10⁻⁹M, 10⁻¹⁰M, 10⁻¹¹M, 10⁻¹²M, 10⁻¹³M, or 10⁻¹⁴M. In some embodiments, an autoantibody can bind to an antigenic sequence having at least about 70%, 75%, 80%, 85%, 90%, 95%, 96%, 97%, 98%, 99%, or 99.5% sequence homology to any one of SEQ ID NOS: 1-21, or a fragment thereof. In some embodiments, an autoantibody can bind to an antigenic sequence as shown in any one of SEQ ID NOS: 22-120 or a fragment thereof. In some embodiments, an autoantibody can bind to an antigenic sequence having at least about 70%, 75%, 80%, 85%, 90%, 95%, 96%, 97%, 98%, 99%, or 99.5% sequence homology to any one of SEQ ID NOS: 22-120, or a fragment thereof. In some embodiments, an autoantibody can be a moiety.

An antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. A naturally occurring antibody can be a protein comprising at least two heavy (H) chains and two light (L) chains inter-connected by disulfide bonds. Each heavy chain can be comprised of a heavy chain variable region (V_{H}) and a heavy chain constant region. The heavy chain constant region can be comprised of three domains, C_{H1}, C_{H2} and C_{H3}. Each light chain can be comprised of a light chain variable region (V_{L}) and a light chain constant region. The light chain constant region can be comprised of one domain, C_{L}. The V_{H} and V_{L} regions can be further subdivided into regions of hypervariability, termed complementary determining regions (CDR), interspersed with regions that are more conserved, termed framework regions (FR). Each V_{H} and V_{L} can be composed of three CDRs and four FRs arranged from amino-terminus to carboxy-terminus in the following order: FR₁, CDR₁, FR₂, CDR₂, FR₃, CDR₃, and FR4. The constant regions of the antibodies may mediate the binding of the immunoglobulin to host tissues or factors, including various cells of the immune system (e.g., effector cells) and the first component (C1 q) of the classical complement system. An antibodies can be of any isotype (e.g., IgG, IgE, IgM, IgD, IgA and IgY), class (e.g., IgG₁, IgG₂, IgG₃, IgG₄, IgA₁ and IgA₂), subclass or modified version thereof. Antibodies may include a complete immunoglobulins or fragments thereof. An antibody fragment can refer to one or more fragments of an antibody that retain the ability to specifically bind to a tumor-associated protein. In addition, aggregates, polymers, and conjugates of immunoglobulins or their fragments can be used where appropriate so long as binding affinity for a particular molecule is maintained. Examples of antibody fragments include a Fab fragment, a monovalent fragment consisting of the V_{L}, V_{H}, C_{L} and C_{H1} domains; a F(ab)₂ fragment, a bivalent fragment comprising two Fab fragments linked by a disulfide bridge at the hinge region; an Fd fragment consisting of the V_{H} and C_{H1} domains; an Fv fragment consisting of the V_{L} and V_{H} domains of a single arm of an antibody; a single domain antibody (dAb) fragment (Ward et al., (1989) Nature 341:544-46), which consists of a V_{H} domain; and an isolated CDR and a single chain Fragment (scFv) in which the V_{L} and V_{H} regions pair to form monovalent molecules (known as single chain Fv (scFv); See, e.g., Bird et al., (1988) Science 242:423-26; and Huston et al., (1988) PNAS 85:5879-83). Thus, antibody fragments include Fab, F(ab)₂, scFv, Fv, dAb, and the like. Although the two domains V_{L} and V_{H} are coded for by separate genes, they can be joined, using recombinant methods, by an artificial peptide linker that enables them to be made as a single protein chain. Such single chain antibodies include one or more antigen binding moieties. These antibody fragments can be obtained using conventional techniques known to those of skill in the art, and the fragments can be screened for utility in the same manner as are intact antibodies. Antibodies can be human, humanized, chimeric, isolated, dog, cat, donkey, sheep, any plant, animal, or mammal. In some aspects, an antibody can be produced by as a result of a disease, disorder, or a condition.

In some embodiments, one or more autoantibody can be associated with a solid support described herein. In some embodiments, one or more autoantibody can be spotted onto a solid support. In other embodiments, one or more autoantibody can be directly or indirectly linked to a solid support described herein.

Complexes

The methods provided herein comprise forming complexes. A complex can refers to an association between at least two moieties (e.g. chemical or biochemical) that have an affinity for one another. The methods provided herein comprise forming a complex between a tumor-associated protein and an antibody, such as an autoantibody. In some embodiments, the methods comprise forming a complex between a tumor-associated protein and a single autoantibody. In some embodiments, the methods comprise forming a complex between a tumor-associated protein and a complex of two or more antibodies. In some embodiments, the methods can comprise forming a complex between a tumor-associated protein and a complex of two or more antibodies. In some embodiments, the methods comprise forming a complex between two or more tumor-associated proteins and a complex of two or more antibodies. In some embodiments, the methods comprise forming a complex between a first complex comprising a tumor-associated protein and another moiety (e.g., a polypeptide, polynucleotide, or small molecule) and an antibody. In some embodiments, the methods comprise forming a complex between a first complex comprising a tumor-associated protein and another moiety (e.g., a polypeptide, polynucleotide, or small molecule) and a second complex comprising two or more antibodies. For example, complexes can be formed between a tumor-associated protein coupled to a solid support, and an antibody to a tumor-associated protein.

Detection Methods

Detection methods for detecting tumor-associated protein-antibody complexes can include photometric and non-photometric means. In some embodiments, such methods process includes a method to detect and measure absorbance, fluorescence, phosphorescence, refractive index, polarization or light scattering. These include direct and/or indirect means to measure such parameters. Methods involving fluorescence include fluorescent tagging in immunological methods such as ELISA or sandwich assay. Methods involving refractive index include surface Plasmon resonance (SPR), grating coupled methods (e.g. sensors uniform grating couplers, wavelength-interrogated optical sensors (WIOS) and chirped grating couplers), resonant minor and interferometric techniques. Methods involving polarization include ellipsometry. Light scattering methods may also be used. Other means for tagging and/or separating and/or detecting can also include magnetic means. Magnetic resonance imaging, gas phase ion spectrometry, MRI may all be used.

Non-photometric methods of detection include, without limitation, magnetic resonance imaging, gas phase ion spectrometry, atomic force microscopy and multipolar coupled resonance spectroscopy. Magnetic resonance imaging (MRI) is based on the principles of nuclear magnetic resonance (NMR), a spectroscopic technique used by scientists to obtain microscopic chemical and physical information about molecules. Gas phase ion spectrometers include mass spectrometers, ion mobility spectrometers and total ion current measuring devices.

Mass spectrometers measure a parameter which can be translated into mass-to-charge ratios of ions. Generally ions of interest bear a single charge, and mass-to-charge ratios are often simply referred to as mass. Mass spectrometers include an inlet system, an ionization source, an ion optic assembly, a mass analyzer, and a detector. Several different ionization sources have been used for desorbing and ionizing analytes from the surface of a support or biochip in a mass spectrometer. Such methodologies include laser desorption/ionization (MALDI, SELDI), fast atom bombardment, plasma desorption, and secondary ion mass spectrometers. In such mass spectrometers the inlet system comprises a support interface capable of engaging the support and positioning it in interrogatable relationship with the ionization source and concurrently in communication with the mass spectrometer, e.g., the ion optic assembly, the mass analyzer and the detector. Solid supports for use in bioassays that have a generally planar surface for the capture of targets and adapted for facile use as supports with detection instruments are generally referred to as biochips.

Analysis of the data generated typically involves quantification of a signal due to the detected antibody versus a control or reference. In some embodiments a control can be a reference and a reference can be a control. The data can be analyzed by any suitable means. Computers and computer programs may be utilized to generate and analyze the data. Beads and/or other supports may be computer coded or coded for identification purposes. Data analysis includes analysis of signal strength under the particular conditions of the assay or detection method. Tumor-associated proteins, antibodies, reference moieties and/or secondary detection moieties may be labeled or radio-labeled or tagged with a detectable moiety. One of ordinary skill in the art can also determine, pursuant to the methods described herein, the presence of false positives or other hits that are or may be found in control samples to account for and/or remove such hits and one of ordinary skill in the art, pursuant to the methods described herein, can continue the process of determining or finding disease associated biomarkers in subject samples having any disease or condition. The detection of such hits, in all cases, can be accomplished by means for detecting the binding of a tumor-associated protein to an antibody.

Binding Assays

Binding assays can also be useful, e.g., for identifying disease related antibodies that interact with the tumor-associated proteins described herein. For example, antibodies or other molecules that bind proteins of the invention can be identified in binding assays. Binding assays can involve, but are not limited to, use of isolated polypeptides, crude extracts, or cell-based assays. In some embodiments the assays described herein can be used to a) identify subjects whose have a first disease or a second disease; (b) assess the impact of a disease therapy; and (c) monitor disease progression.

Binding assays can involve contacting a protein (for example a tumor-associated protein) with a sample comprising an antibody (for example an autoantibody) and allowing sufficient time for the molecule and test agents to form a binding complex. Any binding complexes formed can be detected using any of a number of established analytical techniques. Binding assays include, but are not limited to, methods that measure co-precipitation or co-migration on non-denaturing SDS-polyacrylamide gels, co-migration on Western blots, enzyme linked immunosorbent assay (ELISA), radioimmunoassay (RIA), immunoradiometric assay, fluoroimmunoassay, chemiluminescent assay, bioluminescent assay, FACS, FRET. The steps of various useful immunodetection methods have been described in the scientific literature, such as, e.g., Doolittle and Ben-Zeev (1999), Gulbis and Galand (1993), De Jager et al. (1993), and Nakamura et al. (1987). (see, e.g., Bennet, J. P. and Yamamura, H. I. (1985) “Neurotransmitter, Hormone or Drug Receptor Binding Methods,” in Neurotransmitter Receptor Binding (Yamamura, H. L, et al, eds.), pp. 61-89. Other binding assays involve the use of mass spectrometry or NMR techniques to identify tumor-associated proteins bound the antibody or displacement of labeled substrates. The antibodies used in these assays can be naturally expressed, cloned or synthesized. [108] In addition, mammalian or yeast two-hybrid approaches (see, e.g., Bartel, P. L. et. al. Methods Enzymol, 254:241 (1995)) can be used to identify polypeptides or other molecules that interact or bind to the polypeptide when expressed together in a host cell. U.S. Pat. Nos. 3,817,837, 3,850,752, 3,939,350, 3,996,345, 4,277,437, 4,275,149 and 4,366,241 are hereby incorporated by reference in its entirety.

ELISA

Immunoassays, in their most simple and direct sense, are binding assays. Certain immunoassays finding particular use in the present invention are various types of enzyme linked immunosorbent assays (ELISAs) and radioimmunoassays (RIA) known in the art. In one exemplary ELISA, a tumor-associated protein of the invention can be immobilized onto a selected surface, such as a well in a polystyrene microtiter plate. Then, a test composition suspected of containing the antibody can be added to the wells. After binding and washing to remove non-specifically bound complexes, the bound antibody may be detected. Detection may be achieved by the addition of another ligand linked to a detectable label. This type of assay is analogous to a simple “sandwich ELISA” except that binding of the labeled agent can be direct at the Fab portion of the bound antibody. Detection may also be achieved by the addition of a labeled antibody that binds any bound antibody, e.g., that recognizes the Fc portion of the bound antibody. Optionally, this antibody is not labeled, and is followed by the addition of a second antibody that has binding affinity for the first antibody, with the second antibody being linked to a detectable label.

In another exemplary ELISA, the samples suspected of containing the antibodies can be immobilized onto a well surface and then contacted with labeled tumor-associated proteins of the present invention. After binding and washing to remove non-specifically bound immune complexes, the bound labeled ligands are detected. Alternatively, the ligands may not be labeled and can be detected against an artificial antibody (non-sample) that is selected for specific binding the tumor-associated protein of choice, this second would be linked to a detectable label, thereby permitting detection.

Irrespective of the format employed, ELISAs have certain features in common, such as coating, incubating and binding, washing to remove non-specifically bound species, and detecting the bound immune complexes. These are described below.

In coating a plate with either tumor-associated protein or antibody, one can generally incubate the wells of the plate with a solution of the protein or antibody, either overnight or for a specified period of hours. In certain aspects, the plate can be blocked using a bacterial lysate, such as an E. coli lysate (See Example 1). The wells of the plate can then be washed to remove incompletely adsorbed material. Any remaining available surfaces of the wells can then be coated with a non-specific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface can be contacted with a biological sample or tumor-associated protein to be tested under conditions effective to allow immune complex formation. Detection of the immune complex then requires a labeled secondary binding ligand or antibody, or a secondary binding ligand or antibody in conjunction with a labeled tertiary antibody (with specificity either for the Fc region of the antibody or the ligand).

Under conditions effective to allow immune complex (antigen/antibody) formation means that the conditions can include diluting the antigens and/or antibodies with solutions such as BSA, bovine gamma globulin (BGG) or phosphate buffered saline (PBS)/Tween. These added agents can assist in the reduction of nonspecific background.

The suitable conditions can also mean that the incubation is at a temperature or for a period of time sufficient to allow effective binding. Incubation steps can be from about 1 to 2 to 4 to 6 to 24 to about 48 hours or so, at temperatures on the order of about 20° C. to about 37° C. In some embodiments, about 21° C., 22° C., 23° C., 24° C., 25° C., 26° C., 27° C., 28° C., 29° C., 30° C., 31° C., 32, ° C., 33° C., 34° C., 35° C., 36° C., or about 37° C. or may be overnight at about 2° C., 3° C., 4° C., 5° C., 6° C., 7° C. or so.

Following incubation steps in an ELISA, the contacted surface can be washed so as to remove non-complexed material. In some embodiments, the washing procedure includes washing with a solution such as PBS/Tween, or borate buffer. Following the formation of specific immune complexes between the test sample and the originally bound material, and subsequent washing, the occurrence of even minute amounts of immune complexes may be determined.

Detection may utilize an enzyme that can generate color development upon incubating with an appropriate chromogenic substrate. Thus, for example, one can desire to contact or incubate the first and second immune complex with a urease, glucose oxidase, alkaline phosphatase or hydrogen peroxidase-conjugated antibody or ligand for a period of time and under conditions that favor the development of further immune complex formation (e.g., incubation for about 1, 2, 3, 4, 5, 6, 7, 8, 9, or about 10 hours at room temperature in a PBS-containing solution such as PBS-Tween).

After incubation with a labeled antibody, and subsequent to washing to remove unbound material, the amount of label can be quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label.

Quantification can be achieved by measuring the degree of color generated, e.g., using a visible spectra spectrophotometer.

Förster Resonance Energy Transfer (FRET)

FRET is a phenomenon in which the excited-state energy in one molecule (called the donor) is transferred to another molecule by a radiation less coupling. This mechanism was first correctly described by Förster, and differs from other types of energy transfer, such as electron sharing (Dexter) or trivial transfer (emission of a photon from the donor and reabsorption by the acceptor). The Dexter mechanism requires the two molecules to be in physical contact, while trivial transfer is a very low probability. In contrast, the Förster mechanism exhibits a high probability when the two molecules are within the Förster radius, which is defined for any given pair of fluorophores.

The overall FRET efficiency depends on the Förster radius, and is determined by several factors and is directly related to the amount of overlap between the absorption spectra of the acceptor molecule and the emission spectra of the donor molecule. The amount of FRET also depends on the alignment of the donor and acceptor molecules, although most biological systems are not rigidly aligned. The FRET efficiency is also affected by the ability of the acceptor molecule to absorb light, as indicated by its molar extinction coefficient, and the overall stability of the excited state of the donor molecule, as indicated by the probability that absorption will lead to fluorescence (quantum yield) and the lifetime of the excited state.

FRET between two different fluorophores can be assayed by several methods: looking at the change in color of the fluorescence, measuring the fluorescence lifetime of the donor, examining the changes upon photo bleaching either the donor or acceptor, or by measuring the fluorescence polarization of the acceptor. Regardless of the approach, most of these assays share common features of the instrumentation.

The types of microscopes used to measure FRET can be suitably selected depending on the purpose. In some embodiments, where frequent observations are necessary for monitoring a time course of the changing, conventional incident-light fluorescent microscope can be used. In some embodiments, where resolution is to be increased as in the case where detailed intercellular localization is to be monitored, confocal laser microscope can be used. As a microscope system, an inverted microscope can be used for most live cell measurements in view of keeping the physiological state of cell and preventing contamination. When an upright microscope is used, a water immersion lens can be used in the case of using lens of high power.

The filter set can be suitably selected depending on the fluorescent wave length of the fluorescent protein. For the observation of GFP, a filter with excitation light of about 470-490 nm and fluorescent light of about 500-520 nm can be used. For the observation of YFP, a filter with excitation light of about 490-510 nm and fluorescent light of about 520-550 nm can be used. For the observation of CFP, it is preferred to use a filter with excitation light of about 425 nm and fluorescent light of about 460-500 nm. Moreover, when time course observation is carried out in living cells by using a fluorescent microscope, the cells can be photographed in a short period, and therefore a high sensitive cooled CCD camera can be used. By using a cooled CCD camera, thermal noise can be decreased by cooling CCD, and weak fluorescent image can be clearly acquired by exposure of short period. Confocal microscopes can also be used for live cell imaging, as long as care is taken to minimize the exposure times.

In a similar manner, any ligand may be screened on the beads or supports using the processes described herein. These ligands include, in addition to peptoids or peptides, nucleic acid oligomers, polysaccharides, small molecules and/or any combination thereof which can be built into libraries and, under the conditions recited herein, used to screen biological fluid. In some embodiments, detecting may comprise radio immunoassay (“RIA”), fluorescence immunoassay (“FIA”), enzyme-linked immunosorbent assay (“ELISA”), Western blot, flow cytometry, Forster resonance energy transfer (“FRET”), or surface plasmon resonance.

Methods of Use

The methods, kits, and compositions described herein can be used for numerous applications, including identification of binding partners, determination of affinities of autoantibodies to tumor-associated proteins, determination of specificities of autoantibodies to tumor-associated proteins, quantification of tumor-associated proteins in a sample, quantification of autoantibodies in a sample, quantification of binding events, identification of a disease, identification of biomarkers of a disease or condition, drug discovery, molecular biology, immunology and toxicology. Arrays can be used for large scale binding assays in numerous diagnostic and screening applications. These methods of use include, but are not limited to, high-content, high-throughput assays for screening for antibodies that interact with tumor-associated proteins. Additional methods of use include medical diagnostic, proteomic, and biosensor assays. The multiplexed measurement of quantitative variation in levels of large numbers of tumor-associated proteins allows the recognition of patterns defined by several to many different tumor-associated proteins. The multiplexed identification of large numbers of interactions between tumor-associated proteins and autoantibodies allows for the recognition of binding and interaction patterns defined by several to many different interactions between tumor-associated proteins and autoantibodies.

The assays used with the arrays of the presently disclosed subject matter may be direct, noncompetitive assays or indirect, competitive assays. In the noncompetitive method, the affinity for a tumor-associated protein to an antibody can be determined directly. In this method, the tumor-associated protein can be directly exposed to an antibody. The antibody may be labeled or unlabeled.

A label refers to a molecule that, when attached to another molecule provides or enhances a means of detecting the other molecule. A signal emitted from a label can allow detection of the molecule or complex to which it is attached, and/or the label itself. For example, a label can be a molecular species that elicits a physical or chemical response that can be observed or detected by the naked eye or by means of instrumentation such as, without limitation, scintillation counters, colorimeters, UV spectrophotometers and the like. Labels include but are not limited to, radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal generation detection methods include: chemiluminescence detection, electrochemiluminescence detection, Raman energy detection, colorimetric detection, hybridization protection assays, and mass spectrometry.

If an antibody is labeled, the methods of detection could include fluorescence, phosphorescence luminescence, radioactivity, and the like. If an antibody is unlabeled, the detection of binding can be based on a change in some physical property of the tumor-associated protein. Such physical properties could include, for example, a refractive index or electrical impedance. The detection of binding of unlabeled antibody could include, for example, mass spectroscopy. The detection of binding can also be carried out using sandwich assays, in which after the initial binding, the array is incubated with a second solution containing molecules such as labeled antibodies that have an affinity for the autoantibody bound to the tumor-associated protein, and the amount of binding is determined based on the amount of binding of the labeled antibodies to the autoantibody. The detection of binding can be carried out using a displacement assay in which after the initial binding of a labeled moiety, the array is incubated with a second solution containing unlabeled binding moiety. The binding capability and the amount of binding of the binding moiety are determined based on the decrease in number of the pre-bound labeled moieties in the tumor-associated proteins.

Also disclosed herein is a method of determining a relative binding affinity of an antibody for a tumor-associated protein.

The relative binding affinity of an antibody for a tumor-associated protein may be measured by measuring or counting the coupled product and/or amplified products thereof by using any suitable method known in the art.

A variety of different agents may be screened by the above methods. Candidate agents encompass numerous chemical classes including, but not limited to, peptides, polynucleotides, and organic molecules (e.g., small organic compounds having a molecular weight of more than 50 and less than about 2,500 Daltons). Candidate agents can comprise functional groups for structural interaction with tumor-associated proteins, such as hydrogen bonding, and can include at least one or at least two of an amine, carbonyl, hydroxyl or carboxyl group. The candidate agents can comprise cyclical carbon or heterocyclic structures and/or aromatic or polyaromatic structures substituted with one or more functional groups. Candidate agents can be biomolecules including peptides, saccharides, fatty acids, steroids, purines, pyrimidines, derivatives, structural analogs or combinations thereof. Candidate agents can be obtained from a wide variety of sources including libraries of synthetic or natural compounds. For example, numerous means are available for random and directed synthesis of a wide variety of organic compounds and biomolecules, including expression of randomized polynucleotides and polypeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, acidification, etc. to produce structural analogs. Agents identified find can uses in a variety of methods, including methods of modulating the activity of a tumor-associated protein, and conditions related to the presence, activity, and/or interactions of a tumor-associated protein. In some embodiments, screening can be performed by exposing a solid support comprising one or more antibody, autoantibody, or tumor-associated protein with one or more agent.

Diagnostics

The methods and apparatus disclosed herein can be used to screen for various diseases or conditions, including an alteration in the state of the body or of some of the organs, interrupting or disturbing the performance of the functions and/or causing symptoms such as discomfort, dysfunction, distress, or even death to the person afflicted or those in contact with a person. A disease or condition can also include a distemper, ailing, ailment, malady, disorder, sickness, illness, complain, interdisposition and/or affectation.

For example, samples containing antibodies from a diseased animal can be simultaneously screened for the antibodies' ability to interact with tumor-associated proteins on an array. These interactions can be compared to those of samples from individuals that are not in a disease state, not presenting symptoms of persons in the disease state, or presenting symptoms of the disease state. For example, the levels of tumor-associated proteins in samples from a diseased animal can be simultaneously determined. These levels can be compared to those of samples from individuals that are not in a disease state, not presenting symptoms of persons in the disease state, or presenting symptoms of the disease state.

The methods, kits, and compositions described herein can be used in medical diagnostics, drug discovery, molecular biology, immunology and toxicology. Arrays can be used for large scale binding assays in numerous diagnostic and screening applications. The multiplexed measurement of quantitative variation in levels of large numbers of tumor-associated proteins (e.g. proteins) allows the recognition of patterns defined by several to many different tumor-associated proteins. The multiplexed identification of large numbers of interactions between tumor-associated proteins and antibodies allows for the recognition of binding and interaction patterns defined by several to many different interactions between tumor-associated proteins and antibodies. Many physiological parameters and disease-specific patterns can be simultaneously assessed. One embodiment involves the separation, identification and characterization of proteins present in a biological sample. For example, by comparison of disease and control samples, it can be possible to identify disease specific tumor-associated proteins. These tumor-associated proteins can be used as targets for drug development or as molecular markers of disease.

Detection a level of one or more tumor-associated proteins or detection of interactions between autoantibodies and tumor-associated proteins can lead to a medical diagnosis. The sample can be a sample from a subject with a condition or disease. For example, a sample can be a diseased tissue or cell, such as a breast cancer, ovarian cancer, lung cancer (including SCLC or NSCLC), colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer tissue or cell. The sample can be from a subject with a disease or condition such as a cancer, inflammatory disease, immune disease, autoimmune disease, cardiovascular disease, neurological disease, infectious disease, metabolic disease, or a perinatal condition. For example, the disease or condition can be a tumor, neoplasm, or cancer. The cancer can be, but is not limited to, breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer. The colorectal cancer can be CRC Dukes B or Dukes C-D. The hematological malignancy can be B-Cell Chronic Lymphocytic Leukemia, B-Cell Lymphoma-DLBCL, B-Cell Lymphoma-DLBCL-germinal center-like, B-Cell Lymphoma-DLBCL-activated B-cell-like, or Burkitt's lymphoma. The disease or condition can also be a premalignant condition, such as Barrett's Esophagus. The disease or condition can also be an inflammatory disease, immune disease, or autoimmune disease. For example, the disease may be inflammatory bowel disease (IBD), Crohn's disease (CD), ulcerative colitis (UC), pelvic inflammation, vasculitis, psoriasis, diabetes, autoimmune hepatitis, Multiple Sclerosis, Myasthenia Gravis, Type I diabetes, Rheumatoid Arthritis, Psoriasis, Systemic Lupus Erythematosis (SLE), Hashimoto's Thyroiditis, Grave's disease, Ankylosing Spondylitis Sjogrens Disease, CREST syndrome, Scleroderma, Rheumatic Disease, organ rejection, Primary Sclerosing Cholangitis, or sepsis. The disease or condition can also be a cardiovascular disease, such as atherosclerosis, congestive heart failure, vulnerable plaque, stroke, or ischemia. The cardiovascular disease or condition can be high blood pressure, stenosis, vessel occlusion or a thrombotic event. The disease or condition can also be a neurological disease, such as Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neuropsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, or chronic fatigue syndrome. The condition may also be fibromyalgia, chronic neuropathic pain, or peripheral neuropathic pain. The disease or condition may also be an infectious disease, such as a bacterial, viral or yeast infection. For example, the disease or condition may be Whipple's Disease, Prion Disease, cirrhosis, methicillin-resistant Staphylococcus aureus, HIV, hepatitis, syphilis, meningitis, malaria, tuberculosis, or influenza. The disease or condition can also be a perinatal or pregnancy related condition (e.g. preeclampsia or preterm birth), zika virus, dengue fevor, flavivirus, or a metabolic disease or condition, such as a metabolic disease or condition associated with iron metabolism.

Specificity and Sensitivity

Methods herein can further comprise determining the effectiveness of a given biomarker (e.g., tumor-associated protein or antibody) or a given group of biomarkers. Parameters to be measured include those described in Fischer et al., Intensive Care Med. 29: 1043-51, 2003, which is incorporated herein in its entirety. These parameters include sensitivity and specificity, predictive values, likelihood ratios, diagnostic odds ratios, and receiver operating characteristic (ROC) curve areas. One or a group of effective biomarkers can exhibit one or more of the following results on these various parameters: at least 75% sensitivity, combined with at least 75% specificity; ROC curve area of at least 0.7, at least 0.8, at least 0.9, or at least 0.95; and/or a positive likelihood ratio (calculated as sensitivity/(1-specificity)) of at least 5, at least 10, or at least 20, and a negative likelihood ratio (calculated as (1-sensitivity)/specificity) of less than or equal to 0.3, less than or equal to 0.2, or less than or equal to 0.1. The ROC areas can be calculated and used in determining the effectiveness of a biomarker as described in US Patent Application Publication No. 2013/0189243, which is incorporated herein in its entirety.

Methods, devices and kits provided herein can assess a condition (e.g., cancer) in a subject with high specificity and sensitivity. In some embodiments, the cancer can be lung cancer. As used herein, the term “specificity” can refer to a measure of the proportion of negatives that are correctly identified as such (e.g., the percentage of healthy people who are correctly identified as not having the condition). As used herein, the term “sensitivity” can refer to a measure of the proportion of positives that are correctly identified as such (e.g., the percentage of sick people who are correctly identified as having the condition). Methods, devices and kits provided herein can assess a condition in a subject with a specificity of at least about 75%, 80%, 85%, 90%, 95%, 96%, 97%, 98%, 99%, or 100%. Methods, devices and kits provided herein can assess a condition in a subject with a sensitivity of at least about 10%, 15%, 20%, 25%, 30%, 35%, 40%, 45%, 50%, 55%, 60%, 70%, 75%, 80%, 85%, 90%, 95%, 96%, 97%, 98%, 99%, or 100%. Methods, devices and kits provided herein can assess a condition in a subject with a specificity of at least about 70% and a sensitivity of at least about 70%, a specificity of at least about 75% and a sensitivity of at least about 75%, a specificity of at least about 80% and a sensitivity of at least about 80%, a specificity of at least about 85% and a sensitivity of at least about 85%, a specificity of at least about 90% and a sensitivity of at least about 90%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of about 100% a sensitivity of about 100%.

Methods of assessing a condition in a subject herein can achieve high specificity and sensitivity based on the expression of various numbers of biomarkers. In some cases, the methods of assessing a condition in a subject can achieve a specificity of at least about 70% and a sensitivity of at least about 70%, a specificity of at least about 75% and a sensitivity of at least about 75%, a specificity of at least about 80% and a sensitivity of at least about 80%, a specificity of at least about 85% and a sensitivity of at least about 85%, a specificity of at least about 90% and a sensitivity of at least about 90%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of 100% a sensitivity of 100% based on the expression of no more than 2, 3, 4, 5, 6, 7, 8, 9, or 10 biomarkers. In some cases, the methods, devices and kits of assessing a condition in a subject can achieve a specificity of at least about 92% and a sensitivity of at least about 92%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of about 100% and a sensitivity of about 100% based on the expression of two biomarkers. In some cases, the methods of assessing a condition in a subject can comprise measuring or detecting the expression of two or more of any antibody or tumor-associated protein disclosed herein for example (p53, ETHE1, CTAG1A, C1QTNF1, TEX264, CLDN2, NSG1, HRAs, CKAP2, DPP4, CAB39, or STRA13, LIMS1, RBPJ1, RBPJ2, RBPJ3, MAK16, CACLOCO2, SDCBP, LGALS8, DMRT2 or a fragment of any of the above) and the method can achieve a specificity of at least 50% and a sensitivity of at least 50%, a specificity of at least 55% and a sensitivity of at least 55%, a specificity of at least 60% and a sensitivity of at least 60%, a specificity of at least 65% and a sensitivity of at least 65%, a specificity of at least 70% and a sensitivity of at least 70%, a specificity of at least 75% and a sensitivity of at least 75%, a specificity of at least 80% and a sensitivity of at least 80%, a specificity of at least 85% and a sensitivity of at least 85%, a specificity of at least 90% and a sensitivity of at least 90%, a specificity of at least 92% and a sensitivity of at least 92%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% and a sensitivity of 100%.

Assessing lung cancer can comprise distinguishing a subject with lung cancer from a healthy subject, or a subject with a second disease. In some embodiments, a second disease can be a second cancer. In some embodiments, a second disease can be a non-cancer pulmonary disease. In some embodiments, a non-cancer pulmonary disease can be benign lung lesions (LBL), pneumonia, chronic obstructive pulmonary disease (COPD), or pulmonary tuberculosis. Methods, devices, and kits herein can achieve high specificity and sensitivity in distinguishing a subject with a disease disclosed herein for example lung cancer from a healthy subject, and distinguishing the subject with lung cancer from a subject with a second disease. For example, methods, devices, and kits herein can achieve a specificity of at least 92% and a sensitivity of at least 92%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% and a sensitivity of 100% in distinguishing a subject with lung cancer from a healthy subject, and meanwhile can achieve a specificity of at least 92% and a sensitivity of at least 92%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% and a sensitivity of 100% in distinguishing the subject with lung cancer from a subject with a second disease.

Supports/Substrates

The present disclosure provides substrates and methods of making substrates. The nature and geometry of a support or substrate can depend upon a variety of factors, including the type of array (e.g., one-dimensional, two-dimensional or three-dimensional). Generally, a substrate can be composed of any material which will permit coupling of a tumor-associated protein, which will not melt or otherwise substantially degrade under the conditions used to hybridize and/or denature nucleic acids. A substrate can be composed of any material which will permit coupling of a tumor-associated protein, and/or other moiety at one or more discrete regions and/or discrete locations within the discrete regions. A substrate can be composed of any material which permit washing or physical or chemical manipulation without dislodging a tumor-associated protein or antibody from the solid support.

Substrates can be fabricated by the transfer of tumor-associated protein onto the solid surface in an organized high-density format followed by coupling the tumor-associated protein thereto. The techniques for fabrication of a substrate of the invention include, but are not limited to, photolithography, ink jet and contact printing, liquid dispensing and piezoelectrics. The patterns and dimensions of arrays are to be determined by each specific application. The sizes of each tumor-associated protein spots may be easily controlled by the users.

A method of making a solid substrate can comprise contacting or coupling a tumor-associated protein to a discrete location.

A substrate may take a variety of configurations ranging from simple to complex, depending on the intended use of the array. Thus, a substrate can have an overall slide or plate configuration, such as a rectangular or disc configuration. A standard microplate configuration can be used. In some embodiments, the surface may be smooth or substantially planar, or have irregularities, such as depressions or elevations. For example, the substrates of the presently disclosed subject matter can include at least one surface on which a pattern of recombinant virion microspots can be coupled or deposited. In some instances, a substrate may have a rectangular cross-sectional shape, having a length of from about 10-200 mm, 40-150 mm, or 75-125 mm; a width of from about 10-200 mm, 20-120 mm, or 25-80 mm, and a thickness of from about 0.01-5.0 mm, 0.1-2 mm, or 0.2 to 1 mm.

A support may be organic or inorganic; may be metal (e.g., copper or silver) or non-metal; may be a polymer or nonpolymer; may be conducting, semiconducting or nonconducting (insulating); may be reflecting or nonreflecting; may be porous or nonporous; etc. A solid support as described above can be formed of any suitable material, including metals, metal oxides, semiconductors, polymers (particularly organic polymers in any suitable form including woven, nonwoven, molded, extruded, cast, etc.), silicon, silicon oxide, and composites thereof.

A number of materials (e.g., polymers) suitable for use as substrates (e.g., solid substrates) in the instant invention have been described in the art. Suitable materials for use as substrates include, but are not limited to, polycarbonate, gold, silicon, silicon oxide, silicon oxynitride, indium, tantalum oxide, niobium oxide, titanium, titanium oxide, platinum, iridium, indium tin oxide, diamond or diamond-like film, acrylic, styrene-methyl methacrylate copolymers, ethylene/acrylic acid, acrylonitrile-butadiene-styrene (ABS), ABS/polycarbonate, ABS/polysulfone, ABS/polyvinyl chloride, ethylene propylene, ethylene vinyl acetate (EVA), nitrocellulose, nylons (including nylon 6, nylon 6/6, nylon 6/6-6, nylon 6/9, nylon 6/10, nylon 6/12, nylon 11 and nylon 12), polyacrylonitrile (PAN), polyacrylate, polycarbonate, polybutylene terephthalate (PBT), poly(ethylene) (PE) (including low density, linear low density, high density, cross-linked and ultra-high molecular weight grades), poly(propylene) (PP), cis and trans isomers of poly(butadiene) (PB), cis and trans isomers of poly(isoprene), polyethylene terephthalate) (PET), polypropylene homopolymer, polypropylene copolymers, polystyrene (PS) (including general purpose and high impact grades), polycarbonate (PC), poly(epsilon-caprolactone) (PECL or PCL), poly(methyl methacrylate) (PMMA) and its homologs, poly(methyl acrylate) and its homologs, poly(lactic acid) (PLA), poly(glycolic acid), polyorthoesters, poly(anhydrides), nylon, polyimides, polydimethylsiloxane (PDMS), polybutadiene (PB), polyvinylalcohol (PVA), polyacrylamide and its homologs such as poly(N-isopropyl acrylamide), fluorinated polyacrylate (PFOA), poly(ethylene-butylene) (PEB), poly(styrene-acrylonitrile) (SAN), polytetrafluoroethylene (PTFE) and its derivatives, polyolefin plastomers, fluorinated ethylene-propylene (FEP), ethylene-tetrafluoroethylene (ETFE), perfluoroalkoxyethylene (PFA), polyvinyl fluoride (PVF), polyvinylidene fluoride (PVDF), polychlorotrifluoroethylene (PCTFE), polyethylene-chlorotrifluoroethylene (ECTFE), styrene maleic anhydride (SMA), metal oxides, glass, silicon oxide or other inorganic or semiconductor material (e.g., silicon nitride), compound semiconductors (e.g., gallium arsenide, and indium gallium arsenide), and combinations thereof.

Examples of well-known solid supports include polypropylene, polystyrene, polyethylene, dextran, nylon, amylases, glass, natural and modified celluloses (e.g., nitrocellulose), polyacrylamides, agaroses and magnetite. In some instances, the solid support can be silica or glass because of its great chemical resistance against solvents, its mechanical stability, its low intrinsic fluorescence properties, and its flexibility of being readily functionalized. In one embodiment, the substrate is glass, particularly glass coated with nitrocellulose, more particularly a nitrocellulose-coated slide (e.g., FAST slides).

A substrate may be modified with one or more different layers of compounds or coatings that serve to modify the properties of the surface in a desirable manner. For example, a substrate may further comprise a coating material on the whole or a portion of the surface of the substrate. In some embodiments, a coating material enhances the affinity of the tumor-associated protein or another moiety (e.g., a functional group) for the substrate. For example, the coating material can be nitrocellulose, silane, thiol, disulfide, or a polymer. When the material is a thiol, the substrate may comprise a gold-coated surface and/or the thiol comprises hydrophobic and hydrophilic moieties. When the coating material is a silane, the substrate comprises glass and the silane may present terminal moieties including, for example, hydroxyl, carboxyl, phosphate, glycidoxy, sulfonate, isocyanato, thiol, or amino groups. In an alternative embodiment, the coating material may be a derivatized monolayer or multilayer having covalently bonded linker moieties. For example, the monolayer coating may have thiol (e.g., a thioalkyl selected from the group consisting of a thioalkyl acid (e.g., 16-mercaptohexadecanoic acid), thioalkyl alcohol, thioalkyl amine, and halogen containing thioalkyl compound), disulfide or silane groups that produce a chemical or physicochemical bonding to the substrate. The attachment of the monolayer to the substrate may also be achieved by non-covalent interactions or by covalent reactions.

After attachment to the substrate, a coating may comprise at least one functional group. Examples of functional groups on the monolayer coating include, but are not limited to, carboxyl, isocyanate, halogen, amine or hydroxyl groups. In one embodiment, these reactive functional groups on the coating may be activated by standard chemical techniques to corresponding activated functional groups on the monolayer coating (e.g., conversion of carboxyl groups to anhydrides or acid halides, etc.). Exemplary activated functional groups of the coating on the substrate for covalent coupling to terminal amino groups include anhydrides, N-hydroxysuccinimide esters or other common activated esters or acid halides, Exemplary activated functional groups of the coating on the substrate include anhydride derivatives for coupling with a terminal hydroxyl group; hydrazine derivatives for coupling onto oxidized sugar residues of the linker compound; or maleimide derivatives for covalent attachment to thiol groups of the linker compound. To produce a derivatized coating, at least one terminal carboxyl group on the coating can be activated to an anhydride group and then reacted, for example, with a linker compound. Alternatively, the functional groups on the coating may be reacted with a linker having activated functional groups (e.g., N-hydroxysuccinimide esters, acid halides, anhydrides, and isocyanates) for covalent coupling to reactive amino groups on the coating.

A substrate can contain a linker (e.g., to indirectly couple a moiety to the substrate). In one embodiment, a linker has one terminal functional group, a spacer region and a tumor-associated protein adhering region. The terminal functional groups for reacting with functional groups on an activated coating include halogen, amino, hydroxyl, or thiol groups. In some instances, a terminal functional group is selected from the group consisting of a carboxylic acid, halogen, amine, thiol, alkene, acrylate, anhydride, ester, acid halide, isocyanate, hydrazine, maleimide and hydroxyl group. The spacer region may include, but is not limited to, polyethers, polypeptides, polyamides, polyamines, polyesters, polysaccharides, polyols, multiple charged species or any other combinations thereof. Exemplary spacer regions include polymers of ethylene glycols, peptides, glycerol, ethanolamine, serine, inositol, etc. The spacer region may be hydrophilic in nature. The spacer region may be hydrophobic in nature. In some instances, the spacer has n oxyethylene groups, where n is between 2 and 25. In some instances, a region of a linker that adheres to a tumor-associated protein, or other moiety is hydrophobic or amphiphilic with straight or branched chain alkyl, alkynyl, alkenyl, aryl, arylalkyl, heteroalkyl, heteroalkynyl, heteroalkenyl, heteroaryl, or heteroarylalkyl. In some instances, a region of a linker that adheres to a tumor-associated protein, or other moiety comprises a C₁₀-C₂₅ straight or branched chain alkyl or heteroalkyl hydrophobic tail. In some instances, a linker comprises a terminal functional group on one end, a spacer, a tumor-associated protein adhering region, and a hydrophilic group on another end. The hydrophilic group at one end of the linker may be a single group or a straight or branched chain of multiple hydrophilic groups (e.g., a single hydroxyl group or a chain of multiple ethylene glycol units).

In some embodiments, a support can be planar. In some instances, a support can be spherical. In some instances, a support can be a bead. In some instances, a support can be magnetic. In some instances, a magnetic solid support can comprises magnetite, maghemitite, FePt, SrFe, iron, cobalt, nickel, chromium dioxide, ferrites, or mixtures thereof. In some instances, a support can be nonmagnetic. In some embodiments, the nonmagnetic solid support can comprise a polymer, metal, glass, alloy, mineral, or mixture thereof. In some instances a nonmagnetic material can be a coating around a magnetic solid support. In some instances, a magnetic material may be distributed in the continuous phase of a magnetic material. In some embodiments, the solid support comprises magnetic and nonmagnetic materials. In some instances, a solid support can comprise a combination of a magnetic material and a nonmagnetic material. In some embodiments, the magnetic material is at least about 5, 10, 20, 30, 40, 50, 60, 70, or about 80% by weight of the total composition of the solid support. In some embodiments, the bead size can be quite large, on the order of 100-900 microns or in some cases even up to a diameter of 3 mm. In other embodiments, the bead size can be on the order of 1-150 microns. The average particle diameters of beads of the invention can be in the range of about 2 μm to several millimeters, e.g., diameters in ranges having lower limits of 2 μm, 4 μm, 6 μm, 8 μm, 10 μm, 20 μm, 30 μm, 40 μm, 50 μm, 60 μm, 70 μm, 80 μm, 90 μm, 100 μm, 150 μm, 200 μm, 300 μm, or 500 μm, and upper limits of 20 μm, 30 μm, 40 μm, 50 μm, 60 μm, 70 μm, 80 μm, 90 μm, 100 μm, 150 μm, 200 μm, 300 μm, 500 μm, 750 μm, 1 mm, 2 mm, or 3 mm.

A support or substrate can be an array. In some embodiment a solid support comprises an array. An array of the invention can comprise an ordered spatial arrangement of two or more discrete regions. Address, spot, microspot, and discrete region are terms used interchangeably and refer to a particular position, such as on an array. An array can comprise tumor-associated proteins located at known or unknown discrete regions. An array can comprise tumor-associated proteins or antibodies located at known or unknown discrete regions.

Each of two or more discrete regions can comprise a tumor-associated protein. Each of two or more discrete regions can comprise a tumor-associated protein. The two or more discrete regions of an array can comprise two or more first discrete locations and two or more second discrete locations.

For example, an array can comprise a first discrete region comprising a first tumor-associated protein, and a second discrete region comprising a second tumor-associated protein.

Row and column arrangements of arrays can be selected due to the relative simplicity in making such arrangements. The spatial arrangement can, however, be essentially any form selected by the user, and optionally, in a pattern. Microspots of an array may be any convenient shape, including circular, ellipsoid, oval, annular, or some other analogously curved shape, where the shape may, in certain embodiments, be a result of the particular method employed to produce the array. The microspots may be arranged in any convenient pattern across or over the surface of the array, such as in rows and columns so as to form a grid, in a circular pattern, and the like, where generally the pattern of spots will be present in the form of a grid across the surface of the substrate.

An array can comprise an ordered spatial arrangement of two or more tumor-associated proteins, on a solid surface. For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 tumor-associated proteins. An array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 antibodies specific for a tumor-associated protein. The tumor-associated proteins can be linked to the array by the antibodies. Thus, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 tumor-associated proteins linked to the array by at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 antibodies specific for the tumor-associated proteins. In some embodiments, a solid support can comprise not more than 1000 tumor-associated proteins. In some embodiments, a solid support can comprise not more than 100 tumor-associated proteins. In some embodiments, a solid support can comprise not more than 10 tumor-associated proteins. In some embodiments, a solid support can comprise not more than 1 tumor-associated proteins. In some embodiments, a solid support can comprise not more than 2 tumor-associated proteins. In some embodiments, a solid support can comprise not more than 3 tumor-associated proteins. In some embodiments, a solid support can comprise not more than 3 tumor-associated proteins. In some embodiments, a solid support can comprise not more than 4 tumor-associated proteins. In some embodiments, a solid support can comprise not more than 5 tumor-associated proteins.

An array can comprise an ordered spatial arrangement of two or more same or different tumor-associated proteins, on a solid surface. For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 same or different tumor-associated proteins. For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 same or different tumor-associated proteins.

An array can be a high-density array. A high-density array can comprise tens, hundreds, thousands, tens-of-thousands or hundreds-of-thousands of tumor-associated proteins. The density of microspots of an array may be at least about 1/cm² or at least about 10/cm², up to about 1,000/cm² or up to about 500/cm². In certain embodiments, the density of all the microspots on the surface of the substrate may be up to about 400/cm², up to about 300/cm², up to about 200/cm², up to about 100/cm², up to about 90/cm², up to about 80/cm², up to about 70/cm², up to about 60/cm², or up to about 50/cm². For example, an array can comprise at least 50, 60, 70, 80, 90, 100, 150, 200, 250, 300, 350, 400, 450, 500, 600, 700, 800, 900, or 1,000 distinct antibodies per a surface area of less than about 1 cm². For example, an array can comprise 50, 60, 70, 80, 90, 100, 150, 200, 250, 300, 350 or 400 discrete regions in an area of about 16 mm², or 2,500 discrete regions/cm². In some embodiments, tumor-associated proteins, linkers, or another moiety in each discrete region can be present in a defined amount (e.g., between about 0.1 femtomoles and 100 nanomoles). For example, an array can comprise at least about 2 tumor-associated proteins per cm². For example, an array can comprise at least about 2, 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, or more tumor-associated proteins. For example, an array can be a high-density protein array comprising at least about 10 tumor-associated proteins per cm². For example, an array can comprise at least about 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, or more tumor-associated proteins per cm².

Kits

Also provided are kits that find use in practicing the subject methods, as mentioned above. A kit can include one or more of the compositions described herein. A kit can include at least one tumor-associated protein. A kit can include at least one antibody.

A kit can include a solid support. In some embodiments, a solid support is already functionalized with at least one tumor-associated protein. In some embodiments, a solid support is not functionalized with at least one tumor-associated protein. A kit can include a reagent for coupling at least one tumor-associated protein to the solid support.

A kit can include one or more reagents for performing amplification, including suitable primers, enzymes, nucleobases, and other reagents such as PCR amplification reagents (e.g., nucleotides, buffers, cations, etc.), and the like. Additional reagents that are required or desired in the protocol to be practiced with the kit components may be present. Such additional reagents include, but are not limited to, one or more of the following an enzyme or combination of enzymes such as a polymerase, reverse transcriptase, nickase, restriction endonuclease, uracil-DNA glycosylase enzyme, enzyme that methylates or demethylates DNA, endonuclease, ligase, etc.

As indicated above, certain protocols will employ two or more different sets of such probes for simultaneous detection of two or more tumor-associated proteins in a sample (e.g., in multiplex and/or high throughput formats). In some embodiments a kit includes two or more distinct sets of antibodies, and/or tumor-associated proteins.

The kit components may be present in separate containers, or one or more of the components may be present in the same container, where the containers may be storage containers and/or containers that are employed during the assay for which the kit is designed.

In addition to the above components, the subject kits may further include instructions for practicing the subject methods. These instructions may be present in the subject kits in a variety of forms, such as printed information on a suitable medium or substrate (e.g., a piece or pieces of paper on which the information is printed), in the packaging of the kit, in a package insert, etc. Yet another means would be a computer readable medium (e.g., diskette, CD, etc.), on which the information has been recorded. Yet another means that may be present is a website address which may be used via the internet to access the information at a removed site.

Communicating a Result

Additional embodiments of the invention relate to the communication of assay results or diagnoses or both to technicians, physicians or subjects, for example. In certain embodiments, computers can be used to communicate results of the assessing or diagnoses or both to interested parties, e.g., physicians and their subjects. In some embodiments, the assessing can be performed or results analyzed in a country or jurisdiction which differs from the country or jurisdiction to which the results or diagnoses are communicated. In some embodiments of the invention, a diagnosis based on the presence or absence in a test subject of any biomarker identified by the invention may be communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test maybe generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions

### Other Embodiments

The section headings used herein are for organizational purposes only and are not to be construed as limiting the subject matter described.

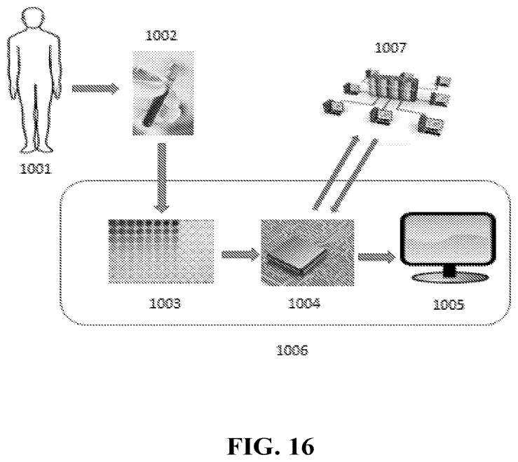

FIG. 16 shows an exemplary method for assessing LC or a disease or condition described herein in a subject. Peripheral blood (FIG. 16, 1002) can be drawn from a subject (FIG. 16, 1001). The expression of a group of biomarkers (for example autoantibodies) in the blood can be measured by an assay (FIG. 16, 1003). In some cases, the assay can be a protein-based assay, such as enzyme-linked immunosorbent assay (ELISA). In some cases, a autoantibody expression can be measured for assessing LC or a disease or condition described herein. The expression levels or signal intensity autoantibodies can be analyzed by a computer system (FIG. 16, 1004). In some cases, the computer system can compare the expression or signal of the autoantibodies to a reference. The reference can be stored in the computer system. Alternatively, the reference can be stored in other computers, databases, and/or servers, and accessible through a network (e.g. Internet) (FIG. 16, 1007). The result of whether a subject has LC, a disease or condition described herein can be transmitted to an output device, e.g., a monitor (FIG. 16, 1005). The assay, the computer system, and the output device (FIGS. 16, 1003, 1004 and 1005) can be integrated into a single device (FIG. 16, 1006). In some cases, such device can be a point of care device, e.g., a portable point of care device. In some cases, the computer system can be a smartphone.

It is to be understood that the methods and compositions described herein are not limited to the particular methodology, protocols, constructs, and reagents described herein and as such may vary. It is also to be understood that the terminology used herein is for the purpose of describing particular embodiments only, and is not intended to limit the scope of the methods and compositions described herein, which will be limited only by the appended claims. While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXAMPLES

### Example 1. Collection of Serum Samples

Serum samples were collected at Fujian Provincial Hospital during a period between 2014 and 2016. This cohort was comprised of 1101 serum samples collected from 560 LC patients, 162 healthy persons, 153 patients with lung benign lesions (LBL), including 83 pneumonia, 39 chronic obstructive pulmonary disease (COPD) and 31 pulmonary tuberculosis (TB), and 226 patients with other cancers, including rectal cancer (RC), liver cancer (LiC), cervical cancer (CC), esophagus cancer (EC), and gastric cancer (GC). Serum samples were obtained by separation from peripheral blood and stored at −80° C. until use. Table 1 discloses characteristics of all samples used in this study.

TABLE 1
Characteristics of serum samples

| | | | | | | | <20 | |
|---|---|---|---|---|---|---|---|---|
| | | | | | | | pack | |
| # | Age | Sex | Type | TNM class | Stage | Smoker | years | Cohort |
| 1 | 56 | Male | Healthy | | | ✓ | ✓ | HuProt Array |
| 2 | 62 | Female | Healthy | | | | | HuProt Array |
| 3 | 58 | Male | Healthy | | | ✓ | | HuProt Array |
| 4 | 35 | Female | Healthy | | | | | HuProt Array |
| 5 | 44 | Male | Healthy | | | ✓ | | HuProt Array |
| 6 | 70 | Male | Healthy | | | ✓ | | HuProt Array |
| 7 | 55 | Male | Healthy | | | ✓ | | HuProt Array |
| 8 | 41 | Male | Healthy | | | ✓ | ✓ | HuProt Array |
| 9 | 69 | Female | Healthy | | | ✓ | | HuProt Array |
| 10 | 64 | Male | Healthy | | | ✓ | | HuProt Array |
| 11 | 51 | Male | Healthy | | | ✓ | ✓ | HuProt Array |
| 12 | 41 | Male | Healthy | | | ✓ | ✓ | HuProt Array |
| 13 | 55 | Male | Healthy | | | ✓ | ✓ | HuProt Array |
| 14 | 72 | Female | Healthy | | | | | HuProt Array |
| 15 | 63 | Male | Healthy | | | ✓ | ✓ | HuProt Array |
| 16 | 73 | Male | Healthy | | | ✓ | | HuProt Array |
| 17 | 58 | Male | Healthy | | | ✓ | | HuProt Array |
| 18 | 43 | Male | Healthy | | | ✓ | | HuProt Array |
| 19 | 63 | Female | Healthy | | | | | HuProt Array |
| 20 | 55 | Female | Healthy | | | | | HuProt Array |
| 21 | 78 | Female | Large Cell LC | T4N2M1b | IV | ✓ | | HuProt Array |
| 22 | 53 | Male | Large Cell LC | T2aN2M0 | IIIa | ✓ | | HuProt Array |
| 23 | 55 | Female | Large Cell LC | T2aN2M0 | IIIa | | | HuProt Array |
| 24 | 58 | Male | Large Cell LC | T1bN2M0 | IIIa | ✓ | | HuProt Array |
| 25 | 50 | Male | Large Cell LC | T4N2M0 | IIIb | | | HuProt Array |
| 26 | 53 | Female | Large Cell LC | T2aN3M0 | IIIb | | | HuProt Array |
| 27 | 68 | Male | Large Cell LC | T2aN0M0 | Ib | ✓ | | HuProt Array |
| 28 | 59 | Male | Large Cell LC | T1aN0M0 | Ia | ✓ | | HuProt Array |
| 29 | 51 | Male | Large Cell LC | T1bN2M0 | IIIa | ✓ | | HuProt Array |
| 30 | 61 | Male | Large Cell LC | T3N2M0 | IIIb | ✓ | | HuProt Array |
| 31 | 53 | Male | Large Cell LC | T2bN2M1a | IV | | | HuProt Array |
| 32 | 52 | Male | Large Cell LC | T3N3M1b | IV | ✓ | | HuProt Array |
| 33 | 52 | Male | Large Cell LC | T2bN3M0 | IIIb | ✓ | | HuProt Array |
| 34 | 54 | Male | Small Cell LC | | Limited | ✓ | | HuProt Array |
| 35 | 55 | Male | Small Cell LC | | Extensive | ✓ | | HuProt Array |
| 36 | 55 | Male | Small Cell LC | | Limited | ✓ | | HuProt Array |
| 37 | 54 | Male | Small Cell LC | | Limited | ✓ | | HuProt Array |
| 38 | 74 | Male | Small Cell LC | | Limited | ✓ | ✓ | HuProt Array |
| 39 | 67 | Male | Small Cell LC | | Limited | ✓ | | HuProt Array |
| 40 | 66 | Male | Small Cell LC | | Extensive | ✓ | | HuProt Array |
| 41 | 69 | Male | Small Cell LC | | Extensive | ✓ | | HuProt Array |
| 42 | 60 | Male | Small Cell LC | | Extensive | ✓ | | HuProt Array |
| 43 | 58 | Male | Small Cell LC | | Extensive | ✓ | | HuProt Array |
| 44 | 63 | Male | Small Cell LC | | Extensive | ✓ | ✓ | HuProt Array |
| 45 | 52 | Male | Small Cell LC | | Limited | ✓ | | HuProt Array |
| 46 | 62 | Male | Small Cell LC | | Extensive | ✓ | | HuProt Array |
| 47 | 47 | Male | Small Cell LC | | Ensive | ✓ | | HuProt Array |
| 48 | 42 | Male | Small Cell LC | | Extensive | ✓ | ✓ | HuProt Array |
| 49 | 66 | Male | Small Cell LC | | Extensive | ✓ | ✓ | HuProt Array |
| 50 | 59 | Male | Small Cell LC | | Limited | ✓ | | HuProt Array |
| 51 | 54 | Male | Small Cell LC | | Extensive | ✓ | ✓ | HuProt Array |
| 52 | 59 | Male | Small Cell LC | | Extensive | ✓ | | HuProt Array |
| 53 | 55 | Male | Small Cell LC | | Extensive | ✓ | | HuProt Array |
| 54 | 63 | Male | Lung Adenocarcinoma | T2aN2M0 | IIIa | | | HuProt Array |
| 55 | 61 | Male | Lung Adenocarcinoma | T1bN2M0 | IIIa | ✓ | | HuProt Array |
| 56 | 69 | Female | Lung Adenocarcinoma | T1cN2M0 | IIIa | | | HuProt Array |
| 57 | 61 | Female | Lung Adenocarcinoma | T4N2M0 | IIIb | | | HuProt Array |
| 58 | 47 | Male | Lung Adenocarcinoma | T2aN3M0 | IIIb | ✓ | ✓ | HuProt Array |
| 59 | 70 | Male | Lung Adenocarcinoma | T4N3M1b | IV | ✓ | ✓ | HuProt Array |
| 60 | 60 | Female | Lung Adenocarcinoma | T3N2M1b | IV | | | HuProt Array |
| 61 | 49 | Male | Lung Adenocarcinoma | T4N3M1b | IV | ✓ | | HuProt Array |
| 62 | 68 | Female | Lung Adenocarcinoma | T2N3M1c | IV | ✓ | | HuProt Array |
| 63 | 51 | Female | Lung Adenocarcinoma | T3N2M1c | IV | | | HuProt Array |
| 64 | 50 | Male | Lung Adenocarcinoma | T4N2M1c | IV | ✓ | ✓ | HuProt Array |
| 65 | 50 | Male | Lung Adenocarcinoma | T4N2M1a | IV | ✓ | | HuProt Array |
| 66 | 77 | Female | Lung Adenocarcinoma | T3N3M1b | IV | | | HuProt Array |
| 67 | 52 | Female | Lung Adenocarcinoma | T4N3M1c | IV | | | HuProt Array |
| 68 | 77 | Male | Lung Adenocarcinoma | T2bN2M1a | IV | ✓ | | HuProt Array |
| 69 | 70 | Male | Lung Adenocarcinoma | T2aN3M1b | IV | | | HuProt Array |
| 70 | 65 | Male | Lung Adenocarcinoma | T2bN2M1b | IV | ✓ | ✓ | HuProt Array |
| 71 | 52 | Female | Lung Adenocarcinoma | T4N3M1b | IV | | | HuProt Array |
| 72 | 77 | Male | Lung Adenocarcinoma | T4N3M1c | IV | ✓ | | HuProt Array |
| 73 | 70 | Female | Lung Adenocarcinoma | T3N2M1c | IV | | | HuProt Array |
| 74 | 58 | Male | Lung Adenocarcinoma | T2bN3M1b | IV | | | HuProt Array |
| 75 | 50 | Male | Lung Adenocarcinoma | T4N2M1b | IV | ✓ | ✓ | HuProt Array |
| 76 | 73 | Female | Lung Adenocarcinoma | T3N3M1b | IV | | | HuProt Array |
| 77 | 64 | Male | Lung Adenocarcinoma | T4N2M1c | IV | ✓ | | HuProt Array |
| 78 | 69 | Male | Squamous Cell LC | T2N3M0 | IIIb | | | HuProt Array |
| 79 | 72 | Male | Squamous Cell LC | T4N2M0 | IIIb | ✓ | | HuProt Array |
| 80 | 64 | Male | Squamous Cell LC | T3N2M0 | IIIb | | | HuProt Array |
| 81 | 50 | Male | Squamous Cell LC | T2aN3M0 | IIIa | ✓ | ✓ | HuProt Array |
| 82 | 62 | Male | Squamous Cell LC | T4N2M1b | IV | ✓ | | HuProt Array |
| 83 | 61 | Male | Squamous Cell LC | T3N3M1b | IV | ✓ | | HuProt Array |
| 84 | 68 | Female | Squamous Cell LC | T3N3M1C | IV | ✓ | ✓ | HuProt Array |
| 85 | 54 | Male | Squamous Cell LC | T4N2M1c | IV | ✓ | ✓ | HuProt Array |
| 86 | 57 | Male | Squamous Cell LC | T3N2M1c | IV | ✓ | | HuProt Array |
| 87 | 54 | Male | Squamous Cell LC | T4N2M1b | IV | | | HuProt Array |
| 88 | 62 | Male | Squamous Cell LC | T4N3M1b | IV | | | HuProt Array |
| 89 | 74 | Male | Squamous Cell LC | T3N2M1c | IV | ✓ | | HuProt Array |
| 90 | 62 | Male | Squamous Cell LC | T4N2M1b | IV | | | HuProt Array |
| 91 | 61 | Male | Squamous Cell LC | T4N3M1b | IV | ✓ | | HuProt Array |
| 92 | 66 | Male | Squamous Cell LC | T3N2M1c | IV | ✓ | ✓ | HuProt Array |
| 93 | 59 | Male | Squamous Cell LC | T2bN3M1c | IV | ✓ | | HuProt Array |
| 94 | 62 | Male | Squamous Cell LC | T3N2M1b | IV | ✓ | ✓ | HuProt Array |
| 95 | 67 | Male | Squamous Cell LC | T4N2M1c | IV | ✓ | | HuProt Array |
| 96 | 69 | Male | Squamous Cell LC | T4N3M1b | IV | | | HuProt Array |
| 97 | 61 | Male | Squamous Cell LC | T4N3M1a | IV | ✓ | | HuProt Array |
| 98 | 54 | Male | Squamous Cell LC | T2bN3M1b | IV | ✓ | | HuProt Array |
| 99 | 49 | Male | Squamous Cell LC | T3N3M1b | IV | ✓ | ✓ | HuProt Array |
| 100 | 66 | Male | Squamous Cell LC | T3N3M1c | IV | ✓ | | HuProt Array |
| 101 | 45 | Male | Healthy | | | ✓ | ✓ | LC Focused Array; ELISA |
| 102 | 46 | Female | Healthy | | | | | LC Focused Array |
| 103 | 46 | Male | Healthy | | | ✓ | ✓ | LC Focused Array; ELISA |
| 104 | 46 | Female | Healthy | | | ✓ | | LC Focused Array |
| 105 | 46 | Female | Healthy | | | | | LC Focused Array; ELISA |
| 106 | 47 | Male | Healthy | | | ✓ | ✓ | LC Focused Array |
| 107 | 47 | Male | Healthy | | | ✓ | ✓ | LC Focused Array; ELISA |
| 108 | 47 | Male | Healthy | | | ✓ | | LC Focused Array; ELISA |
| 109 | 48 | Male | Healthy | | | ✓ | ✓ | LC Focused Array |
| 110 | 48 | Female | Healthy | | | | | LC Focused Array; ELISA |
| 111 | 48 | Female | Healthy | | | | | LC Focused Array |
| 112 | 48 | Male | Healthy | | | | | LC Focused Array |
| 113 | 49 | Female | Healthy | | | ✓ | | LC Focused Array; ELISA |
| 114 | 49 | Male | Healthy | | | ✓ | ✓ | LC Focused Array |
| 115 | 49 | Male | Healthy | | | ✓ | | LC Focused Array; ELISA |
| 116 | 49 | Male | Healthy | | | ✓ | ✓ | LC Focused Array; ELISA |
| 117 | 50 | Male | Healthy | | | ✓ | ✓ | LC Focused Array |
| 118 | 50 | Female | Healthy | | | | | LC Focused Array; ELISA |
| 119 | 51 | Male | Healthy | | | ✓ | | LC Focused Array; ELISA |
| 120 | 51 | Male | Healthy | | | ✓ | ✓ | LC Focused Array; ELISA |
| 121 | 51 | Male | Healthy | | | ✓ | ✓ | LC Focused Array |
| 122 | 51 | Female | Healthy | | | ✓ | | LC Focused Array |
| 123 | 51 | Male | Healthy | | | ✓ | ✓ | LC Focused Array |
| 124 | 52 | Female | Healthy | | | | | LC Focused Array; ELISA |
| 125 | 52 | Male | Healthy | | | ✓ | ✓ | LC Focused Array; ELISA |
| 126 | 52 | Male | Healthy | | | ✓ | | LC Focused Array |
| 127 | 53 | Male | Healthy | | | ✓ | | LC Focused Array; ELISA |
| 128 | 53 | Male | Healthy | | | ✓ | | LC Focused Array; ELISA |
| 129 | 53 | Male | Healthy | | | ✓ | ✓ | LC Focused Array |
| 130 | 53 | Male | Healthy | | | ✓ | ✓ | LC Focused Array; ELISA |
| 131 | 53 | Male | Healthy | | | ✓ | | LC Focused Array; ELISA |
| 132 | 54 | Male | Healthy | | | ✓ | | LC Focused Array |
| 133 | 54 | Male | Healthy | | | ✓ | ✓ | LC Focused Array; ELISA |
| 134 | 54 | Male | Healthy | | | ✓ | ✓ | LC Focused Array; ELISA |
| 135 | 54 | Female | Healthy | | | | | LC Focused Array; ELISA |
| 136 | 54 | Male | Healthy | | | ✓ | | LC Focused Array |
| 137 | 55 | Female | Healthy | | | | | LC Focused Array |
| 138 | 55 | Male | Healthy | | | ✓ | ✓ | LC Focused Array; ELISA |
| 139 | 56 | Male | Healthy | | | ✓ | | LC Focused Array |
| 140 | 56 | Male | Healthy | | | ✓ | | LC Focused Array; ELISA |
| 141 | 56 | Male | Healthy | | | ✓ | ✓ | LC Focused Array; ELISA |
| 142 | 56 | Female | Healthy | | | | | LC Focused Array; ELISA |
| 143 | 56 | Female | Healthy | | | | | LC Focused Array |
| 144 | 56 | Male | Healthy | | | ✓ | | LC Focused Array |
| 145 | 57 | Male | Healthy | | | ✓ | ✓ | LC Focused Array |
| 146 | 57 | Female | Healthy | | | ✓ | | LC Focused Array; ELISA |
| 147 | 57 | Female | Healthy | | | | | LC Focused Array; ELISA |
| 148 | 57 | Male | Healthy | | | ✓ | ✓ | LC Focused Array |
| 149 | 58 | Male | Healthy | | | ✓ | ✓ | LC Focused Array |
| 150 | 58 | Male | Healthy | | | ✓ | | LC Focused Array; ELISA |
| 151 | 58 | Male | Healthy | | | ✓ | | LC Focused Array |
| 152 | 58 | Male | Healthy | | | ✓ | | LC Focused Array |
| 153 | 58 | Female | Healthy | | | | | LC Focused Array |
| 154 | 58 | Female | Healthy | | | | | LC Focused Array |
| 155 | 58 | Female | Healthy | | | | | LC Focused Array; ELISA |
| 156 | 59 | Female | Healthy | | | | | LC Focused Array |
| 157 | 59 | Male | Healthy | | | ✓ | ✓ | LC Focused Array |
| 158 | 59 | Female | Healthy | | | | | LC Focused Array; ELISA |
| 159 | 59 | Male | Healthy | | | ✓ | | LC Focused Array; ELISA |
| 160 | 60 | Male | Healthy | | | ✓ | | LC Focused Array |
| 161 | 61 | Male | Healthy | | | ✓ | ✓ | LC Focused Array |
| 162 | 62 | Male | Healthy | | | ✓ | | LC Focused Array |
| 163 | 62 | Male | Healthy | | | ✓ | | LC Focused Array |
| 164 | 63 | Female | Healthy | | | ✓ | ✓ | LC Focused Array; ELISA |
| 165 | 63 | Male | Healthy | | | ✓ | | LC Focused Array; ELISA |
| 166 | 63 | Male | Healthy | | | ✓ | | LC Focused Array; ELISA |
| 167 | 63 | Male | Healthy | | | ✓ | | LC Focused Array |
| 168 | 64 | Male | Healthy | | | ✓ | | LC Focused Array |
| 169 | 64 | Female | Healthy | | | | | LC Focused Array |
| 170 | 64 | Male | Healthy | | | ✓ | | LC Focused Array; ELISA |
| 171 | 65 | Male | Healthy | | | ✓ | | LC Focused Array |
| 172 | 65 | Male | Healthy | | | ✓ | | LC Focused Array; ELISA |
| 173 | 65 | Male | Healthy | | | ✓ | | LC Focused Array |
| 174 | 66 | Male | Healthy | | | ✓ | | LC Focused Array |
| 175 | 68 | Female | Healthy | | | | | LC Focused Array |
| 176 | 68 | Female | Healthy | | | ✓ | | LC Focused Array |
| 177 | 69 | Male | Healthy | | | ✓ | | LC Focused Array; ELISA |
| 178 | 70 | Male | Healthy | | | ✓ | | LC Focused Array |
| 179 | 70 | Male | Healthy | | | ✓ | | LC Focused Array; ELISA |
| 180 | 70 | Male | Healthy | | | ✓ | | LC Focused Array; ELISA |
| 181 | 70 | Male | Healthy | | | ✓ | | LC Focused Array; ELISA |
| 182 | 70 | Male | Healthy | | | ✓ | | LC Focused Array; ELISA |
| 183 | 71 | Female | Healthy | | | | | LC Focused Array; ELISA |
| 184 | 71 | Male | Healthy | | | ✓ | | LC Focused Array |
| 185 | 71 | Male | Healthy | | | ✓ | | LC Focused Array; ELISA |
| 186 | 72 | Male | Healthy | | | ✓ | | LC Focused Array; ELISA |
| 187 | 72 | Female | Healthy | | | | | LC Focused Array; ELISA |
| 188 | 72 | Male | Healthy | | | ✓ | | LC Focused Array |
| 189 | 72 | Female | Healthy | | | | | LC Focused Array; ELISA |
| 190 | 73 | Female | Healthy | | | | | LC Focused Array |
| 191 | 74 | Female | Healthy | | | ✓ | | LC Focused Array |
| 192 | 75 | Male | Healthy | | | ✓ | | LC Focused Array; ELISA |
| 193 | 76 | Male | Healthy | | | ✓ | | LC Focused Array; ELISA |
| 194 | 40 | Female | Pneumonia | | | ✓ | ✓ | LC Focused Array; ELISA |
| 195 | 44 | Male | Pneumonia | | | ✓ | | LC Focused Array; ELISA |
| 196 | 45 | Male | Pulmonary tuberculosis | | | ✓ | ✓ | LC Focused Array |
| 197 | 46 | Male | Pulmonary tuberculosis | | | | | LC Focused Array |
| 198 | 47 | Female | Pulmonary tuberculosis | | | | | LC Focused Array; ELISA |
| 199 | 47 | Female | Pneumonia | | | | | LC Focused Array |
| 200 | 47 | Male | Pneumonia | | | ✓ | ✓ | LC Focused Array; ELISA |
| 201 | 47 | Male | Chronic obstructive | | | ✓ | | LC Focused Array; ELISA |
| | | | pulmonary disease | | | | | |
| 202 | 48 | Female | Pneumonia | | | | | LC Focused Array; ELISA |
| 203 | 48 | Male | Pulmonary tuberculosis | | | ✓ | ✓ | LC Focused Array; ELISA |
| 204 | 48 | Female | COPD | | | ✓ | ✓ | LC Focused Array |
| 205 | 49 | Female | Pneumonia | | | ✓ | | LC Focused Array |
| 206 | 50 | Male | Pulmonary tuberculosis | | | | | LC Focused Array; ELISA |
| 207 | 50 | Male | Pneumonia | | | ✓ | ✓ | LC Focused Array; ELISA |
| 208 | 50 | Male | Pneumonia | | | ✓ | ✓ | LC Focused Array; ELISA |
| 209 | 50 | Female | Pulmonary tuberculosis | | | | | LC Focused Array; ELISA |
| 210 | 50 | Female | COPD | | | ✓ | | LC Focused Array; ELISA |
| 211 | 51 | Male | Pneumonia | | | ✓ | ✓ | LC Focused Array |
| 212 | 51 | Female | Pneumonia | | | | | LC Focused Array |
| 213 | 51 | Male | Pneumonia | | | ✓ | | LC Focused Array |
| 214 | 51 | Male | Pneumonia | | | ✓ | ✓ | LC Focused Array; ELISA |
| 215 | 52 | Female | Pulmonary tuberculosis | | | | | LC Focused Array |
| 216 | 52 | Female | COPD | | | | | LC Focused Array |
| 217 | 53 | Female | COPD | | | | | LC Focused Array |
| 218 | 53 | Male | Pneumonia | | | ✓ | ✓ | LC Focused Array |
| 219 | 54 | Male | COPD | | | ✓ | | LC Focused Array |
| 220 | 54 | Male | Pneumonia | | | ✓ | | LC Focused Array |
| 221 | 56 | Female | Pneumonia | | | ✓ | ✓ | LC Focused Array |
| 222 | 57 | Male | COPD | | | ✓ | | LC Focused Array |
| 223 | 57 | Female | COPD | | | | | LC Focused Array; ELISA |
| 224 | 57 | Male | COPD | | | ✓ | ✓ | LC Focused Array |
| 225 | 57 | Male | Pulmonary tuberculosis | | | ✓ | ✓ | LC Focused Array; ELISA |
| 226 | 57 | Male | Pneumonia | | | ✓ | | LC Focused Array |
| 227 | 58 | Male | Pneumonia | | | ✓ | | LC Focused Array |
| 228 | 58 | Male | Pulmonary tuberculosis | | | ✓ | ✓ | LC Focused Array |
| 229 | 58 | Male | Pneumonia | | | ✓ | ✓ | LC Focused Array |
| 230 | 59 | Male | Pneumonia | | | ✓ | | LC Focused Array; ELISA |
| 231 | 59 | Female | Pulmonary tuberculosis | | | | | LC Focused Array; ELISA |
| 232 | 59 | Male | COPD | | | ✓ | | LC Focused Array |
| 233 | 60 | Male | Pneumonia | | | ✓ | ✓ | LC Focused Array |
| 234 | 60 | Female | Pneumonia | | | | | LC Focused Array |
| 235 | 60 | Male | Pulmonary tuberculosis | | | | | LC Focused Array; ELISA |
| 236 | 60 | Female | Pneumonia | | | | | LC Focused Array; ELISA |
| 237 | 61 | Male | Pulmonary tuberculosis | | | ✓ | | LC Focused Array; ELISA |
| 238 | 61 | Female | COPD | | | | | LC Focused Array; ELISA |
| 239 | 61 | Male | Pneumonia | | | ✓ | | LC Focused Array; ELISA |
| 240 | 61 | Male | Pneumonia | | | ✓ | | LC Focused Array; ELISA |
| 241 | 61 | Male | Pneumonia | | | ✓ | ✓ | LC Focused Array; ELISA |
| 242 | 61 | Female | COPD | | | | | LC Focused Array; ELISA |
| 243 | 61 | Male | COPD | | | ✓ | | LC Focused Array; ELISA |
| 244 | 61 | Male | Pulmonary tuberculosis | | | | | LC Focused Array |
| 245 | 62 | Female | Pneumonia | | | ✓ | | LC Focused Array |
| 246 | 62 | Male | COPD | | | ✓ | | LC Focused Array |
| 247 | 62 | Male | Pneumonia | | | ✓ | | LC Focused Array |
| 248 | 62 | Male | Pneumonia | | | ✓ | ✓ | LC Focused Array |
| 249 | 63 | Female | COPD | | | ✓ | ✓ | LC Focused Array; ELISA |
| 250 | 63 | Female | Pneumonia | | | | | LC Focused Array; ELISA |
| 251 | 64 | Male | COPD | | | ✓ | | LC Focused Array; ELISA |
| 252 | 64 | Male | COPD | | | ✓ | | LC Focused Array |
| 253 | 64 | Male | Pneumonia | | | ✓ | | LC Focused Array |
| 254 | 64 | Female | Pneumonia | | | | | LC Focused Array |
| 255 | 64 | Male | Pneumonia | | | ✓ | | LC Focused Array |
| 256 | 64 | Male | Pneumonia | | | ✓ | | LC Focused Array |
| 257 | 64 | Male | Pneumonia | | | ✓ | ✓ | LC Focused Array |
| 258 | 65 | Female | COPD | | | ✓ | | LC Focused Array; ELISA |
| 259 | 66 | Male | Pulmonary tuberculosis | | | ✓ | ✓ | LC Focused Array; ELISA |
| 260 | 66 | Male | Pneumonia | | | ✓ | | LC Focused Array; ELISA |
| 261 | 66 | Male | Pneumonia | | | ✓ | | LC Focused Array |
| 262 | 66 | Male | Pulmonary tuberculosis | | | | | LC Focused Array; ELISA |
| 263 | 67 | Female | Pulmonary tuberculosis | | | | | LC Focused Array |
| 264 | 67 | Female | COPD | | | ✓ | | LC Focused Array; ELISA |
| 265 | 67 | Male | Pneumonia | | | ✓ | | LC Focused Array; ELISA |
| 266 | 67 | Male | Pneumonia | | | ✓ | | LC Focused Array |
| 267 | 67 | Male | Pneumonia | | | ✓ | | LC Focused Array |
| 268 | 68 | Female | Pneumonia | | | | | LC Focused Array; ELISA |
| 269 | 68 | Male | COPD | | | ✓ | | LC Focused Array |
| 270 | 69 | Male | COPD | | | ✓ | | LC Focused Array; ELISA |
| 271 | 69 | Male | Pulmonary tuberculosis | | | | | LC Focused Array; ELISA |
| 272 | 69 | Male | Pneumonia | | | ✓ | | LC Focused Array |
| 273 | 69 | Male | Pneumonia | | | ✓ | | LC Focused Array |
| 274 | 70 | Male | Pneumonia | | | ✓ | | LC Focused Array; ELISA |
| 275 | 70 | Male | Pneumonia | | | ✓ | | LC Focused Array; ELISA |
| 276 | 70 | Male | Pulmonary tuberculosis | | | ✓ | | LC Focused Array; ELISA |
| 277 | 70 | Female | Pulmonary tuberculosis | | | | | LC Focused Array; ELISA |
| 278 | 70 | Female | Pneumonia | | | ✓ | | LC Focused Array |
| 279 | 70 | Male | Pneumonia | | | ✓ | | LC Focused Array |
| 280 | 71 | Male | COPD | | | ✓ | | LC Focused Array; ELISA |
| 281 | 71 | Male | Pneumonia | | | ✓ | | LC Focused Array; ELISA |
| 282 | 72 | Male | Pneumonia | | | ✓ | | LC Focused Array; ELISA |
| 283 | 72 | Male | Pneumonia | | | ✓ | | LC Focused Array; ELISA |
| 284 | 72 | Female | COPD | | | ✓ | | LC Focused Array |
| 285 | 72 | Male | Pulmonary tuberculosis | | | ✓ | | LC Focused Array |
| 286 | 72 | Male | Pneumonia | | | ✓ | | LC Focused Array |
| 287 | 73 | Male | Pneumonia | | | ✓ | | LC Focused Array |
| 288 | 73 | Male | COPD | | | ✓ | | LC Focused Array |
| 289 | 74 | Female | COPD | | | | | LC Focused Array; ELISA |
| 290 | 74 | Male | COPD | | | ✓ | | LC Focused Array |
| 291 | 75 | Male | Pneumonia | | | | | LC Focused Array; ELISA |
| 292 | 76 | Male | Pneumonia | | | ✓ | | LC Focused Array; ELISA |
| 293 | 77 | Male | Pneumonia | | | ✓ | | LC Focused Array; ELISA |
| 294 | 78 | Female | Pneumonia | | | | | LC Focused Array |
| 295 | 65 | Male | Small Cell LC | | Extensive | ✓ | | LC Focused Array; ELISA |
| 296 | 76 | Male | Small Cell LC | | Extensive | ✓ | | LC Focused Array; ELISA |
| 297 | 56 | Male | Small Cell LC | | Extensive | ✓ | | LC Focused Array; ELISA |
| 298 | 51 | Male | Small Cell LC | | Extensive | ✓ | ✓ | LC Focused Array; ELISA |
| 299 | 68 | Male | Small Cell LC | | Extensive | ✓ | | LC Focused Array; ELISA |
| 300 | 57 | Male | Small Cell LC | | Extensive | ✓ | | LC Focused Array; ELISA |
| 301 | 67 | Male | Small Cell LC | | Extensive | ✓ | | LC Focused Array; ELISA |
| 302 | 67 | Male | Small Cell LC | | Extensive | ✓ | | LC Focused Array; ELISA |
| 303 | 67 | Male | Small Cell LC | | Extensive | ✓ | | LC Focused Array; ELISA |
| 304 | 56 | Male | Small Cell LC | | Extensive | ✓ | ✓ | LC Focused Array; ELISA |
| 305 | 65 | Male | Small Cell LC | | Extensive | ✓ | | LC Focused Array; ELISA |
| 306 | 51 | Male | Small Cell LC | | Extensive | ✓ | ✓ | LC Focused Array; ELISA |
| 307 | 68 | Male | Small Cell LC | | Extensive | ✓ | | LC Focused Array; ELISA |
| 308 | 65 | Male | Small Cell LC | | Extensive | ✓ | | LC Focused Array; ELISA |
| 309 | 65 | Male | Small Cell LC | | Extensive | ✓ | | LC Focused Array |
| 310 | 69 | Male | Small Cell LC | | Extensive | ✓ | | LC Focused Array |
| 311 | 56 | Male | Small Cell LC | | Extensive | ✓ | ✓ | LC Focused Array |
| 312 | 63 | Male | Small Cell LC | | Extensive | ✓ | | LC Focused Array |
| 313 | 54 | Male | Small Cell LC | | Extensive | ✓ | ✓ | LC Focused Array |
| 314 | 62 | Male | Small Cell LC | | Extensive | ✓ | | LC Focused Array |
| 315 | 56 | Male | Small Cell LC | | Extensive | ✓ | ✓ | LC Focused Array |
| 316 | 55 | Male | Small Cell LC | | Extensive | ✓ | | LC Focused Array |
| 317 | 67 | Male | Small Cell LC | | Extensive | ✓ | | LC Focused Array |
| 318 | 55 | Male | Small Cell LC | | Extensive | ✓ | ✓ | LC Focused Array |
| 319 | 63 | Male | Small Cell LC | | Extensive | ✓ | | LC Focused Array |
| 320 | 69 | Male | Small Cell LC | | Extensive | ✓ | | LC Focused Array |
| 321 | 56 | Male | Small Cell LC | | Extensive | ✓ | ✓ | LC Focused Array |
| 322 | 61 | Male | Small Cell LC | | Extensive | ✓ | | LC Focused Array |
| 323 | 62 | Male | Small Cell LC | | Extensive | ✓ | | LC Focused Array |
| 324 | 60 | Male | Small Cell LC | | Extensive | ✓ | | LC Focused Array; ELISA |
| 325 | 61 | Male | Small Cell LC | | Extensive | ✓ | | LC Focused Array; ELISA |
| 326 | 64 | Male | Small Cell LC | | Extensive | ✓ | | LC Focused Array; ELISA |
| 327 | 64 | Male | Small Cell LC | | Extensive | ✓ | | LC Focused Array; ELISA |
| 328 | 64 | Male | Small Cell LC | | Extensive | ✓ | | LC Focused Array; ELISA |
| 329 | 62 | Male | Small Cell LC | | Extensive | ✓ | ✓ | LC Focused Array; ELISA |
| 330 | 62 | Male | Small Cell LC | | Extensive | ✓ | | LC Focused Array; ELISA |
| 331 | 61 | Male | Small Cell LC | | Extensive | ✓ | | LC Focused Array |
| 332 | 65 | Male | Small Cell LC | | Extensive | ✓ | | LC Focused Array |
| 333 | 69 | Male | Small Cell LC | | Extensive | ✓ | | LC Focused Array |
| 334 | 59 | Male | Small Cell LC | | Extensive | ✓ | | LC Focused Array |
| 335 | 51 | Male | Small Cell LC | | Extensive | ✓ | ✓ | LC Focused Array |
| 336 | 68 | Male | Small Cell LC | | Extensive | ✓ | | LC Focused Array |
| 337 | 64 | Male | Small Cell LC | | Extensive | ✓ | | LC Focused Array |
| 338 | 76 | Male | Small Cell LC | | Limited | ✓ | | LC Focused Array; ELISA |
| 339 | 66 | Male | Small Cell LC | | Limited | ✓ | | LC Focused Array; ELISA |
| 340 | 66 | Male | Small Cell LC | | Limited | ✓ | | LC Focused Array; ELISA |
| 341 | 51 | Male | Small Cell LC | | Limited | ✓ | ✓ | LC Focused Array; ELISA |
| 342 | 62 | Male | Small Cell LC | | Limited | ✓ | | LC Focused Array; ELISA |
| 343 | 52 | Male | Small Cell LC | | Limited | ✓ | ✓ | LC Focused Array; ELISA |
| 344 | 51 | Male | Small Cell LC | | Limited | ✓ | | LC Focused Array; ELISA |
| 345 | 56 | Male | Small Cell LC | | Limited | ✓ | | LC Focused Array; ELISA |
| 346 | 60 | Male | Small Cell LC | | Limited | ✓ | | LC Focused Array; ELISA |
| 347 | 62 | Male | Small Cell LC | | Limited | ✓ | | LC Focused Array; ELISA |
| 348 | 64 | Female | Small Cell LC | | Limited | ✓ | | LC Focused Array; ELISA |
| 349 | 63 | Male | Small Cell LC | | Limited | ✓ | | LC Focused Array; ELISA |
| 350 | 61 | Male | Small Cell LC | | Limited | ✓ | | LC Focused Array |
| 351 | 75 | Male | Small Cell LC | | Limited | ✓ | | LC Focused Array |
| 352 | 74 | Male | Small Cell LC | | Limited | ✓ | | LC Focused Array |
| 353 | 61 | Male | Small Cell LC | | Limited | ✓ | ✓ | LC Focused Array |
| 354 | 70 | Male | Small Cell LC | | Limited | ✓ | | LC Focused Array |
| 355 | 68 | Female | Small Cell LC | | Limited | ✓ | | LC Focused Array |
| 356 | 45 | Male | Small Cell LC | | Limited | ✓ | ✓ | LC Focused Array |
| 357 | 70 | Male | Small Cell LC | | Limited | ✓ | | LC Focused Array |
| 358 | 61 | Male | Small Cell LC | | Limited | ✓ | | LC Focused Array |
| 359 | 61 | Male | Small Cell LC | | Limited | ✓ | | LC Focused Array |
| 360 | 60 | Male | Small Cell LC | | Limited | ✓ | | LC Focused Array; ELISA |
| 361 | 50 | Male | Small Cell LC | | Limited | ✓ | | LC Focused Array; ELISA |
| 362 | 21 | Female | Small Cell LC | | Limited | ✓ | ✓ | LC Focused Array; ELISA |
| 363 | 76 | Male | Small Cell LC | | Limited | ✓ | | LC Focused Array; ELISA |
| 364 | 46 | Male | Small Cell LC | | Limited | ✓ | | LC Focused Array; ELISA |
| 365 | 60 | Male | Small Cell LC | | Limited | ✓ | ✓ | LC Focused Array; ELISA |
| 366 | 71 | Male | Small Cell LC | | Limited | ✓ | | LC Focused Array |
| 367 | 63 | Male | Small Cell LC | | Limited | ✓ | | LC Focused Array |
| 368 | 49 | Female | Lung Adenocarcinoma | T1bN0M0 | Ia | | | LC Focused Array |
| 369 | 44 | Female | Lung Adenocarcinoma | T1bN0M0 | Ia | | | LC Focused Array |
| 370 | 68 | Male | Lung Adenocarcinoma | T1aN0M0 | Ia | | | LC Focused Array |
| 371 | 74 | Female | Lung Adenocarcinoma | T1aN0M0 | Ia | ✓ | | LC Focused Array |
| 372 | 52 | Male | Lung Adenocarcinoma | T2aN0M0 | Ib | ✓ | | LC Focused Array |
| 373 | 70 | Female | Lung Adenocarcinoma | T1N0M0 | Ia | ✓ | | LC Focused Array |
| 374 | 45 | Female | Lung Adenocarcinoma | T1aN0M0 | Ia | ✓ | ✓ | LC Focused Array; ELISA |
| 375 | 70 | Female | Lung Adenocarcinoma | T1bN0M0 | Ia | | | LC Focused Array; ELISA |
| 376 | 78 | Female | Lung Adenocarcinoma | T1aN0M0 | Ia | | | LC Focused Array; ELISA |
| 377 | 57 | Male | Lung Adenocarcinoma | T2aN0M0 | Ib | ✓ | | LC Focused Array; ELISA |
| 378 | 52 | Female | Lung Adenocarcinoma | T2aN0M0 | Ib | ✓ | ✓ | LC Focused Array; ELISA |
| 379 | 63 | Male | Lung Adenocarcinoma | T2aN0M0 | Ib | ✓ | | LC Focused Array; ELISA |
| 380 | 67 | Female | Lung Adenocarcinoma | T1aN0M0 | Ia | | | LC Focused Array; ELISA |
| 381 | 51 | Female | Lung Adenocarcinoma | T1bN1M0 | Ia | | | LC Focused Array; ELISA |
| 382 | 51 | Male | Lung Adenocarcinoma | T1aN0M0 | Ia | ✓ | ✓ | LC Focused Array; ELISA |
| 383 | 48 | Female | Lung Adenocarcinoma | T2aN0M0 | Ib | | | LC Focused Array; ELISA |
| 384 | 67 | Male | Lung Adenocarcinoma | T2aN0M0 | Ib | ✓ | | LC Focused Array; ELISA |
| 385 | 40 | Female | Lung Adenocarcinoma | T2aN0M0 | Ib | ✓ | | LC Focused Array; ELISA |
| 386 | 60 | Male | Lung Adenocarcinoma | T1aN0M0 | Ia | ✓ | ✓ | LC Focused Array; ELISA |
| 387 | 65 | Male | Lung Adenocarcinoma | T1bN0M0 | Ia | ✓ | | LC Focused Array; ELISA |
| 388 | 51 | Male | Lung Adenocarcinoma | T1bN0M0 | Ia | ✓ | | LC Focused Array; ELISA |
| 389 | 40 | Female | Lung Adenocarcinoma | T2aN0M0 | Ib | ✓ | ✓ | LC Focused Array |
| 390 | 57 | Male | Lung Adenocarcinoma | T1bN0M0 | Ia | | | LC Focused Array |
| 391 | 77 | Male | Lung Adenocarcinoma | T1aN0M0 | Ia | ✓ | | LC Focused Array |
| 392 | 79 | Male | Lung Adenocarcinoma | T1aN0M0 | Ia | ✓ | | LC Focused Array |
| 393 | 62 | Female | Lung Adenocarcinoma | T2aN0M0 | Ib | | | LC Focused Array |
| 394 | 65 | Male | Lung Adenocarcinoma | T2aN0M0 | Ib | ✓ | | LC Focused Array |
| 395 | 67 | Female | Lung Adenocarcinoma | T2aN0M0 | Ib | | | LC Focused Array |
| 396 | 67 | Male | Lung Adenocarcinoma | T2aN0M0 | Ib | ✓ | | LC Focused Array |
| 397 | 40 | Female | Lung Adenocarcinoma | T2aN0M0 | Ib | ✓ | ✓ | LC Focused Array |
| 398 | 60 | Male | Lung Adenocarcinoma | T2aN0M0 | Ib | ✓ | ✓ | LC Focused Array |
| 399 | 75 | Female | Lung Adenocarcinoma | T1bN0M0 | Ia | | | LC Focused Array |
| 400 | 61 | Female | Lung Adenocarcinoma | T1aN0M0 | Ia | | | LC Focused Array |
| 401 | 59 | Female | Lung Adenocarcinoma | T1bN0M0 | Ia | ✓ | | LC Focused Array |
| 402 | 74 | Male | Lung Adenocarcinoma | T2N0M0 | Ib | ✓ | | LC Focused Array |
| 403 | 55 | Female | Lung Adenocarcinoma | T1aN0M0 | Ia | ✓ | ✓ | LC Focused Array |
| 404 | 71 | Male | Lung Adenocarcinoma | T1aN0M0 | Ia | ✓ | | LC Focused Array |
| 405 | 66 | Male | Lung Adenocarcinoma | T2aN0M0 | Ib | ✓ | | LC Focused Array |
| 406 | 54 | Male | Lung Adenocarcinoma | T2aN0M0 | Ib | ✓ | ✓ | LC Focused Array |
| 407 | 57 | Female | Lung Adenocarcinoma | T1aN0M0 | Ia | | | LC Focused Array |
| 408 | 55 | Male | Lung Adenocarcinoma | T2bN0M0 | IIa | ✓ | | LC Focused Array; ELISA |
| 409 | 60 | Male | Lung Adenocarcinoma | T2bN0M0 | IIa | | | LC Focused Array; ELISA |
| 410 | 59 | Male | Lung Adenocarcinoma | T2aN1MO | IIb | | | LC Focused Array; ELISA |
| 411 | 67 | Female | Lung Adenocarcinoma | T3N0M0 | IIb | | | LC Focused Array |
| 412 | 65 | Male | Lung Adenocarcinoma | T2aN1M0 | IIb | ✓ | | LC Focused Array; ELISA |
| 413 | 45 | Female | Lung Adenocarcinoma | T1aN1M0 | IIb | | | LC Focused Array; ELISA |
| 414 | 73 | Male | Lung Adenocarcinoma | T2bN0M0 | IIa | ✓ | | LC Focused Array; ELISA |
| 415 | 58 | Male | Lung Adenocarcinoma | T2bN0M0 | IIa | ✓ | | LC Focused Array; ELISA |
| 416 | 52 | Male | Lung Adenocarcinoma | T2aN1M0 | IIb | ✓ | ✓ | LC Focused Array; ELISA |
| 417 | 67 | Male | Lung Adenocarcinoma | T2bN1MO | IIb | ✓ | | LC Focused Array; ELISA |
| 418 | 75 | Male | Lung Adenocarcinoma | T3N0M0 | IIb | ✓ | | LC Focused Array |
| 419 | 57 | Female | Lung Adenocarcinoma | T2bN0M0 | IIa | ✓ | | LC Focused Array |
| 420 | 52 | Male | Lung Adenocarcinoma | T2bN0M0 | IIa | ✓ | ✓ | LC Focused Array |
| 421 | 55 | Male | Lung Adenocarcinoma | T3NOMO | IIb | ✓ | | LC Focused Array |
| 422 | 48 | Male | Lung Adenocarcinoma | T2bN1M0 | IIb | ✓ | ✓ | LC Focused Array |
| 423 | 58 | Female | Lung Adenocarcinoma | T2aN2M0 | IIIa | | | LC Focused Array |
| 424 | 58 | Female | Lung Adenocarcinoma | T2aN2M0 | IIIa | | | LC Focused Array |
| 425 | 64 | Female | Lung Adenocarcinoma | T1bN2M0 | IIIa | | | LC Focused Array |
| 426 | 63 | Male | Lung Adenocarcinoma | T1aN2M0 | IIIa | ✓ | ✓ | LC Focused Array |
| 427 | 71 | Male | Lung Adenocarcinoma | T4N2M0 | IIIb | ✓ | | LC Focused Array |
| 428 | 70 | Male | Lung Adenocarcinoma | T2bN2M0 | IIIb | ✓ | | LC Focused Array |
| 429 | 65 | Male | Lung Adenocarcinoma | T3N1M0 | IIIa | ✓ | | LC Focused Array |
| 430 | 52 | Male | Lung Adenocarcinoma | T2bN2M0 | IIIa | ✓ | ✓ | LC Focused Array |
| 431 | 51 | Female | Lung Adenocarcinoma | T4N1M0 | IIIa | | | LC Focused Array |
| 432 | 59 | Female | Lung Adenocarcinoma | T2aN2M0 | IIIa | | | LC Focused Array; ELISA |
| 433 | 65 | Female | Lung Adenocarcinoma | T2bN2M0 | IIIa | | | LC Focused Array; ELISA |
| 434 | 65 | Female | Lung Adenocarcinoma | T3N2M0 | IIIb | ✓ | | LC Focused Array; ELISA |
| 435 | 54 | Female | Lung Adenocarcinoma | T2bN3M0 | IIIb | | | LC Focused Array; ELISA |
| 436 | 58 | Female | Lung Adenocarcinoma | T2aN3M0 | IIIb | | | LC Focused Array; ELISA |
| 437 | 54 | Male | Lung Adenocarcinoma | T3N2M0 | IIIb | ✓ | ✓ | LC Focused Array; ELISA |
| 438 | 44 | Female | Lung Adenocarcinoma | T4N1M0 | IIIa | | | LC Focused Array; ELISA |
| 439 | 72 | Female | Lung Adenocarcinoma | T1aN2M0 | IIIa | | | LC Focused Array; ELISA |
| 440 | 58 | Male | Lung Adenocarcinoma | T1bN2M0 | IIIa | ✓ | | LC Focused Array; ELISA |
| 441 | 36 | Female | Lung Adenocarcinoma | T4N2M0 | IIIb | ✓ | ✓ | LC Focused Array; ELISA |
| 442 | 52 | Male | Lung Adenocarcinoma | T1bN2M0 | IIIb | ✓ | ✓ | LC Focused Array; ELISA |
| 443 | 65 | Male | Lung Adenocarcinoma | T2aN3M0 | IIIb | | | LC Focused Array |
| 444 | 57 | Female | Lung Adenocarcinoma | T3N1M0 | IIIa | | | LC Focused Array; ELISA |
| 445 | 82 | Male | Lung Adenocarcinoma | T4N1M0 | IIIa | ✓ | | LC Focused Array |
| 446 | 64 | Male | Lung Adenocarcinoma | T1bN2M0 | IIIa | ✓ | | LC Focused Array |
| 447 | 54 | Male | Lung Adenocarcinoma | T1bN2M0 | IIIa | ✓ | ✓ | LC Focused Array |
| 448 | 75 | Male | Lung Adenocarcinoma | T4N2M0 | IIIb | ✓ | | LC Focused Array |
| 449 | 76 | Male | Lung Adenocarcinoma | T2bN3M0 | IIIb | ✓ | | LC Focused Array |
| 450 | 70 | Male | Lung Adenocarcinoma | T1bN3M0 | IIIb | ✓ | | LC Focused Array |
| 451 | 59 | Female | Lung Adenocarcinoma | T3N1M0 | IIIa | | | LC Focused Array |
| 452 | 69 | Male | Lung Adenocarcinoma | T4N1M0 | IIIa | ✓ | | LC Focused Array |
| 453 | 63 | Male | Lung Adenocarcinoma | T4aN3M1b | IV | ✓ | ✓ | LC Focused Array; ELISA |
| 454 | 62 | Female | Lung Adenocarcinoma | T4N2M1b | IV | | | LC Focused Array |
| 455 | 50 | Female | Lung Adenocarcinoma | T4N2M1b | IV | | | LC Focused Array |
| 456 | 61 | Male | Lung Adenocarcinoma | T3N2M1a | IV | ✓ | ✓ | LC Focused Array |
| 457 | 73 | Female | Lung Adenocarcinoma | T4N2M1a | IV | ✓ | | LC Focused Array |
| 458 | 58 | Female | Lung Adenocarcinoma | T4N3M1c | IV | | | LC Focused Array |
| 459 | 49 | Male | Lung Adenocarcinoma | T4N2M1c | IV | | | LC Focused Array |
| 460 | 48 | Female | Lung Adenocarcinoma | T4N2M1c | IV | | | LC Focused Array |
| 461 | 66 | Female | Lung Adenocarcinoma | T4N3M1b | IV | | | LC Focused Array |
| 462 | 60 | Male | Lung Adenocarcinoma | T3N2M1b | IV | ✓ | | LC Focused Array |
| 463 | 77 | Male | Lung Adenocarcinoma | T4N2M1b | IV | ✓ | | LC Focused Array |
| 464 | 64 | Male | Lung Adenocarcinoma | T3N2M1b | IV | ✓ | | LC Focused Array |
| 465 | 42 | Male | Lung Adenocarcinoma | T4N2M1b | IV | ✓ | ✓ | LC Focused Array |
| 466 | 62 | Female | Lung Adenocarcinoma | T4aN3M1a | IV | ✓ | | LC Focused Array |
| 467 | 52 | Male | Lung Adenocarcinoma | T4N2M1c | IV | ✓ | ✓ | LC Focused Array |
| 468 | 52 | Female | Lung Adenocarcinoma | T4N2M1b | IV | | | LC Focused Array |
| 469 | 46 | Female | Lung Adenocarcinoma | T3N3M1a | IV | | | LC Focused Array; ELISA |
| 470 | 55 | Female | Lung Adenocarcinoma | T3N3M1b | IV | | | LC Focused Array; ELISA |
| 471 | 74 | Female | Lung Adenocarcinoma | T4N2M1a | IV | | | LC Focused Array; ELISA |
| 472 | 68 | Male | Lung Adenocarcinoma | T4N2M1c | IV | ✓ | | LC Focused Array; ELISA |
| 473 | 53 | Male | Lung Adenocarcinoma | T4N3M1c | IV | ✓ | | LC Focused Array; ELISA |
| 474 | 72 | Female | Lung Adenocarcinoma | T2aN3M1c | IV | | | LC Focused Array; ELISA |
| 475 | 60 | Male | Lung Adenocarcinoma | T2aN3M1c | IV | ✓ | | LC Focused Array |
| 476 | 43 | Male | Lung Adenocarcinoma | T2bN2M1b | IV | ✓ | ✓ | LC Focused Array |
| 477 | 39 | Male | Lung Adenocarcinoma | T4N3M1b | IV | ✓ | ✓ | LC Focused Array |
| 478 | 75 | Female | Lung Adenocarcinoma | T4N3M1b | IV | | | LC Focused Array |
| 479 | 68 | Male | Lung Adenocarcinoma | T4N2M1b | IV | ✓ | | LC Focused Array |
| 480 | 66 | Male | Lung Adenocarcinoma | T2bN3M1a | IV | ✓ | | LC Focused Array |
| 481 | 46 | Female | Lung Adenocarcinoma | T4N3M1b | IV | | | LC Focused Array |
| 482 | 70 | Male | Lung Adenocarcinoma | T4N2M1c | IV | | | LC Focused Array |
| 483 | 68 | Female | Lung Adenocarcinoma | T4N3M1b | IV | | | LC Focused Array |
| 484 | 58 | Male | Lung Adenocarcinoma | T2N3M1c | IV | ✓ | ✓ | LC Focused Array |
| 485 | 62 | Male | Lung Adenocarcinoma | T3N2M1c | IV | ✓ | | LC Focused Array |
| 486 | 61 | Female | Lung Adenocarcinoma | T4N2M1b | IV | | | LC Focused Array |
| 487 | 60 | Female | Lung Adenocarcinoma | T4N2M1a | IV | | | LC Focused Array |
| 488 | 58 | Male | Lung Adenocarcinoma | T4N2M1b | IV | ✓ | ✓ | LC Focused Array |
| 489 | 64 | Male | Lung Adenocarcinoma | T4N3M1b | IV | ✓ | | LC Focused Array |
| 490 | 75 | Male | Lung Adenocarcinoma | T2bN2M1b | IV | ✓ | | LC Focused Array |
| 491 | 66 | Male | Lung Adenocarcinoma | T2aN3M1c | IV | ✓ | | LC Focused Array |
| 492 | 42 | Female | Lung Adenocarcinoma | T2bN2M1c | IV | | | LC Focused Array |
| 493 | 73 | Male | Lung Adenocarcinoma | T4N3M1a | IV | ✓ | | LC Focused Array |
| 494 | 61 | Female | Lung Adenocarcinoma | T4N3M1b | IV | | | LC Focused Array |
| 495 | 65 | Female | Lung Adenocarcinoma | T4N2M1b | IV | | | LC Focused Array |
| 496 | 65 | Male | Lung Adenocarcinoma | T4N2M1b | IV | ✓ | | LC Focused Array |
| 497 | 64 | Female | Lung Adenocarcinoma | T2bN3M1a | IV | | | LC Focused Array |
| 498 | 62 | Female | Lung Adenocarcinoma | T4N3M1b | IV | | | LC Focused Array |
| 499 | 50 | Male | Lung Adenocarcinoma | T3N3M1b | IV | ✓ | ✓ | LC Focused Array |
| 500 | 61 | Male | Lung Adenocarcinoma | T4N3M1c | IV | ✓ | ✓ | LC Focused Array |
| 501 | 67 | Female | Lung Adenocarcinoma | T2aN3M1c | IV | ✓ | | LC Focused Array |
| 502 | 66 | Male | Lung Adenocarcinoma | T3N2M1b | IV | ✓ | | LC Focused Array |
| 503 | 37 | Male | Lung Adenocarcinoma | T4N2M1b | IV | ✓ | ✓ | LC Focused Array |
| 504 | 65 | Male | Lung Adenocarcinoma | T4N2M1a | IV | ✓ | | LC Focused Array; ELISA |
| 505 | 58 | Male | Lung Adenocarcinoma | T4N2M1b | IV | ✓ | | LC Focused Array |
| 506 | 65 | Female | Lung Adenocarcinoma | T3N3M1c | IV | | | LC Focused Array |
| 507 | 58 | Male | Lung Adenocarcinoma | T3N3M1b | IV | ✓ | | LC Focused Array |
| 508 | 62 | Female | Lung Adenocarcinoma | T4N2M1c | IV | ✓ | | LC Focused Array |
| 509 | 49 | Male | Lung Adenocarcinoma | T3N2M1c | IV | ✓ | ✓ | LC Focused Array |
| 510 | 62 | Female | Lung Adenocarcinoma | T4N2M1b | IV | | | LC Focused Array |
| 511 | 76 | Female | Lung Adenocarcinoma | T4N3M1b | IV | | | LC Focused Array |
| 512 | 47 | Male | Lung Adenocarcinoma | T4N2M1c | IV | | | LC Focused Array |
| 513 | 56 | Female | Lung Adenocarcinoma | T4N2M1c | IV | | | LC Focused Array |
| 514 | 50 | Male | Lung Adenocarcinoma | T4N3M1a | IV | ✓ | ✓ | LC Focused Array |
| 515 | 58 | Male | Lung Adenocarcinoma | T3N2M1c | IV | ✓ | | LC Focused Array |
| 516 | 41 | Female | Lung Adenocarcinoma | T3N2M1b | IV | | | LC Focused Array |
| 517 | 62 | Female | Lung Adenocarcinoma | T4N2M1b | IV | | | LC Focused Array |
| 518 | 52 | Female | Lung Adenocarcinoma | T4N3M1a | IV | | | LC Focused Array |
| 519 | 71 | Female | Lung Adenocarcinoma | T4N2M1b | IV | | | LC Focused Array |
| 520 | 66 | Male | Lung Adenocarcinoma | T4N2M1c | IV | ✓ | ✓ | LC Focused Array |
| 521 | 58 | Male | Lung Adenocarcinoma | T4N3M1c | IV | ✓ | ✓ | LC Focused Array |
| 522 | 59 | Male | Squamous Cell LC | T1aN0M0 | Ia | ✓ | | LC Focused Array; ELISA |
| 523 | 62 | Male | Squamous Cell LC | T1aN0M0 | Ia | ✓ | | LC Focused Array; ELISA |
| 524 | 72 | Male | Squamous Cell LC | T2aN0M0 | Ib | ✓ | | LC Focused Array; ELISA |
| 525 | 57 | Male | Squamous Cell LC | T1cN0M0 | Ia | | | LC Focused Array; ELISA |
| 526 | 76 | Male | Squamous Cell LC | T1aN0M0 | Ia | ✓ | | LC Focused Array; ELISA |
| 527 | 59 | Male | Squamous Cell LC | T1aN0M0 | Ia | ✓ | ✓ | LC Focused Array; ELISA |
| 528 | 68 | Male | Squamous Cell LC | T1aN0M0 | Ia | ✓ | | LC Focused Array; ELISA |
| 529 | 69 | Male | Squamous Cell LC | T2aN0M0 | Ib | | | LC Focused Array; ELISA |
| 530 | 57 | Male | Squamous Cell LC | T2aN0M0 | Ib | ✓ | ✓ | LC Focused Array; ELISA |
| 531 | 65 | Male | Squamous Cell LC | T2aN0M0 | Ib | ✓ | | LC Focused Array; ELISA |
| 532 | 54 | Male | Squamous Cell LC | T1aN0M0 | Ia | ✓ | ✓ | LC Focused Array; ELISA |
| 533 | 58 | Male | Squamous Cell LC | T1bN1M0 | Ia | ✓ | | LC Focused Array; ELISA |
| 534 | 49 | Male | Squamous Cell LC | T1aN0M0 | Ia | ✓ | ✓ | LC Focused Array |
| 535 | 70 | Male | Squamous Cell LC | T2aN0M0 | Ib | ✓ | | LC Focused Array; ELISA |
| 536 | 66 | Male | Squamous Cell LC | T2aN0M0 | Ib | ✓ | | LC Focused Array |
| 537 | 67 | Male | Squamous Cell LC | T2aN0M0 | Ib | ✓ | | LC Focused Array; ELISA |
| 538 | 67 | Male | Squamous Cell LC | T1aN0M0 | Ia | ✓ | | LC Focused Array; ELISA |
| 539 | 57 | Male | Squamous Cell LC | T1aN0M0 | Ia | ✓ | ✓ | LC Focused Array |
| 540 | 46 | Male | Squamous Cell LC | T1bN0M0 | Ia | ✓ | ✓ | LC Focused Array; ELISA |
| 541 | 66 | Male | Squamous Cell LC | T2N0M0 | Ib | ✓ | | LC Focused Array |
| 542 | 64 | Male | Squamous Cell LC | T1aN0M0 | Ia | ✓ | | LC Focused Array |
| 543 | 71 | Male | Squamous Cell LC | T1aN0M0 | Ia | ✓ | | LC Focused Array |
| 544 | 55 | Male | Squamous Cell LC | T1aN0M0 | Ia | ✓ | ✓ | LC Focused Array |
| 545 | 47 | Male | Squamous Cell LC | T2aN0M0 | Ib | ✓ | ✓ | LC Focused Array |
| 546 | 58 | Male | Squamous Cell LC | T2aN0M0 | Ib | | | LC Focused Array |
| 547 | 71 | Female | Squamous Cell LC | T2aN0M0 | Ib | | | LC Focused Array |
| 548 | 81 | Male | Squamous Cell LC | T2aN0M0 | Ib | ✓ | | LC Focused Array |
| 549 | 60 | Male | Squamous Cell LC | T2aN0M0 | Ib | ✓ | | LC Focused Array |
| 550 | 64 | Male | Squamous Cell LC | T1aN0M0 | Ia | ✓ | | LC Focused Array |
| 551 | 56 | Male | Squamous Cell LC | T1cN0M0 | Ia | ✓ | ✓ | LC Focused Array |
| 552 | 74 | Male | Squamous Cell LC | T2bN0M0 | IIa | ✓ | | LC Focused Array; ELISA |
| 553 | 48 | Female | Squamous Cell LC | T3NOMO | IIb | | | LC Focused Array; ELISA |
| 554 | 67 | Male | Squamous Cell LC | T3N0M0 | IIb | ✓ | | LC Focused Array; ELISA |
| 555 | 68 | Male | Squamous Cell LC | T2bN0M0 | IIa | ✓ | | LC Focused Array; ELISA |
| 556 | 65 | Male | Squamous Cell LC | T2bN0M0 | IIa | ✓ | | LC Focused Array; ELISA |
| 557 | 58 | Female | Squamous Cell LC | T2aN1MO | IIb | ✓ | | LC Focused Array; ELISA |
| 558 | 70 | Male | Squamous Cell LC | T3N0M0 | IIb | ✓ | | LC Focused Array; ELISA |
| 559 | 58 | Male | Squamous Cell LC | T2bN0M0 | IIa | ✓ | | LC Focused Array; ELISA |
| 560 | 76 | Male | Squamous Cell LC | T2bN0M0 | IIa | ✓ | | LC Focused Array; ELISA |
| 561 | 63 | Male | Squamous Cell LC | T3NOMO | IIb | ✓ | | LC Focused Array |
| 562 | 59 | Male | Squamous Cell LC | T3N0M0 | IIb | ✓ | ✓ | LC Focused Array |
| 563 | 79 | Male | Squamous Cell LC | T2aN1M0 | IIb | ✓ | | LC Focused Array |
| 564 | 51 | Male | Squamous Cell LC | T2bN0M0 | IIa | | | LC Focused Array |
| 565 | 69 | Male | Squamous Cell LC | T2bN0M0 | IIa | ✓ | | LC Focused Array |
| 566 | 71 | Male | Squamous Cell LC | T3NOMO | IIb | ✓ | | LC Focused Array |
| 567 | 53 | Male | Squamous Cell LC | T3NOMO | IIb | ✓ | ✓ | LC Focused Array |
| 568 | 54 | Male | Squamous Cell LC | T1bN2M0 | IIIa | ✓ | | LC Focused Array; ELISA |
| 569 | 76 | Female | Squamous Cell LC | T4N2M0 | IIIb | ✓ | | LC Focused Array; ELISA |
| 570 | 51 | Male | Squamous Cell LC | T2bN2M0 | IIIa | ✓ | ✓ | LC Focused Array; ELISA |
| 571 | 72 | Male | Squamous Cell LC | T3N1M0 | IIIa | ✓ | | LC Focused Array; ELISA |
| 572 | 57 | Male | Squamous Cell LC | T2aN2M0 | IIIa | ✓ | ✓ | LC Focused Array; ELISA |
| 573 | 73 | Male | Squamous Cell LC | T4N1M0 | IIIa | ✓ | | LC Focused Array; ELISA |
| 574 | 66 | Male | Squamous Cell LC | T2aN2M0 | IIIa | ✓ | | LC Focused Array; ELISA |
| 575 | 64 | Male | Squamous Cell LC | T2bN2M0 | IIIa | ✓ | ✓ | LC Focused Array; ELISA |
| 576 | 61 | Female | Squamous Cell LC | T1bN3M0 | IIIb | | | LC Focused Array; ELISA |
| 577 | 62 | Male | Squamous Cell LC | T2bN3M0 | IIIb | ✓ | ✓ | LC Focused Array; ELISA |
| 578 | 61 | Male | Squamous Cell LC | T4N2M0 | IIIb | ✓ | | LC Focused Array; ELISA |
| 579 | 70 | Male | Squamous Cell LC | T1aN2M0 | IIIa | | | LC Focused Array; ELISA |
| 580 | 47 | Male | Squamous Cell LC | T4N1M0 | IIIa | | | LC Focused Array; ELISA |
| 581 | 54 | Male | Squamous Cell LC | T1cN2M0 | IIIa | ✓ | ✓ | LC Focused Array; ELISA |
| 582 | 54 | Male | Squamous Cell LC | T1bN2M0 | IIIa | ✓ | | LC Focused Array; ELISA |
| 583 | 51 | Male | Squamous Cell LC | T4N2M0 | IIIb | ✓ | ✓ | LC Focused Array; ELISA |
| 584 | 64 | Male | Squamous Cell LC | T1bN3M0 | IIIb | ✓ | | LC Focused Array; ELISA |
| 585 | 60 | Male | Squamous Cell LC | T3N2M0 | IIIb | ✓ | | LC Focused Array; ELISA |
| 586 | 58 | Male | Squamous Cell LC | T2bN2M0 | IIIa | ✓ | | LC Focused Array |
| 587 | 58 | Male | Squamous Cell LC | T4aN0M0 | IIIa | ✓ | | LC Focused Array |
| 588 | 71 | Male | Squamous Cell LC | T2aN2M0 | IIIa | ✓ | | LC Focused Array |
| 589 | 64 | Male | Squamous Cell LC | T2bN2M0 | IIIa | ✓ | | LC Focused Array |
| 590 | 73 | Male | Squamous Cell LC | T4N2M0 | IIIb | ✓ | | LC Focused Array |
| 591 | 67 | Male | Squamous Cell LC | T2bN3M0 | IIIb | ✓ | | LC Focused Array |
| 592 | 60 | Male | Squamous Cell LC | T2aN3M0 | IIIb | ✓ | ✓ | LC Focused Array |
| 593 | 62 | Male | Squamous Cell LC | T3N1M0 | IIIa | ✓ | ✓ | LC Focused Array |
| 594 | 70 | Male | Squamous Cell LC | T4N2M0 | IIIb | | | LC Focused Array |
| 595 | 63 | Male | Squamous Cell LC | T1bN3M0 | IIIb | ✓ | ✓ | LC Focused Array |
| 596 | 65 | Male | Squamous Cell LC | T2bN3M0 | IIIb | ✓ | | LC Focused Array |
| 597 | 48 | Male | Squamous Cell LC | T3N1M0 | IIIa | ✓ | ✓ | LC Focused Array |
| 598 | 63 | Male | Squamous Cell LC | T4N0M0 | IIIa | ✓ | | LC Focused Array |
| 599 | 39 | Female | Squamous Cell LC | T4N0M0 | IIIa | ✓ | ✓ | LC Focused Array |
| 600 | 57 | Male | Squamous Cell LC | T2aN2M0 | IIIa | ✓ | | LC Focused Array |
| 601 | 70 | Male | Squamous Cell LC | T2bN2M0 | IIIa | ✓ | ✓ | LC Focused Array |
| 602 | 76 | Male | Squamous Cell LC | T2aN2M0 | IIIa | ✓ | | LC Focused Array |
| 603 | 70 | Male | Squamous Cell LC | T1bN2M0 | IIIa | ✓ | | LC Focused Array |
| 604 | 61 | Male | Squamous Cell LC | T1aN2M0 | IIIa | ✓ | ✓ | LC Focused Array |
| 605 | 61 | Male | Squamous Cell LC | T2aN2M0 | IIIa | ✓ | | LC Focused Array |
| 606 | 70 | Male | Squamous Cell LC | T2bN3M0 | IIIb | ✓ | | LC Focused Array |
| 607 | 69 | Male | Squamous Cell LC | T4N2M0 | IIIb | ✓ | | LC Focused Array |
| 608 | 71 | Male | Squamous Cell LC | T2aN3M0 | IIIb | ✓ | | LC Focused Array |
| 609 | 65 | Male | Squamous Cell LC | T4N2M0 | IIIb | ✓ | | LC Focused Array |
| 610 | 70 | Male | Squamous Cell LC | T2aN2M0 | IIIa | ✓ | | LC Focused Array |
| 611 | 61 | Male | Squamous Cell LC | T1bN3M0 | IIIb | ✓ | | LC Focused Array |
| 612 | 52 | Male | Squamous Cell LC | T4N2M0 | IIIb | ✓ | ✓ | LC Focused Array |
| 613 | 66 | Male | Squamous Cell LC | T2aN2M0 | IIIa | ✓ | | LC Focused Array |
| 614 | 66 | Male | Squamous Cell LC | T1cN3M0 | IIIb | ✓ | | LC Focused Array |
| 615 | 60 | Male | Squamous Cell LC | T2aN2M0 | IIIa | ✓ | | LC Focused Array |
| 616 | 54 | Male | Squamous Cell LC | T2aN2M0 | IIIa | ✓ | ✓ | LC Focused Array |
| 617 | 62 | Male | Squamous Cell LC | T3N2M1c | IV | ✓ | | LC Focused Array |
| 618 | 69 | Male | Squamous Cell LC | T4N2M1c | IV | ✓ | | LC Focused Array |
| 619 | 62 | Male | Squamous Cell LC | T3N2M1b | IV | ✓ | | LC Focused Array |
| 620 | 65 | Male | Squamous Cell LC | T4N2M1c | IV | ✓ | | LC Focused Array |
| 621 | 70 | Male | Squamous Cell LC | T4N3M1a | IV | ✓ | | LC Focused Array |
| 622 | 75 | Male | Squamous Cell LC | T3N2M1b | IV | ✓ | | LC Focused Array |
| 623 | 49 | Male | Squamous Cell LC | T4N2M1b | IV | ✓ | | LC Focused Array |
| 624 | 62 | Male | Squamous Cell LC | T3N3M1c | IV | ✓ | | LC Focused Array |
| 625 | 56 | Female | Squamous Cell LC | T3N3M1c | IV | | | LC Focused Array; ELISA |
| 626 | 56 | Female | Squamous Cell LC | T4N2M1c | IV | | | LC Focused Array |
| 627 | 61 | Male | Squamous Cell LC | T4N2M1b | IV | ✓ | | LC Focused Array; ELISA |
| 628 | 50 | Male | Squamous Cell LC | T4N3M1c | IV | ✓ | ✓ | LC Focused Array |
| 629 | 59 | Male | Squamous Cell LC | T3N2M1b | IV | | | LC Focused Array |
| 630 | 72 | Male | Squamous Cell LC | T4N2M1b | IV | ✓ | | LC Focused Array |
| 631 | 58 | Male | Squamous Cell LC | T3N2M1b | IV | ✓ | | LC Focused Array |
| 632 | 78 | Male | Squamous Cell LC | T4N2M1b | IV | ✓ | | LC Focused Array |
| 633 | 59 | Male | Squamous Cell LC | T4aN3M1c | IV | ✓ | ✓ | LC Focused Array |
| 634 | 59 | Male | Squamous Cell LC | T4N2M1c | IV | | | LC Focused Array |
| 635 | 59 | Male | Squamous Cell LC | T4N2M1b | IV | ✓ | | LC Focused Array |
| 636 | 59 | Male | Squamous Cell LC | T3N3M1a | IV | | | LC Focused Array |
| 637 | 46 | Male | Squamous Cell LC | T3N3M1a | IV | ✓ | ✓ | LC Focused Array |
| 638 | 68 | Female | Squamous Cell LC | T4N2M1c | IV | ✓ | | LC Focused Array |
| 639 | 58 | Male | Squamous Cell LC | T4N3M1c | IV | ✓ | | LC Focused Array |
| 640 | 77 | Male | Squamous Cell LC | T2bN3M1c | IV | ✓ | | LC Focused Array |
| 641 | 67 | Male | Squamous Cell LC | T4N3M1b | IV | ✓ | | LC Focused Array |
| 642 | 59 | Male | Squamous Cell LC | T2bN3M1b | IV | ✓ | | LC Focused Array |
| 643 | 61 | Male | Squamous Cell LC | T4N3M1b | IV | ✓ | | LC Focused Array |
| 644 | 54 | Male | Squamous Cell LC | T2aN3M1c | IV | ✓ | ✓ | LC Focused Array |
| 645 | 74 | Male | Squamous Cell LC | T3N2M1b | IV | ✓ | | LC Focused Array |
| 646 | 70 | Male | Squamous Cell LC | T4N2M1b | IV | ✓ | | LC Focused Array |
| 647 | 47 | Male | Rectal Cancer | T2aN0M0 | Ib | ✓ | ✓ | LC Focused Array |
| 648 | 18 | Female | Rectal Cancer | T2aN0M0 | Ib | | | LC Focused Array |
| 649 | 55 | Male | Rectal Cancer | T1aN0M0 | Ia | ✓ | | LC Focused Array |
| 650 | 58 | Male | Rectal Cancer | T1cN0M0 | Ia | | | LC Focused Array |
| 651 | 58 | Female | Rectal Cancer | T1aN0M0 | Ia | | | LC Focused Array |
| 652 | 62 | Male | Rectal Cancer | T1cN0M0 | Ia | ✓ | ✓ | LC Focused Array |
| 653 | 61 | Female | Rectal Cancer | T2bN0M0 | IIa | | | LC Focused Array |
| 654 | 66 | Male | Rectal Cancer | T2bN0M0 | IIa | | | LC Focused Array |
| 655 | 62 | Female | Rectal Cancer | T2bN0M0 | IIa | | | LC Focused Array |
| 656 | 66 | Female | Rectal Cancer | T2bN0M0 | IIa | | | LC Focused Array |
| 657 | 53 | Male | Rectal Cancer | T3N0M0 | IIb | ✓ | ✓ | LC Focused Array |
| 658 | 44 | Male | Rectal Cancer | T3N0M0 | IIb | ✓ | ✓ | LC Focused Array |
| 659 | 70 | Female | Rectal Cancer | T2aN1MO | IIb | | | LC Focused Array |
| 660 | 45 | Male | Rectal Cancer | T2bN1MO | IIb | ✓ | | LC Focused Array |
| 661 | 62 | Female | Rectal Cancer | T2bN1MO | IIb | | | LC Focused Array |
| 662 | 65 | Male | Rectal Cancer | T2aN2M0 | IIIa | ✓ | | LC Focused Array |
| 663 | 58 | Female | Rectal Cancer | T1aN2M0 | IIIa | | | LC Focused Array |
| 664 | 60 | Male | Rectal Cancer | T2aN2M0 | IIIa | ✓ | | LC Focused Array |
| 665 | 41 | Male | Rectal Cancer | T2bN2M0 | IIIa | ✓ | ✓ | LC Focused Array |
| 666 | 76 | Male | Rectal Cancer | T2aN2M0 | IIIa | ✓ | | LC Focused Array |
| 667 | 61 | Female | Rectal Cancer | T1bN2M0 | IIIa | | | LC Focused Array |
| 668 | 52 | Male | Rectal Cancer | T2bN2M0 | IIIa | ✓ | | LC Focused Array |
| 669 | 63 | Female | Rectal Cancer | T2aN2M0 | IIIa | | | LC Focused Array |
| 670 | 71 | Male | Rectal Cancer | T4N2M0 | IIIb | | | LC Focused Array |
| 671 | 56 | Male | Rectal Cancer | T3N2M0 | IIIb | ✓ | ✓ | LC Focused Array |
| 672 | 75 | Male | Rectal Cancer | T3N2M0 | IIIb | ✓ | | LC Focused Array |
| 673 | 63 | Female | Rectal Cancer | T2bN3M0 | IIIb | | | LC Focused Array |
| 674 | 65 | Male | Rectal Cancer | T4N2M0 | IIIb | | | LC Focused Array |
| 675 | 61 | Female | Rectal Cancer | T2aN3M0 | IIIb | ✓ | | LC Focused Array |
| 676 | 57 | Male | Rectal Cancer | T3N2M1 | IV | | | LC Focused Array |
| 677 | 73 | Male | Rectal Cancer | T4N2M1 | IV | ✓ | | LC Focused Array |
| 678 | 74 | Female | Rectal Cancer | T4N3M1 | IV | | | LC Focused Array |
| 679 | 69 | Male | Rectal Cancer | T3N2M1 | IV | ✓ | | LC Focused Array |
| 680 | 62 | Female | Rectal Cancer | T4N2M1 | IV | | | LC Focused Array |
| 681 | 50 | Female | Liver Cancer | | A1 (BCLC) | | | LC Focused Array |
| 682 | 43 | Male | Liver Cancer | | A1 (BCLC) | ✓ | | LC Focused Array |
| 683 | 46 | Male | Liver Cancer | | A2 (BCLC) | | | LC Focused Array |
| 684 | 59 | Male | Liver Cancer | | A2 (BCLC) | | | LC Focused Array |
| 685 | 74 | Male | Liver Cancer | | A2 (BCLC) | ✓ | | LC Focused Array |
| 686 | 74 | Female | Liver Cancer | | A3 (BCLC) | | | LC Focused Array |
| 687 | 56 | Male | Liver Cancer | | A4 (BCLC) | ✓ | ✓ | LC Focused Array |
| 688 | 39 | Male | Liver Cancer | | A4 (BCLC) | | | LC Focused Array |
| 689 | 73 | Male | Liver Cancer | | A4 (BCLC) | | | LC Focused Array |
| 690 | 61 | Female | Liver Cancer | | A4 (BCLC) | | | LC Focused Array |
| 691 | 62 | Female | Liver Cancer | | B (BCLC) | | | LC Focused Array |
| 692 | 51 | Male | Liver Cancer | | B (BCLC) | ✓ | ✓ | LC Focused Array |
| 693 | 69 | Male | Liver Cancer | | B (BCLC) | ✓ | | LC Focused Array |
| 694 | 67 | Male | Liver Cancer | | B (BCLC) | | | LC Focused Array |
| 695 | 52 | Male | Liver Cancer | | B (BCLC) | ✓ | | LC Focused Array |
| 696 | 47 | Male | Liver Cancer | | B (BCLC) | ✓ | | LC Focused Array |
| 697 | 31 | Male | Liver Cancer | | B (BCLC) | ✓ | ✓ | LC Focused Array |
| 698 | 52 | Male | Liver Cancer | | B (BCLC) | ✓ | | LC Focused Array |
| 699 | 51 | Male | Liver Cancer | | B (BCLC) | | | LC Focused Array |
| 700 | 43 | Male | Liver Cancer | | B (BCLC) | | | LC Focused Array |
| 701 | 46 | Male | Liver Cancer | | B (BCLC) | ✓ | ✓ | LC Focused Array |
| 702 | 59 | Male | Liver Cancer | | B (BCLC) | | | LC Focused Array |
| 703 | 56 | Male | Liver Cancer | | B (BCLC) | | | LC Focused Array |
| 704 | 58 | Male | Liver Cancer | | B (BCLC) | ✓ | | LC Focused Array |
| 705 | 56 | Female | Liver Cancer | | B (BCLC) | | | LC Focused Array |
| 706 | 68 | Male | Liver Cancer | | B (BCLC) | ✓ | | LC Focused Array |
| 707 | 61 | Female | Liver Cancer | | B (BCLC) | | | LC Focused Array |
| 708 | 66 | Male | Liver Cancer | | B (BCLC) | ✓ | | LC Focused Array |
| 709 | 48 | Male | Liver Cancer | | B (BCLC) | ✓ | ✓ | LC Focused Array |
| 710 | 72 | Male | Liver Cancer | | B (BCLC) | ✓ | | LC Focused Array |
| 711 | 65 | Male | Liver Cancer | | B (BCLC) | | | LC Focused Array |
| 712 | 65 | Male | Liver Cancer | | B (BCLC) | | | LC Focused Array |
| 713 | 62 | Female | Liver Cancer | | B (BCLC) | | | LC Focused Array |
| 714 | 72 | Male | Liver Cancer | | B (BCLC) | | | LC Focused Array |
| 715 | 69 | Male | Liver Cancer | | B (BCLC) | ✓ | | LC Focused Array |
| 716 | 58 | Male | Liver Cancer | | B (BCLC) | ✓ | ✓ | LC Focused Array |
| 717 | 74 | Male | Liver Cancer | | C (BCLC) | ✓ | | LC Focused Array |
| 718 | 48 | Female | Liver Cancer | | C (BCLC) | | | LC Focused Array |
| 719 | 39 | Male | Liver Cancer | | C (BCLC) | ✓ | | LC Focused Array |
| 720 | 34 | Male | Liver Cancer | | C (BCLC) | | | LC Focused Array |
| 721 | 42 | Male | Liver Cancer | | C (BCLC) | ✓ | ✓ | LC Focused Array |
| 722 | 51 | Male | Liver Cancer | | C (BCLC) | ✓ | ✓ | LC Focused Array |
| 723 | 65 | Male | Liver Cancer | | C (BCLC) | | | LC Focused Array |
| 724 | 71 | Female | Liver Cancer | | C (BCLC) | | | LC Focused Array |
| 725 | 34 | Male | Liver Cancer | | C (BCLC) | ✓ | ✓ | LC Focused Array |
| 726 | 59 | Male | Liver Cancer | | C (BCLC) | ✓ | | LC Focused Array |
| 727 | 75 | Male | Liver Cancer | | C (BCLC) | | | LC Focused Array |
| 728 | 62 | Male | Liver Cancer | | C (BCLC) | | | LC Focused Array |
| 729 | 77 | Male | Liver Cancer | | C (BCLC) | ✓ | | LC Focused Array |
| 730 | 47 | Female | Liver Cancer | | C (BCLC) | | | LC Focused Array |
| 731 | 53 | Male | Liver Cancer | | C (BCLC) | ✓ | ✓ | LC Focused Array |
| 732 | 60 | Female | Liver Cancer | | C (BCLC) | | | LC Focused Array |
| 733 | 72 | Female | Liver Cancer | | C (BCLC) | | | LC Focused Array |
| 734 | 72 | Male | Liver Cancer | | C (BCLC) | ✓ | | LC Focused Array |
| 735 | 55 | Male | Liver Cancer | | C (BCLC) | ✓ | | LC Focused Array |
| 736 | 67 | Male | Liver Cancer | | C (BCLC) | | | LC Focused Array |
| 737 | 51 | Male | Liver Cancer | | C (BCLC) | | | LC Focused Array |
| 738 | 50 | Male | Liver Cancer | | C (BCLC) | ✓ | | LC Focused Array |
| 739 | 27 | Male | Liver Cancer | | C (BCLC) | | | LC Focused Array |
| 740 | 60 | Male | Liver Cancer | | D (BCLC) | | | LC Focused Array |
| 741 | 36 | Male | Liver Cancer | | D (BCLC) | ✓ | ✓ | LC Focused Array |
| 742 | 71 | Male | Liver Cancer | | D (BCLC) | | | LC Focused Array |
| 743 | 71 | Male | Liver Cancer | | D (BCLC) | ✓ | | LC Focused Array |
| 744 | 44 | Male | Liver Cancer | | D (BCLC) | ✓ | ✓ | LC Focused Array |
| 745 | 63 | Male | Liver Cancer | | D (BCLC) | | | LC Focused Array |
| 746 | 52 | Male | Liver Cancer | | D (BCLC) | ✓ | | LC Focused Array |
| 747 | 34 | Female | Cervical Cancer | | Ia1 (FIGO) | ✓ | ✓ | LC Focused Array |
| 748 | 44 | Female | Cervical Cancer | | Ia1 (FIGO) | | | LC Focused Array |
| 749 | 42 | Female | Cervical Cancer | | Ia2 (FIGO) | | | LC Focused Array |
| 750 | 55 | Female | Cervical Cancer | | Ia2 (FIGO) | | | LC Focused Array |
| 751 | 42 | Female | Cervical Cancer | | Ia2 (FIGO) | | | LC Focused Array |
| 752 | 61 | Female | Cervical Cancer | | Ib1 (FIGO) | | | LC Focused Array |
| 753 | 52 | Female | Cervical Cancer | | Ib1 (FIGO) | | | LC Focused Array |
| 754 | 52 | Female | Cervical Cancer | | Ib1 (FIGO) | | | LC Focused Array |
| 755 | 46 | Female | Cervical Cancer | | Ib1 (FIGO) | | | LC Focused Array |
| 756 | 44 | Female | Cervical Cancer | | Ib1 (FIGO) | | | LC Focused Array |
| 757 | 49 | Female | Cervical Cancer | | Ib1 (FIGO) | | | LC Focused Array |
| 758 | 29 | Female | Cervical Cancer | | IIa (FIGO) | ✓ | ✓ | LC Focused Array |
| 759 | 24 | Female | Cervical Cancer | | IIa (FIGO) | | | LC Focused Array |
| 760 | 50 | Female | Cervical Cancer | | IIa (FIGO) | | | LC Focused Array |
| 761 | 54 | Female | Cervical Cancer | | IIa (FIGO) | | | LC Focused Array |
| 762 | 52 | Female | Cervical Cancer | | IIb (FIGO) | | | LC Focused Array |
| 763 | 47 | Female | Cervical Cancer | | IIb (FIGO) | | | LC Focused Array |
| 764 | 52 | Female | Cervical Cancer | | IIb (FIGO) | | | LC Focused Array |
| 765 | 46 | Female | Cervical Cancer | | IIb (FIGO) | | | LC Focused Array |
| 766 | 38 | Female | Cervical Cancer | | IIb (FIGO) | ✓ | ✓ | LC Focused Array |
| 767 | 39 | Female | Cervical Cancer | | IIIa (FIGO) | | | LC Focused Array |
| 768 | 46 | Female | Cervical Cancer | | IIIa (FIGO) | | | LC Focused Array |
| 769 | 41 | Female | Cervical Cancer | | IIIa (FIGO) | | | LC Focused Array |
| 770 | 44 | Female | Cervical Cancer | | IIIb (FIGO) | | | LC Focused Array |
| 771 | 59 | Female | Cervical Cancer | | IIIb (FIGO) | | | LC Focused Array |
| 772 | 64 | Female | Cervical Cancer | | IIIb (FIGO) | | | LC Focused Array |
| 773 | 42 | Female | Cervical Cancer | | IIIb (FIGO) | | | LC Focused Array |
| 774 | 59 | Male | Esophagus Cancer | T1aN0M0 | Ia | ✓ | ✓ | LC Focused Array |
| 775 | 54 | Male | Esophagus Cancer | T1cN0M0 | Ia | ✓ | ✓ | LC Focused Array |
| 776 | 60 | Male | Esophagus Cancer | T1bN1M0 | Ia | | | LC Focused Array |
| 777 | 59 | Female | Esophagus Cancer | T1aN0M0 | Ia | | | LC Focused Array |
| 778 | 64 | Female | Esophagus Cancer | T2aN0M0 | Ib | | | LC Focused Array |
| 779 | 62 | Male | Esophagus Cancer | T2aN0M0 | Ib | | | LC Focused Array |
| 780 | 68 | Male | Esophagus Cancer | T2bN0M0 | IIa | | | LC Focused Array |
| 781 | 56 | Male | Esophagus Cancer | T2bN0M0 | IIa | ✓ | | LC Focused Array |
| 782 | 74 | Male | Esophagus Cancer | T2aN1MO | IIb | ✓ | | LC Focused Array |
| 783 | 53 | Female | Esophagus Cancer | T3N0M0 | IIb | | | LC Focused Array |
| 784 | 62 | Male | Esophagus Cancer | T3N0M0 | IIb | ✓ | ✓ | LC Focused Array |
| 785 | 51 | Male | Esophagus Cancer | T2aN1MO | IIb | ✓ | ✓ | LC Focused Array |
| 786 | 60 | Male | Esophagus Cancer | T2bN1MO | IIb | ✓ | | LC Focused Array |
| 787 | 79 | Male | Esophagus Cancer | T2bN1MO | IIb | | | LC Focused Array |
| 788 | 53 | Male | Esophagus Cancer | T3N0M0 | IIb | ✓ | ✓ | LC Focused Array |
| 789 | 58 | Male | Esophagus Cancer | T2bN2M0 | IIIa | | | LC Focused Array |
| 790 | 64 | Male | Esophagus Cancer | T4aN0M0 | IIIa | ✓ | | LC Focused Array |
| 791 | 70 | Male | Esophagus Cancer | T2aN2M0 | IIIa | ✓ | | LC Focused Array |
| 792 | 63 | Male | Esophagus Cancer | T2bN2M0 | IIIa | | | LC Focused Array |
| 793 | 68 | Male | Esophagus Cancer | T2bN2M0 | IIIa | ✓ | ✓ | LC Focused Array |
| 794 | 63 | Male | Esophagus Cancer | T2aN2M0 | IIIa | | | LC Focused Array |
| 795 | 60 | Female | Esophagus Cancer | T1bN2M0 | IIIa | | | LC Focused Array |
| 796 | 59 | Male | Esophagus Cancer | T3N1M0 | IIIa | | | LC Focused Array |
| 797 | 68 | Male | Esophagus Cancer | T3N1M0 | IIIa | ✓ | | LC Focused Array |
| 798 | 57 | Female | Esophagus Cancer | T2aN2M0 | IIIa | ✓ | | LC Focused Array |
| 799 | 65 | Male | Esophagus Cancer | T4N1M0 | IIIa | ✓ | | LC Focused Array |
| 800 | 80 | Male | Esophagus Cancer | T3N1M0 | IIIa | ✓ | | LC Focused Array |
| 801 | 53 | Male | Esophagus Cancer | T4N0M0 | IIIa | ✓ | ✓ | LC Focused Array |
| 802 | 64 | Male | Esophagus Cancer | T4N0M0 | IIIa | | | LC Focused Array |
| 803 | 52 | Female | Esophagus Cancer | T2aN2M0 | IIIa | | | LC Focused Array |
| 804 | 46 | Male | Esophagus Cancer | T2bN2M0 | IIIa | ✓ | ✓ | LC Focused Array |
| 805 | 49 | Female | Esophagus Cancer | T4N2M0 | IIIb | | | LC Focused Array |
| 806 | 69 | Male | Esophagus Cancer | T2bN3M0 | IIIb | ✓ | | LC Focused Array |
| 807 | 64 | Male | Esophagus Cancer | T2aN3M0 | IIIb | | | LC Focused Array |
| 808 | 70 | Male | Esophagus Cancer | T4N2M0 | IIIb | | | LC Focused Array |
| 809 | 61 | Male | Esophagus Cancer | T1bN3M0 | IIIb | ✓ | ✓ | LC Focused Array |
| 810 | 60 | Male | Esophagus Cancer | T2bN3M0 | IIIb | ✓ | | LC Focused Array |
| 811 | 74 | Female | Esophagus Cancer | T1bN3M0 | IIIb | | | LC Focused Array |
| 812 | 50 | Male | Esophagus Cancer | T2bN3M0 | IIIb | | | LC Focused Array |
| 813 | 51 | Male | Esophagus Cancer | T4N2M0 | IIIb | | | LC Focused Array |
| 814 | 60 | Male | Esophagus Cancer | T4N2M1 | IV | ✓ | | LC Focused Array |
| 815 | 58 | Female | Esophagus Cancer | T4N3M1 | IV | | | LC Focused Array |
| 816 | 61 | Male | Esophagus Cancer | T3N2M1 | IV | ✓ | | LC Focused Array |
| 817 | 63 | Female | Esophagus Cancer | T4N2M1 | IV | | | LC Focused Array |
| 818 | 52 | Female | Esophagus Cancer | T3N3M1 | IV | | | LC Focused Array |
| 819 | 37 | Female | Esophagus Cancer | T3N3M1 | IV | | | LC Focused Array |
| 820 | 73 | Male | Esophagus Cancer | T4N2M1 | IV | ✓ | | LC Focused Array |
| 821 | 65 | Male | Esophagus Cancer | T4N2M1 | IV | ✓ | | LC Focused Array |
| 822 | 56 | Male | Gastric Cancer | T1aN0M0 | Ia | | | LC Focused Array |
| 823 | 56 | Male | Gastric Cancer | T1bN0M0 | Ia | | | LC Focused Array |
| 824 | 57 | Male | Gastric Cancer | T1aN0M0 | Ia | ✓ | ✓ | LC Focused Array |
| 825 | 58 | Male | Gastric Cancer | T1aN0M0 | Ia | ✓ | | LC Focused Array |
| 826 | 52 | Male | Gastric Cancer | T2aN0M0 | Ib | ✓ | ✓ | LC Focused Array |
| 827 | 51 | Female | Gastric Cancer | T2aN0M0 | Ib | | | LC Focused Array |
| 828 | 74 | Male | Gastric Cancer | T2aN0M0 | Ib | ✓ | | LC Focused Array |
| 829 | 70 | Male | Gastric Cancer | T2aN0M0 | Ib | | | LC Focused Array |
| 830 | 76 | Male | Gastric Cancer | T2aN0M0 | Ib | | | LC Focused Array |
| 831 | 63 | Male | Gastric Cancer | T2aN1MO | IIb | | | LC Focused Array |
| 832 | 64 | Male | Gastric Cancer | T3N0M0 | IIb | ✓ | ✓ | LC Focused Array |
| 833 | 70 | Male | Gastric Cancer | T3N0M0 | IIb | ✓ | | LC Focused Array |
| 834 | 65 | Male | Gastric Cancer | T2aN1MO | IIb | ✓ | | LC Focused Array |
| 835 | 63 | Male | Gastric Cancer | T2bN1MO | IIb | | | LC Focused Array |
| 836 | 47 | Male | Gastric Cancer | T2bN1MO | IIb | ✓ | ✓ | LC Focused Array |
| 837 | 66 | Male | Gastric Cancer | T3N0M0 | IIb | ✓ | | LC Focused Array |
| 838 | 55 | Male | Gastric Cancer | T3NOMO | IIb | ✓ | ✓ | LC Focused Array |
| 839 | 57 | Male | Gastric Cancer | T1bN2M0 | IIIa | | | LC Focused Array |
| 840 | 62 | Male | Gastric Cancer | T1aN2M0 | IIIa | ✓ | | LC Focused Array |
| 841 | 60 | Male | Gastric Cancer | T2aN2M0 | IIIa | ✓ | ✓ | LC Focused Array |
| 842 | 74 | Male | Gastric Cancer | T2bN2M0 | IIIa | ✓ | | LC Focused Array |
| 843 | 67 | Male | Gastric Cancer | T4N1M0 | IIIa | | | LC Focused Array |
| 844 | 59 | Male | Gastric Cancer | T2aN2M0 | IIIa | ✓ | ✓ | LC Focused Array |
| 845 | 69 | Female | Gastric Cancer | T2bN2M0 | IIIa | | | LC Focused Array |
| 846 | 66 | Male | Gastric Cancer | T4N1M0 | IIIa | ✓ | | LC Focused Array |
| 847 | 59 | Male | Gastric Cancer | T1aN2M0 | IIIa | ✓ | | LC Focused Array |
| 848 | 63 | Female | Gastric Cancer | T1bN2M0 | IIIa | | | LC Focused Array |
| 849 | 46 | Female | Gastric Cancer | T3N1M0 | IIIa | ✓ | ✓ | LC Focused Array |
| 850 | 78 | Male | Gastric Cancer | T4N1M0 | IIIa | ✓ | | LC Focused Array |
| 851 | 78 | Male | Gastric Cancer | T2aN3M0 | IIIb | | | LC Focused Array |
| 852 | 55 | Male | Gastric Cancer | T4N2M0 | IIIb | ✓ | | LC Focused Array |
| 853 | 58 | Male | Gastric Cancer | T2aN3M0 | IIIb | | | LC Focused Array |
| 854 | 47 | Female | Gastric Cancer | T3N2M0 | IIIb | | | LC Focused Array |
| 855 | 59 | Male | Gastric Cancer | T3N2M0 | IIIb | ✓ | | LC Focused Array |
| 856 | 50 | Male | Gastric Cancer | T4N2M0 | IIIb | ✓ | ✓ | LC Focused Array |
| 857 | 72 | Male | Gastric Cancer | T2bN3M0 | IIIb | | | LC Focused Array |
| 858 | 67 | Male | Gastric Cancer | T1bN3M0 | IIIb | ✓ | | LC Focused Array |
| 859 | 64 | Female | Gastric Cancer | T1bN2M0 | IIIb | | | LC Focused Array |
| 860 | 54 | Male | Gastric Cancer | T2aN3M0 | IIIb | ✓ | | LC Focused Array |
| 861 | 75 | Male | Gastric Cancer | T3N2M0 | IIIb | | | LC Focused Array |
| 862 | 49 | Female | Gastric Cancer | T3N2M0 | IIIb | ✓ | ✓ | LC Focused Array |
| 863 | 56 | Male | Gastric Cancer | T4N2M0 | IIIb | ✓ | | LC Focused Array |
| 864 | 62 | Female | Gastric Cancer | T4N2M0 | IIIb | | | LC Focused Array |
| 865 | 72 | Female | Gastric Cancer | T2aN3M0 | IIIb | | | LC Focused Array |
| 866 | 56 | Female | Gastric Cancer | T4N2M0 | IIIb | ✓ | | LC Focused Array |
| 867 | 44 | Male | Gastric Cancer | T2bN3M1 | IV | ✓ | ✓ | LC Focused Array |
| 868 | 80 | Female | Gastric Cancer | T3N2M1 | IV | | | LC Focused Array |
| 869 | 47 | Male | Gastric Cancer | T3N3M1 | IV | ✓ | ✓ | LC Focused Array |
| 870 | 69 | Male | Gastric Cancer | T4N2M1 | IV | ✓ | | LC Focused Array |
| 871 | 66 | Male | Gastric Cancer | T4N2M1 | IV | | | LC Focused Array |
| 872 | 47 | Female | Gastric Cancer | T4N3M1 | IV | | | LC Focused Array |
| 873 | 64 | Male | Healthy | | | ✓ | | ELISA |
| 874 | 56 | Male | Healthy | | | ✓ | | ELISA |
| 875 | 63 | Male | Healthy | | | ✓ | | ELISA |
| 876 | 49 | Male | Healthy | | | ✓ | | ELISA |
| 877 | 33 | Female | Healthy | | | ✓ | ✓ | ELISA |
| 878 | 38 | Male | Healthy | | | ✓ | ✓ | ELISA |
| 879 | 47 | Female | Healthy | | | | | ELISA |
| 880 | 48 | Male | Healthy | | | ✓ | | ELISA |
| 881 | 74 | Male | Healthy | | | ✓ | | ELISA |
| 882 | 45 | Male | Healthy | | | ✓ | | ELISA |
| 883 | 34 | Male | Healthy | | | ✓ | ✓ | ELISA |
| 884 | 52 | Male | Healthy | | | ✓ | | ELISA |
| 885 | 46 | Male | Healthy | | | ✓ | ✓ | ELISA |
| 886 | 43 | Female | Healthy | | | | | ELISA |
| 887 | 39 | Female | Healthy | | | | | ELISA |
| 888 | 45 | Female | Healthy | | | ✓ | ✓ | ELISA |
| 889 | 67 | Male | Healthy | | | ✓ | | ELISA |
| 890 | 52 | Female | Healthy | | | | | ELISA |
| 891 | 42 | Female | Healthy | | | | | ELISA |
| 892 | 66 | Male | Healthy | | | ✓ | | ELISA |
| 893 | 56 | Male | Healthy | | | ✓ | | ELISA |
| 894 | 51 | Male | Healthy | | | ✓ | ✓ | ELISA |
| 895 | 44 | Male | Healthy | | | ✓ | | ELISA |
| 896 | 53 | Male | Healthy | | | ✓ | | ELISA |
| 897 | 46 | Female | Healthy | | | ✓ | ✓ | ELISA |
| 898 | 66 | Male | Healthy | | | ✓ | | ELISA |
| 899 | 55 | Male | Healthy | | | | | ELISA |
| 900 | 43 | Male | Healthy | | | | | ELISA |
| 901 | 44 | Male | Healthy | | | ✓ | | ELISA |
| 902 | 54 | Female | Healthy | | | ✓ | ✓ | ELISA |
| 903 | 38 | Female | Healthy | | | | | ELISA |
| 904 | 72 | Male | Healthy | | | ✓ | | ELISA |
| 905 | 43 | Male | Healthy | | | ✓ | ✓ | ELISA |
| 906 | 70 | Female | Healthy | | | | | ELISA |
| 907 | 62 | Male | Healthy | | | ✓ | | ELISA |
| 908 | 45 | Male | Healthy | | | ✓ | ✓ | ELISA |
| 909 | 60 | Male | Healthy | | | ✓ | | ELISA |
| 910 | 53 | Female | Healthy | | | ✓ | | ELISA |
| 911 | 38 | Female | Healthy | | | ✓ | ✓ | ELISA |
| 912 | 50 | Male | Healthy | | | ✓ | | ELISA |
| 913 | 48 | Female | Healthy | | | | | ELISA |
| 914 | 44 | Male | Healthy | | | ✓ | | ELISA |
| 915 | 71 | Female | Healthy | | | | | ELISA |
| 916 | 52 | Male | Healthy | | | ✓ | | ELISA |
| 917 | 53 | Male | Healthy | | | ✓ | ✓ | ELISA |
| 918 | 61 | Female | Healthy | | | ✓ | | ELISA |
| 919 | 70 | Male | Healthy | | | ✓ | | ELISA |
| 920 | 54 | Male | Healthy | | | ✓ | | ELISA |
| 921 | 53 | Male | Healthy | | | ✓ | | ELISA |
| 922 | 84 | Male | Pneumonia | | | | | ELISA |
| 923 | 83 | Male | Pneumonia | | | | | ELISA |
| 924 | 91 | Male | Pneumonia | | | ✓ | | ELISA |
| 925 | 83 | Male | COPD | | | ✓ | | ELISA |
| 926 | 72 | Male | COPD | | | ✓ | | ELISA |
| 927 | 43 | Female | Pulmonary tuberculosis | | | | | ELISA |
| 928 | 62 | Male | Pneumonia | | | ✓ | | ELISA |
| 929 | 57 | Female | Pneumonia | | | ✓ | ✓ | ELISA |
| 930 | 62 | Female | COPD | | | ✓ | | ELISA |
| 931 | 69 | Male | COPD | | | ✓ | | ELISA |
| 932 | 56 | Female | Pneumonia | | | ✓ | ✓ | ELISA |
| 933 | 66 | Female | Pulmonary tuberculosis | | | | | ELISA |
| 934 | 52 | Male | COPD | | | ✓ | ✓ | ELISA |
| 935 | 35 | Female | Pneumonia | | | | | ELISA |
| 936 | 64 | Male | Pneumonia | | | ✓ | ✓ | ELISA |
| 937 | 71 | Male | Pneumonia | | | | | ELISA |
| 938 | 56 | Male | Pneumonia | | | ✓ | ✓ | ELISA |
| 939 | 67 | Male | Pneumonia | | | ✓ | | ELISA |
| 940 | 73 | Female | Pneumonia | | | ✓ | | ELISA |
| 941 | 64 | Male | Pneumonia | | | ✓ | ✓ | ELISA |
| 942 | 69 | Male | Pulmonary tuberculosis | | | | | ELISA |
| 943 | 62 | Male | Pulmonary tuberculosis | | | | | ELISA |
| 944 | 67 | Male | COPD | | | ✓ | | ELISA |
| 945 | 58 | Male | COPD | | | ✓ | | ELISA |
| 946 | 52 | Male | Pneumonia | | | ✓ | ✓ | ELISA |
| 947 | 66 | Male | COPD | | | ✓ | | ELISA |
| 948 | 54 | Male | Pneumonia | | | ✓ | | ELISA |
| 949 | 30 | Male | Pneumonia | | | ✓ | ✓ | ELISA |
| 950 | 80 | Male | Pneumonia | | | ✓ | | ELISA |
| 951 | 51 | Female | Pneumonia | | | | | ELISA |
| 952 | 27 | Male | Pneumonia | | | ✓ | ✓ | ELISA |
| 953 | 53 | Male | Pulmonary tuberculosis | | | | | ELISA |
| 954 | 54 | Male | Pulmonary tuberculosis | | | ✓ | ✓ | ELISA |
| 955 | 56 | Male | Pneumonia | | | | | ELISA |
| 956 | 63 | Female | Pneumonia | | | | | ELISA |
| 957 | 67 | Male | COPD | | | ✓ | | ELISA |
| 958 | 58 | Male | COPD | | | ✓ | | ELISA |
| 959 | 53 | Male | Pulmonary tuberculosis | | | ✓ | | ELISA |
| 960 | 74 | Male | Pneumonia | | | ✓ | | ELISA |
| 961 | 78 | Male | COPD | | | ✓ | | ELISA |
| 962 | 57 | Male | COPD | | | ✓ | | ELISA |
| 963 | 57 | Female | COPD | | | | | ELISA |
| 964 | 47 | Male | Pulmonary tuberculosis | | | ✓ | ✓ | ELISA |
| 965 | 44 | Male | Pneumonia | | | ✓ | ✓ | ELISA |
| 966 | 44 | Male | Pneumonia | | | ✓ | ✓ | ELISA |
| 967 | 67 | Male | Pneumonia | | | ✓ | | ELISA |
| 968 | 53 | Male | Pneumonia | | | ✓ | | ELISA |
| 969 | 39 | Male | Pneumonia | | | ✓ | ✓ | ELISA |
| 970 | 80 | Female | Pneumonia | | | | | ELISA |
| 971 | 73 | Male | Pulmonary tuberculosis | | | | | ELISA |
| 972 | 43 | Female | Pulmonary tuberculosis | | | | | ELISA |
| 973 | 53 | Female | Pulmonary tuberculosis | | | | | ELISA |
| 974 | 51 | Male | Small Cell LC | | Limited | ✓ | ✓ | ELISA |
| 975 | 56 | Male | Small Cell LC | | Limited | ✓ | | ELISA |
| 976 | 66 | Male | Small Cell LC | | Limited | ✓ | | ELISA |
| 977 | 51 | Male | Small Cell LC | | Limited | ✓ | | ELISA |
| 978 | 52 | Female | Small Cell LC | | Limited | ✓ | ✓ | ELISA |
| 979 | 76 | Male | Small Cell LC | | Limited | ✓ | | ELISA |
| 980 | 70 | Male | Small Cell LC | | Limited | ✓ | | ELISA |
| 981 | 45 | Male | Small Cell LC | | Limited | ✓ | ✓ | ELISA |
| 982 | 61 | Male | Small Cell LC | | Limited | ✓ | | ELISA |
| 983 | 60 | Male | Small Cell LC | | Limited | ✓ | | ELISA |
| 984 | 63 | Male | Small Cell LC | | Limited | ✓ | | ELISA |
| 985 | 56 | Male | Small Cell LC | | Limited | ✓ | | ELISA |
| 986 | 62 | Male | Small Cell LC | | Limited | ✓ | ✓ | ELISA |
| 987 | 51 | Male | Small Cell LC | | Limited | ✓ | ✓ | ELISA |
| 988 | 75 | Male | Small Cell LC | | Limited | ✓ | | ELISA |
| 989 | 46 | Male | Small Cell LC | | Limited | ✓ | ✓ | ELISA |
| 990 | 65 | Male | Small Cell LC | | Extensive | ✓ | ✓ | ELISA |
| 991 | 62 | Male | Small Cell LC | | Extensive | ✓ | | ELISA |
| 992 | 66 | Male | Small Cell LC | | Extensive | ✓ | | ELISA |
| 993 | 56 | Male | Small Cell LC | | Extensive | ✓ | | ELISA |
| 994 | 51 | Female | Small Cell LC | | Extensive | ✓ | ✓ | ELISA |
| 995 | 63 | Male | Small Cell LC | | Extensive | ✓ | | ELISA |
| 996 | 69 | Male | Small Cell LC | | Extensive | ✓ | | ELISA |
| 997 | 61 | Male | Small Cell LC | | Extensive | ✓ | ✓ | ELISA |
| 998 | 68 | Male | Small Cell LC | | Extensive | ✓ | | ELISA |
| 999 | 55 | Male | Small Cell LC | | Extensive | ✓ | | ELISA |
| 1000 | 67 | Male | Small Cell LC | | Extensive | ✓ | | ELISA |
| 1001 | 68 | Male | Small Cell LC | | Extensive | ✓ | | ELISA |
| 1002 | 64 | Female | Small Cell LC | | Extensive | ✓ | | ELISA |
| 1003 | 67 | Male | Small Cell LC | | Extensive | ✓ | | ELISA |
| 1004 | 65 | Male | Small Cell LC | | Extensive | ✓ | | ELISA |
| 1005 | 51 | Male | Small Cell LC | | Extensive | ✓ | ✓ | ELISA |
| 1006 | 65 | Male | Small Cell LC | | Extensive | ✓ | | ELISA |
| 1007 | 67 | Male | Small Cell LC | | Extensive | ✓ | | ELISA |
| 1008 | 56 | Male | Small Cell LC | | Extensive | ✓ | | ELISA |
| 1009 | 56 | Male | Small Cell LC | | Extensive | ✓ | | ELISA |
| 1010 | 62 | Male | Small Cell LC | | Extensive | ✓ | | ELISA |
| 1011 | 62 | Male | Small Cell LC | | Extensive | ✓ | | ELISA |
| 1012 | 48 | Male | Lung Adenocarcinoma | T1bN0M0 | Ia | ✓ | ✓ | ELISA |
| 1013 | 55 | Male | Lung Adenocarcinoma | T1aN0M0 | Ia | ✓ | | ELISA |
| 1014 | 55 | Female | Lung Adenocarcinoma | T1aN0M0 | Ia | | | ELISA |
| 1015 | 49 | Female | Lung Adenocarcinoma | T1aN0M0 | Ia | | | ELISA |
| 1016 | 52 | Male | Lung Adenocarcinoma | T2aN0M0 | Ib | ✓ | | ELISA |
| 1017 | 44 | Female | Lung Adenocarcinoma | T2aN0M0 | Ib | | | ELISA |
| 1018 | 68 | Male | Lung Adenocarcinoma | T2aN0M0 | Ib | ✓ | | ELISA |
| 1019 | 60 | Male | Lung Adenocarcinoma | T2bN0M0 | IIa | ✓ | ✓ | ELISA |
| 1020 | 59 | Male | Lung Adenocarcinoma | T2bN0M0 | IIa | ✓ | | ELISA |
| 1021 | 77 | Male | Lung Adenocarcinoma | T2bN1M0 | IIb | ✓ | | ELISA |
| 1022 | 79 | Male | Lung Adenocarcinoma | T3NOMO | IIb | | | ELISA |
| 1023 | 40 | Female | Lung Adenocarcinoma | T3N0M0 | IIb | | | ELISA |
| 1024 | 52 | Male | Lung Adenocarcinoma | T1aN2M0 | IIIa | ✓ | ✓ | ELISA |
| 1025 | 36 | Female | Lung Adenocarcinoma | T1bN2M0 | IIIa | | | ELISA |
| 1026 | 65 | Male | Lung Adenocarcinoma | T4N2M0 | IIIb | ✓ | | ELISA |
| 1027 | 75 | Male | Lung Adenocarcinoma | T2bN3M0 | IIIb | ✓ | | ELISA |
| 1028 | 66 | Male | Lung Adenocarcinoma | T4N2M1c | IV | ✓ | | ELISA |
| 1029 | 68 | Male | Lung Adenocarcinoma | T4N2M1b | IV | ✓ | | ELISA |
| 1030 | 46 | Female | Lung Adenocarcinoma | T4N3M1b | IV | | | ELISA |
| 1031 | 75 | Male | Lung Adenocarcinoma | T2aN2M1b | IV | ✓ | | ELISA |
| 1032 | 61 | Female | Lung Adenocarcinoma | T2aN3M1a | IV | | | ELISA |
| 1033 | 62 | Male | Lung Adenocarcinoma | T2bN2M1b | IV | | | ELISA |
| 1034 | 60 | Female | Lung Adenocarcinoma | T4N3M1b | IV | | | ELISA |
| 1035 | 64 | Male | Lung Adenocarcinoma | T4N3M1c | IV | | | ELISA |
| 1036 | 73 | Female | Lung Adenocarcinoma | T4N2M1c | IV | | | ELISA |
| 1037 | 58 | Female | Lung Adenocarcinoma | T2N3M1a | IV | ✓ | ✓ | ELISA |
| 1038 | 49 | Male | Lung Adenocarcinoma | T4N3M1c | IV | ✓ | | ELISA |
| 1039 | 48 | Female | Lung Adenocarcinoma | T3N2M1c | IV | | | ELISA |
| 1040 | 66 | Female | Lung Adenocarcinoma | T4N3M1b | IV | | | ELISA |
| 1041 | 60 | Male | Lung Adenocarcinoma | T2bN3M1a | IV | ✓ | ✓ | ELISA |
| 1042 | 77 | Male | Lung Adenocarcinoma | T2aN3M1b | IV | ✓ | | ELISA |
| 1043 | 60 | Male | Lung Adenocarcinoma | T2bN2M1b | IV | ✓ | ✓ | ELISA |
| 1044 | 61 | Male | Lung Adenocarcinoma | T4N3M1b | IV | ✓ | | ELISA |
| 1045 | 43 | Male | Lung Adenocarcinoma | T4N3M1b | IV | | | ELISA |
| 1046 | 62 | Female | Lung Adenocarcinoma | T3N3M1b | IV | | | ELISA |
| 1047 | 58 | Male | Lung Adenocarcinoma | T4N3M1c | IV | ✓ | ✓ | ELISA |
| 1048 | 52 | Male | Lung Adenocarcinoma | T2bN1M1a | IV | ✓ | ✓ | ELISA |
| 1049 | 64 | Male | Lung Adenocarcinoma | T2aN3M1a | IV | ✓ | | ELISA |
| 1050 | 52 | Female | Lung Adenocarcinoma | T2bN2M1c | IV | | | ELISA |
| 1051 | 52 | Female | Lung Adenocarcinoma | T4N2M1b | IV | | | ELISA |
| 1052 | 41 | Female | Lung Adenocarcinoma | T4N1M1c | IV | | | ELISA |
| 1053 | 71 | Female | Lung Adenocarcinoma | T3N3M1b | IV | ✓ | | ELISA |
| 1054 | 62 | Female | Lung Adenocarcinoma | T2bN2M1a | IV | ✓ | ✓ | ELISA |
| 1055 | 66 | Male | Lung Adenocarcinoma | T4N2M1c | IV | | | ELISA |
| 1056 | 71 | Female | Lung Adenocarcinoma | T4N3M1c | IV | | | ELISA |
| 1057 | 57 | Male | Squamous Cell LC | T1aN0M0 | Ia | ✓ | ✓ | ELISA |
| 1058 | 76 | Male | Squamous Cell LC | T1aN0M0 | Ia | ✓ | | ELISA |
| 1059 | 59 | Male | Squamous Cell LC | T1bN0M0 | Ia | ✓ | | ELISA |
| 1060 | 68 | Male | Squamous Cell LC | T2N0M0 | Ib | ✓ | | ELISA |
| 1061 | 59 | Male | Squamous Cell LC | T1aN0M0 | Ia | ✓ | | ELISA |
| 1062 | 62 | Male | Squamous Cell LC | T1aN0M0 | Ia | | | ELISA |
| 1063 | 72 | Male | Squamous Cell LC | T1aN0M0 | Ia | | | ELISA |
| 1064 | 69 | Female | Squamous Cell LC | T2aN0M0 | Ib | ✓ | | ELISA |
| 1065 | 57 | Male | Squamous Cell LC | T2aN0M0 | Ib | ✓ | ✓ | ELISA |
| 1066 | 65 | Male | Squamous Cell LC | T2aN0M0 | Ib | ✓ | | ELISA |
| 1067 | 54 | Female | Squamous Cell LC | T2aN0M0 | Ib | ✓ | | ELISA |
| 1068 | 48 | Female | Squamous Cell LC | T2aN0M0 | Ib | | | ELISA |
| 1069 | 74 | Male | Squamous Cell LC | T2bN0M0 | IIa | ✓ | | ELISA |
| 1070 | 65 | Male | Squamous Cell LC | T2bN0M0 | IIa | ✓ | | ELISA |
| 1071 | 58 | Female | Squamous Cell LC | T2bN0M0 | IIa | | | ELISA |
| 1072 | 70 | Male | Squamous Cell LC | T3N0M0 | IIb | ✓ | | ELISA |
| 1073 | 58 | Male | Squamous Cell LC | T3N0M0 | IIb | ✓ | ✓ | ELISA |
| 1074 | 76 | Male | Squamous Cell LC | T2aN1M0 | IIb | | | ELISA |
| 1075 | 67 | Male | Squamous Cell LC | T1aN1M0 | IIb | ✓ | | ELISA |
| 1076 | 68 | Male | Squamous Cell LC | T2aN1M0 | IIb | ✓ | | ELISA |
| 1077 | 70 | Male | Squamous Cell LC | T4N1M0 | IIIa | | | ELISA |
| 1078 | 65 | Male | Squamous Cell LC | T1aN2M0 | IIIa | ✓ | | ELISA |
| 1079 | 58 | Male | Squamous Cell LC | T1CN2M0 | IIIa | ✓ | ✓ | ELISA |
| 1080 | 60 | Male | Squamous Cell LC | T4N2M0 | IIIb | ✓ | | ELISA |
| 1081 | 72 | Female | Squamous Cell LC | T1bN3M0 | IIIb | ✓ | | ELISA |
| 1082 | 61 | Male | Squamous Cell LC | T2aN3M0 | IIIb | ✓ | ✓ | ELISA |
| 1083 | 47 | Male | Squamous Cell LC | T3N1M0 | IIIa | ✓ | ✓ | ELISA |
| 1084 | 54 | Male | Squamous Cell LC | T4N1M0 | IIIa | | | ELISA |
| 1085 | 64 | Male | Squamous Cell LC | T4N1M0 | IIIa | ✓ | | ELISA |
| 1086 | 73 | Male | Squamous Cell LC | T1bN2M0 | IIIa | ✓ | | ELISA |
| 1087 | 64 | Male | Squamous Cell LC | T4N2M0 | IIIb | ✓ | | ELISA |
| 1088 | 62 | Male | Squamous Cell LC | T3N2M0 | IIIb | ✓ | ✓ | ELISA |
| 1089 | 70 | Female | Squamous Cell LC | T2aN3M0 | IIIb | | | ELISA |
| 1090 | 54 | Male | Squamous Cell LC | T3N1M0 | IIIa | ✓ | | ELISA |
| 1091 | 51 | Male | Squamous Cell LC | T4N2M0 | IIIb | ✓ | ✓ | ELISA |
| 1092 | 57 | Male | Squamous Cell LC | T1bN3M0 | IIIb | ✓ | ✓ | ELISA |
| 1093 | 66 | Male | Squamous Cell LC | T2aN3M0 | IIIb | ✓ | | ELISA |
| 1094 | 61 | Female | Squamous Cell LC | T3N1M0 | IIIa | | | ELISA |
| 1095 | 54 | Male | Squamous Cell LC | T3N2M1b | IV | ✓ | ✓ | ELISA |
| 1096 | 76 | Female | Squamous Cell LC | T4N2M1c | IV | ✓ | | ELISA |
| 1097 | 51 | Male | Squamous Cell LC | T4N3M1a | IV | ✓ | ✓ | ELISA |
| 1098 | 69 | Male | Squamous Cell LC | T4N2M1c | IV | ✓ | | ELISA |
| 1099 | 72 | Male | Squamous Cell LC | T3N2M1c | IV | | | ELISA |
| 1100 | 64 | Male | Squamous Cell LC | T4N2M1b | IV | | | ELISA |
| 1101 | 86 | Male | Squamous Cell LC | T4N3M1c | IV | | | ELISA |

TABLE 2
Candidate Proteins identified from HuProt Array

| | | | Lung | | Large cell | Adenocarcinoma |
|---|---|---|---|---|---|---|
| Protein | Channel | ID | cancer^{a} | SCLC^{b} | LC^{c} | LC^{d} |
| YWHAZ | Anti-IgG | JHU00095.P001A01 | 0.631 | 0.675 | 0.810 | 0.592 |
| ARF6 | Anti-IgG | JHU00197.P003A02 | 0.638 | 0.700 | 0.771 | 0.633 |
| PDXK | Anti-IgG | JHU00349.P004A01 | 0.625 | 0.700 | 0.771 | 0.592 |
| DBN1 | Anti-IgG | JHU00310.P004A02 | 0.613 | 0.675 | 0.760 | 0.625 |
| CHIC2 | Anti-IgG | JHU03864.P041B02 | 0.638 | 0.750 | 0.538 | 0.563 |
| HK2 | Anti-IgG | JHU00137.P002A04 | 0.631 | 0.700 | 0.746 | 0.650 |
| CDK9 | Anti-IgG | JHU00114.P002B03 | 0.606 | 0.650 | 0.735 | 0.621 |
| BRSK2 | Anti-IgG | JHU00107.P002B04 | 0.606 | 0.650 | 0.735 | 0.621 |
| PDIK1L | Anti-IgG | JHU00348.P004A04 | 0.606 | 0.650 | 0.735 | 0.621 |
| FAM104B | Anti-IgG | JHU12283.P129G01 | 0.625 | 0.550 | 0.615 | 0.729 |
| RAC3 | Anti-IgG | JHU03240.P034G01 | 0.606 | 0.725 | 0.513 | 0.496 |
| RND3 | Anti-IgG | JHU14238.P149H03 | 0.606 | 0.725 | 0.538 | 0.521 |
| CCDC9 | Anti-IgG | JHU08753.P203A01 | 0.625 | 0.550 | 0.565 | 0.721 |
| ATP12A | Anti-IgG | JHU08360.P203A10 | 0.619 | 0.575 | 0.540 | 0.717 |
| YARS | Anti-IgG | JHU18237.P209G01 | 0.600 | 0.500 | 0.502 | 0.717 |
| NFE2 | Anti-IgG | JHU02166.P024G02 | 0.613 | 0.575 | 0.519 | 0.708 |
| RBPJ | Anti-IgG | JHU11111.P116E01 | 0.613 | 0.650 | 0.708 | 0.629 |
| SGK1 | Anti-IgG | JHU00076.P001A02 | 0.606 | 0.675 | 0.708 | 0.588 |
| HRAS | Anti-IgG | JHU13735.P144A09 | 0.613 | 0.700 | 0.515 | 0.567 |
| FAM172A | Anti-IgG | JHU00887.P010C04 | 0.594 | 0.550 | 0.540 | 0.696 |
| MDH2 | Anti-IgG | JHU13830.P145A01 | 0.588 | 0.550 | 0.515 | 0.692 |
| PPCDC | Anti-IgG | JHU00834.P009A05 | 0.581 | 0.475 | 0.506 | 0.692 |
| RAP1GDS1 | Anti-IgG | JHU00357.P004E06 | 0.625 | 0.600 | 0.577 | 0.688 |
| SNX9 | Anti-IgG | JHU14342.P150C02 | 0.656 | 0.675 | 0.552 | 0.683 |
| MED8 | Anti-IgG | JHU01101.P012B03 | 0.594 | 0.550 | 0.552 | 0.683 |
| KJ902876 | Anti-IgG | JHU01132.P012C02 | 0.581 | 0.550 | 0.475 | 0.683 |
| TGIF2LY | Anti-IgG | JHU16776.P207A11 | 0.600 | 0.550 | 0.565 | 0.679 |
| RBPJ | Anti-IgG | JHU09770.P102D12 | 0.613 | 0.675 | 0.673 | 0.583 |
| SEPHS1 | Anti-IgG | JHU06506.P068H06 | 0.606 | 0.675 | 0.577 | 0.500 |
| VCP | Anti-IgG | JHU15087.P158G12 | 0.594 | 0.675 | 0.500 | 0.583 |
| RBPJ | Anti-IgG | JHU13657.P143C01 | 0.594 | 0.675 | 0.660 | 0.588 |
| GSTM3 | Anti-IgG | JHU02243.P024B10 | 0.588 | 0.675 | 0.500 | 0.542 |
| NINJ1 | Anti-IgG | JHU02068.P022C09 | 0.550 | 0.675 | 0.438 | 0.463 |
| COL5A2 | Anti-IgG | JHU15971.P168H10 | 0.625 | 0.675 | 0.596 | 0.667 |
| CLEC7A | Anti-IgG | JHU03283.P035G10 | 0.600 | 0.675 | 0.500 | 0.604 |
| UBL3 | Anti-IgG | JHU16242.P170C06 | 0.600 | 0.675 | 0.538 | 0.542 |
| CSNK2B | Anti-IgG | JHU10864.P188B11 | 0.594 | 0.675 | 0.502 | 0.529 |
| TPK1 | Anti-IgG | JHU13974.P179C12 | 0.594 | 0.675 | 0.538 | 0.542 |
| RHOG | Anti-IgG | JHU13771.P144H07 | 0.594 | 0.675 | 0.500 | 0.521 |
| C6orf141 | Anti-IgG | JHU06448.P068G09 | 0.581 | 0.675 | 0.500 | 0.500 |
| PMAIP1 | Anti-IgG | JHU10143.P106D05 | 0.569 | 0.675 | 0.488 | 0.492 |
| TPK1 | Anti-IgG | JHU13974.P180C12 | 0.594 | 0.675 | 0.488 | 0.575 |
| NID2 | Anti-IgG | JHU07642.P077H04 | 0.588 | 0.675 | 0.513 | 0.496 |
| CYSRT1 | Anti-IgG | JHU06277.P066D07 | 0.569 | 0.675 | 0.513 | 0.475 |
| LRBA | Anti-IgG | JHU09554.P100E02 | 0.569 | 0.675 | 0.513 | 0.517 |
| DIRAS1 | Anti-IgG | JHU03096.P033G08 | 0.569 | 0.675 | 0.513 | 0.496 |
| MFSD2A | Anti-IgG | JHU00820.P009D04 | 0.569 | 0.475 | 0.452 | 0.667 |
| TEX264 | Anti-IgG | JHU02290.P024E03 | 0.606 | 0.625 | 0.375 | 0.667 |
| TIPIN | Anti-IgG | JHU06997.P005C07 | 0.581 | 0.525 | 0.542 | 0.663 |
| ACER2 | Anti-IgG | JHU15962.P168C12 | 0.588 | 0.600 | 0.662 | 0.638 |
| RILPL1 | Anti-IgG | JHU15985.P168G10 | 0.563 | 0.500 | 0.527 | 0.658 |
| KCNAB2 | Anti-IgG | JHU07150.P075E02 | 0.581 | 0.550 | 0.488 | 0.658 |
| CLDN2 | Anti-IgG | JHU00792.P009C04 | 0.588 | 0.575 | 0.440 | 0.658 |
| NAPG | Anti-IgG | JHU02258.P024B12 | 0.556 | 0.550 | 0.542 | 0.475 |
| FBXO2 | Anti-IgG | JHU08293.P087H05 | 0.556 | 0.550 | 0.504 | 0.496 |
| GSTA3 | Anti-IgG | JHU08773.P092E09 | 0.575 | 0.650 | 0.538 | 0.521 |
| EDIL3 | Anti-IgG | JHU06744.P071E01 | 0.581 | 0.650 | 0.538 | 0.542 |
| TPD52L3 | Anti-IgG | JHU00186.P192G10 | 0.581 | 0.650 | 0.475 | 0.579 |
| NSG1 | Anti-IgG | JHU02227.P024H10 | 0.581 | 0.650 | 0.475 | 0.558 |
| TMEM185B | Anti-IgG | JHU12047.P126D08 | 0.581 | 0.650 | 0.513 | 0.496 |
| PCYT1B | Anti-IgG | JHU05629.P059H06 | 0.575 | 0.650 | 0.538 | 0.500 |
| TCEAL3 | Anti-IgG | JHU02196.P023B05 | 0.569 | 0.650 | 0.500 | 0.500 |
| CMTM6 | Anti-IgG | JHU02425.P026E06 | 0.569 | 0.650 | 0.500 | 0.500 |
| GNMT | Anti-IgG | JHU08003.P084E11 | 0.556 | 0.650 | 0.475 | 0.517 |
| ALG8 | Anti-IgG | JHU00388.P005A12 | 0.550 | 0.500 | 0.477 | 0.650 |
| XM_006718518.2 | Anti-IgG | JHU15242.P160F07 | 0.581 | 0.650 | 0.521 | 0.613 |
| DPEP2 | Anti-IgG | JHU00698.P008A06 | 0.606 | 0.600 | 0.615 | 0.646 |
| ESYT1 | Anti-IgG | JHU15315.P161B06 | 0.594 | 0.500 | 0.577 | 0.646 |
| CX3CR1 | Anti-IgG | JHU00210.P003F08 | 0.588 | 0.575 | 0.538 | 0.646 |
| KRAS | Anti-IgG | JHU00236.P003B03 | 0.575 | 0.625 | 0.644 | 0.604 |
| CTRL | Anti-IgG | JHU18993.P193H03 | 0.581 | 0.625 | 0.502 | 0.529 |
| BC089413 | Anti-IgG | JHU16001.P168H12 | 0.538 | 0.475 | 0.388 | 0.642 |
| RTN3 | Anti-IgG | JHU01122.P012A03 | 0.581 | 0.525 | 0.552 | 0.642 |
| OSER1 | Anti-IgG | JHU01070.P012H10 | 0.575 | 0.525 | 0.513 | 0.642 |
| MRPL44 | Anti-IgG | JHU00922.P010A01 | 0.569 | 0.525 | 0.475 | 0.642 |
| ZFP69B | Anti-IgG | JHU00863.P009A02 | 0.563 | 0.500 | 0.475 | 0.642 |
| DAPP1 | Anti-IgG | JHU04340.P177H10 | 0.581 | 0.625 | 0.442 | 0.563 |
| BC071804.1 | Anti-IgG | JHU15329.P161G04 | 0.569 | 0.550 | 0.450 | 0.638 |
| H1F0 | Anti-IgG | JHU00425.P005D05 | 0.563 | 0.525 | 0.463 | 0.633 |
| TP53 | Anti-IgG | JHU13973.P146F08 | 0.563 | 0.550 | 0.538 | 0.521 |
| UAP1 | Anti-IgG | JHU00762.P008A09 | 0.563 | 0.525 | 0.479 | 0.575 |
| FTL | Anti-IgG | JHU06552.P069H07 | 0.563 | 0.625 | 0.488 | 0.492 |
| MMRN2 | Anti-IgG | JHU16011.P168A12 | 0.588 | 0.625 | 0.583 | 0.608 |
| SDCBP | Anti-IgG | JHU14785.P155C01 | 0.588 | 0.625 | 0.552 | 0.621 |
| CMTM7 | Anti-IgG | JHU12747.P134D04 | 0.581 | 0.625 | 0.538 | 0.542 |
| NME4 | Anti-IgG | JHU15632.P164F02 | 0.569 | 0.625 | 0.500 | 0.563 |
| TEX12 | Anti-IgG | JHU04021.P042C02 | 0.569 | 0.625 | 0.475 | 0.579 |
| RAB28 | Anti-IgG | JHU08423.P088B01 | 0.569 | 0.625 | 0.538 | 0.500 |
| PAFAH1B3 | Anti-IgG | JHU04573.P178A07 | 0.556 | 0.625 | 0.500 | 0.500 |
| SMAP | Anti-IgG | JHU01269.P014A08 | 0.575 | 0.550 | 0.552 | 0.621 |
| DMRT2 | Anti-IgG | JHU16721.P207E01 | 0.575 | 0.550 | 0.508 | 0.596 |
| CD200 | Anti-IgG | JHU00499.P006B09 | 0.575 | 0.525 | 0.540 | 0.613 |
| APOH | Anti-IgG | JHU04901.P035B02 | 0.575 | 0.600 | 0.463 | 0.613 |
| FIGNL2 | Anti-IgG | JHU17712.P220G05 | 0.556 | 0.550 | 0.427 | 0.579 |
| CTAG1A | Anti-IgG | JHU17795.P221D02 | 0.544 | 0.500 | 0.500 | 0.542 |
| BC073794.1 | Anti-IgG | JHU16436.P173E01 | 0.531 | 0.500 | 0.400 | 0.608 |
| IGHA1 | Anti-IgG | JHU16422.P173F01 | 0.525 | 0.500 | 0.400 | 0.608 |
| ETHE1 | Anti-IgG | JHU00417.P005D10 | 0.519 | 0.475 | 0.402 | 0.533 |
| NRG3 | Anti-IgG | JHU19408.P197D01 | 0.525 | 0.500 | 0.475 | 0.496 |
| USP7 | Anti-IgG | JHU17961.P222F02 | 0.556 | 0.525 | 0.538 | 0.604 |
| LIMS1 | Anti-IgG | JHU14949.P157E01 | 0.594 | 0.600 | 0.577 | 0.604 |
| GPR63 | Anti-IgG | JHU05687.P060H05 | 0.556 | 0.500 | 0.577 | 0.604 |
| STRA13 | Anti-IgG | JHU08340.P087A07 | 0.500 | 0.600 | 0.431 | 0.492 |
| POLD4 | Anti-IgG | JHU11576.P121B02 | 0.519 | 0.600 | 0.562 | 0.496 |
| CKM | Anti-IgG | JHU00018.P189A02 | 0.550 | 0.600 | 0.500 | 0.500 |
| IGLL1 | Anti-IgM | JHU00528.P006D05 | 0.631 | 0.575 | 0.554 | 0.588 |
| UFD1L | Anti-IgM | JHU02300.P024H09 | 0.663 | 0.750 | 0.565 | 0.658 |
| TADA3 | Anti-IgM | JHU00752.P008D11 | 0.619 | 0.525 | 0.658 | 0.579 |
| TMEM129 | Anti-IgM | JHU01626.P017F02 | 0.581 | 0.550 | 0.708 | 0.567 |
| GAD1 | Anti-IgM | JHU00520.P006D09 | 0.638 | 0.650 | 0.579 | 0.592 |
| WSCD1 | Anti-IgM | JHU00764.P008F08 | 0.619 | 0.550 | 0.669 | 0.567 |
| TMEM187 | Anti-IgM | JHU01135.P012B07 | 0.606 | 0.575 | 0.606 | 0.542 |
| CD200 | Anti-IgM | JHU00499.P006B09 | 0.619 | 0.550 | 0.577 | 0.625 |
| GLI4 | Anti-IgM | JHU05601.P006B10 | 0.606 | 0.525 | 0.629 | 0.579 |
| ITLN1 | Anti-IgM | JHU00619.P007D11 | 0.619 | 0.550 | 0.681 | 0.575 |
| ADPRHL2 | Anti-IgM | JHU00483.P006C10 | 0.588 | 0.550 | 0.540 | 0.550 |
| SLAMF6 | Anti-IgM | JHU16030.P168H09 | 0.613 | 0.500 | 0.667 | 0.683 |
| C1QTNF1 | Anti-IgM | JHU00492.P005D06 | 0.619 | 0.600 | 0.554 | 0.608 |
| TGIF2 | Anti-IgM | JHU00754.P008D12 | 0.600 | 0.525 | 0.631 | 0.567 |
| KJ902876 | Anti-IgM | JHU01132.P012C02 | 0.625 | 0.625 | 0.642 | 0.679 |
| PPCDC | Anti-IgM | JHU00834.P009A05 | 0.650 | 0.650 | 0.617 | 0.675 |
| CASC4 | Anti-IgM | JHU00788.P009D02 | 0.631 | 0.675 | 0.579 | 0.654 |
| PARP11 | Anti-IgM | JHU01887.P020F10 | 0.638 | 0.675 | 0.513 | 0.663 |
| VPREB1 | Anti-IgM | JHU00668.P007C12 | 0.575 | 0.525 | 0.594 | 0.513 |
| LGALS9 | Anti-IgM | JHU19305.P196F12 | 0.619 | 0.600 | 0.552 | 0.621 |
| TIPIN | Anti-IgM | JHU06997.P005C07 | 0.619 | 0.550 | 0.642 | 0.617 |
| ORMDL2 | Anti-IgM | JHU00928.P010A11 | 0.600 | 0.550 | 0.604 | 0.575 |
| YARS | Anti-IgM | JHU18237.P209G01 | 0.594 | 0.525 | 0.660 | 0.629 |
| KRCC1 | Anti-IgM | JHU01192.P013A01 | 0.588 | 0.550 | 0.604 | 0.658 |
| APEX1 | Anti-IgM | JHU00391.P005C08 | 0.600 | 0.550 | 0.656 | 0.571 |
| SDCBP | Anti-IgM | JHU14785.P155C01 | 0.569 | 0.525 | 0.654 | 0.583 |
| MRPL32 | Anti-IgM | JHU01018.P011C11 | 0.600 | 0.650 | 0.552 | 0.579 |
| SENP3 | Anti-IgM | JHU07173.P075A10 | 0.569 | 0.650 | 0.590 | 0.558 |
| HRH1 | Anti-IgM | JHU07143.P075A07 | 0.563 | 0.650 | 0.538 | 0.521 |
| BEST1 | Anti-IgM | JHU07113.P075A11 | 0.550 | 0.650 | 0.552 | 0.517 |
| LGALS8 | Anti-IgM | JHU15337.P161H11 | 0.606 | 0.650 | 0.604 | 0.554 |
| CLCF1 | Anti-IgM | JHU00895.P010D07 | 0.594 | 0.650 | 0.527 | 0.596 |
| FOXRED1 | Anti-IgM | JHU00706.P008C08 | 0.600 | 0.525 | 0.629 | 0.600 |
| SP110 | Anti-IgM | JHU00660.P007A09 | 0.594 | 0.550 | 0.552 | 0.600 |
| HYLS1 | Anti-IgM | JHU01186.P013A02 | 0.594 | 0.575 | 0.577 | 0.646 |
| MMRN2 | Anti-IgM | JHU16011.P168A12 | 0.569 | 0.525 | 0.538 | 0.646 |
| BAG1 | Anti-IgM | JHU01064.P012F03 | 0.531 | 0.500 | 0.633 | 0.513 |
| ZFYVE21 | Anti-IgM | JHU00480.P005C12 | 0.550 | 0.475 | 0.633 | 0.554 |
| ELAC1 | Anti-IgM | JHU00699.P008B12 | 0.581 | 0.525 | 0.581 | 0.579 |
| ZNF330 | Anti-IgM | JHU02976.P031B03 | 0.563 | 0.525 | 0.538 | 0.542 |
| H1F0 | Anti-IgM | JHU00425.P005D05 | 0.581 | 0.550 | 0.475 | 0.621 |
| DPP4 | Anti-IgM | JHU00902.P010B11 | 0.606 | 0.600 | 0.577 | 0.625 |
| ARHGAP1 | Anti-IgM | JHU18430.P212E10 | 0.588 | 0.625 | 0.615 | 0.583 |
| BC089414.1 | Anti-IgM | JHU16000.P168G09 | 0.600 | 0.600 | 0.577 | 0.625 |
| ZNF280B | Anti-IgM | JHU08043.P084C03 | 0.588 | 0.625 | 0.565 | 0.575 |
| TCP11L2 | Anti-IgM | JHU08048.P084C08 | 0.581 | 0.625 | 0.577 | 0.542 |
| CSNK1E | Anti-IgM | JHU04057.P024H04 | 0.581 | 0.625 | 0.538 | 0.563 |
| NUDT11 | Anti-IgM | JHU17366.P208E10 | 0.581 | 0.625 | 0.565 | 0.533 |
| FSCN1 | Anti-IgM | JHU14110.P148G03 | 0.581 | 0.625 | 0.552 | 0.558 |
| MAPK10 | Anti-IgM | JHU07156.P075A05 | 0.575 | 0.625 | 0.552 | 0.579 |
| GLT1D1 | Anti-IgM | JHU08002.P084C04 | 0.575 | 0.625 | 0.577 | 0.563 |
| ZDHHC7 | Anti-IgM | JHU08060.P084C07 | 0.575 | 0.625 | 0.590 | 0.558 |
| CELA3A | Anti-IgM | JHU01285.P014E09 | 0.575 | 0.625 | 0.577 | 0.563 |
| SLC25A45 | Anti-IgM | JHU07373.P080D04 | 0.569 | 0.625 | 0.577 | 0.563 |
| PHF10 | Anti-IgM | JHU01109.P012B06 | 0.563 | 0.500 | 0.500 | 0.625 |
| TMEM38B | Anti-IgM | JHU02293.P024E09 | 0.525 | 0.625 | 0.404 | 0.521 |
| C6orf203 | Anti-IgM | JHU00888.P010D09 | 0.581 | 0.575 | 0.565 | 0.554 |
| CLDN1 | Anti-IgM | JHU00504.P006C05 | 0.594 | 0.550 | 0.590 | 0.621 |
| GAPDH | Anti-IgM | JHU00420.P005A10 | 0.569 | 0.500 | 0.552 | 0.621 |
| SS18 | Anti-IgM | JHU16035.P168B01 | 0.563 | 0.600 | 0.456 | 0.621 |
| ABT1 | Anti-IgM | JHU00578.P007G05 | 0.538 | 0.500 | 0.540 | 0.488 |
| C1R | Anti-IgM | JHU07976.P084D01 | 0.581 | 0.600 | 0.615 | 0.583 |
| GPR63 | Anti-IgM | JHU05687.P060H05 | 0.569 | 0.550 | 0.615 | 0.583 |
| ZFP69B | Anti-IgM | JHU00863.P009A02 | 0.594 | 0.600 | 0.540 | 0.613 |
| MRPL44 | Anti-IgM | JHU00922.P010A01 | 0.575 | 0.550 | 0.540 | 0.613 |
| CDH6 | Anti-IgM | JHU02221.P024F09 | 0.581 | 0.600 | 0.538 | 0.563 |
| CD96 | Anti-IgM | JHU00591.P007A10 | 0.563 | 0.525 | 0.500 | 0.583 |
| NFE2 | Anti-IgM | JHU02166.P024G02 | 0.569 | 0.550 | 0.577 | 0.604 |
| LSM14A | Anti-IgM | JHU01013.P011A08 | 0.569 | 0.525 | 0.538 | 0.604 |
| DAZ2 | Anti-IgM | JHU19714.P183E10 | 0.563 | 0.525 | 0.538 | 0.604 |
| TBC1D23 | Anti-IgM | JHU01133.P012A07 | 0.563 | 0.550 | 0.500 | 0.604 |
| MAK16 | Anti-IgM | JHU08034.P084E04 | 0.575 | 0.575 | 0.604 | 0.575 |
| SULF2 | Anti-IgM | JHU01128.P012D07 | 0.575 | 0.550 | 0.604 | 0.575 |
| RECQL5 | Anti-IgM | JHU00938.P010B05 | 0.594 | 0.600 | 0.565 | 0.596 |
| KLRC4 | Anti-IgM | JHU00621.P007C09 | 0.569 | 0.600 | 0.527 | 0.533 |
| ZNF226 | Anti-IgM | JHU00672.P007E11 | 0.525 | 0.600 | 0.585 | 0.492 |
| CYP46A1 | Anti-IgM | JHU00795.P009A09 | 0.575 | 0.600 | 0.577 | 0.563 |

^{a}Discriminative ability for detecting lung cancer vs. a healthy subject
^{b}Discriminative ability for detecting small cell lung cancer (SCLC) vs. a healthy subject
^{c}Discriminative ability for detecting large cell lung cancer vs. a healthy subject
^{d}Discriminative ability for detecting pulmonary adenocarcinoma vs. a healthy subject

### Example 2. HuProt Array Serum Profiling Assay

100 serum samples, collected from 80 patients with LC and 20 healthy individuals, were individually profiled on HuProt arrays. HuProt arrays were provided by CDI Laboratories, Inc. Each HuProt v3.0 array is comprised of 20,240 unique human full-length proteins, covering ˜80% of the human proteome. LC and healthy groups did not show any significant differences in age, gender or smoking history composition (Table 3).

TABLE 3
Characteristics of the samples in Phase I.

| | LC (n = 80) | | | Healthy (n = 20) | | | |
|---|---|---|---|---|---|---|---|
| Variable | No. | Mean | % | No. | Mean | % | P |
| Age (years) | | | | | | | 0.086 |
| Mean | | 60.4 | | | 56.4 | | |
| Standard deviation | | 8.5 | | | 11.1 | | |
| Sex | | | | | | | 0.223 |
| Male | 66 | | 82.5 | 14 | | 70.0 | |
| Female | 14 | | 17.5 | 6 | | 30.0 | |

Type

| | | |
|---|---|---|
| Small Cell Lung | 20 | 25.0 |
| Cancer | | |
| Large Cell Lung | 13 | 16.3 |
| Cancer | | |
| Adenocarcinoma | 24 | 30.0 |
| Squamous Cell | 23 | 28.8 |
| Carcinoma | | |

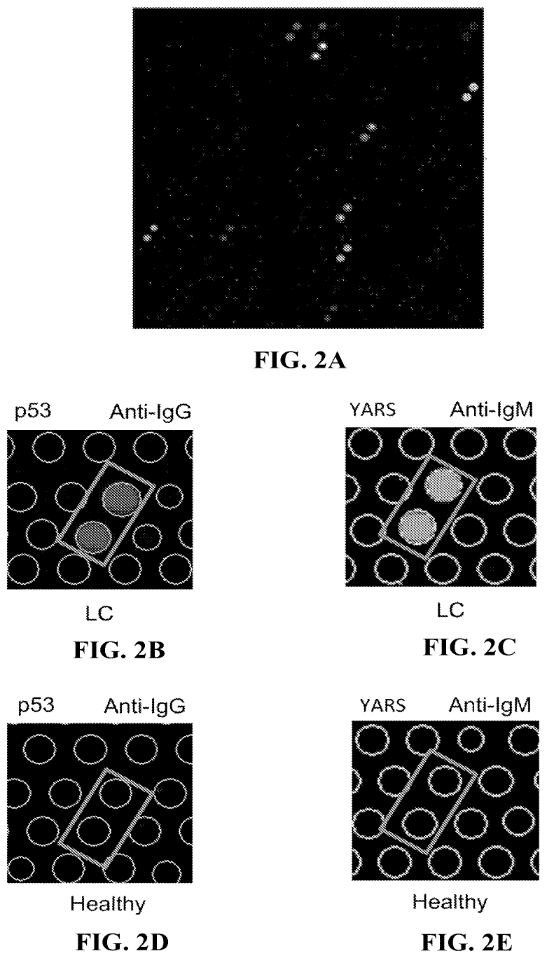

A HuProt array was first incubated with a blocking buffer (3% BSA in PBS with 0.1% (v/v) Tween 20) at room temperature (RT) for 1 hr. After a brief rinsing step, 200 μL of 1:1,000-diluted serum sample was added and incubated under a glass coverslip (LifterSlip) at RT for 1 hr. After three 10-min washes in 1×TBST with gentle shaking, the array was incubated with 200 μL of 1,000-fold diluted Alexa647-labeled goat anti-human IgG and Alexa555-labeled goat anti-human IgM antibodies (Jackson Immunes, USA) in the dark at RT for 1 hr. Subsequently, the HuProt array was washed three times with 1×TBST and then rinsed three times with double-distilled H2O. The HuProt array was spun to dryness, scanned with the Genepix 4000B Microarray Scanner, and antibody signals were acquired using GenePix Pro 6.0 software (Molecular Devices, USA). A representative scanning image is shown in FIG. 2A.

To select candidate proteins, the median values of the foreground (Fij) and background (Bij) intensity at a given protein spot (i,j) on the protein array were extracted. The signal intensity (Rij) of each protein spot was defined as Fij/Bij. Since each protein is printed in duplicate on an array, Rij was averaged for each protein as Rp. For HuProt array analysis, a Z-score (Zₚ) of each binding assay with a serum sample was calculated based on the distribution of Rp: Zₚ═(Rₚ−N)/SD, where SD and N represent the standard deviation of the distribution of Rₚ and mean of Rₚ, respectively. Using a stringent cutoff (Z≥7) positive proteins were selected. For example, p53 (FIG. 2B) and YARS (FIG. 2C) showed strong anti-human IgG and IgM signals, respectively, in LC patients, but less so in healthy subjects (FIG. 2D and FIG. 2E).

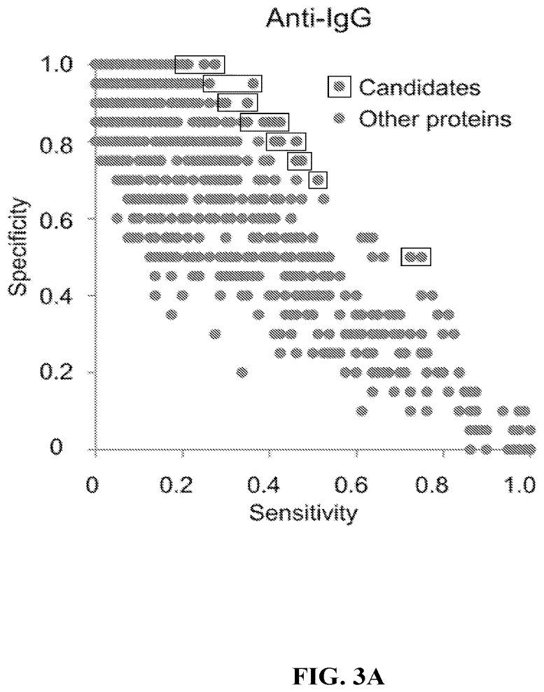

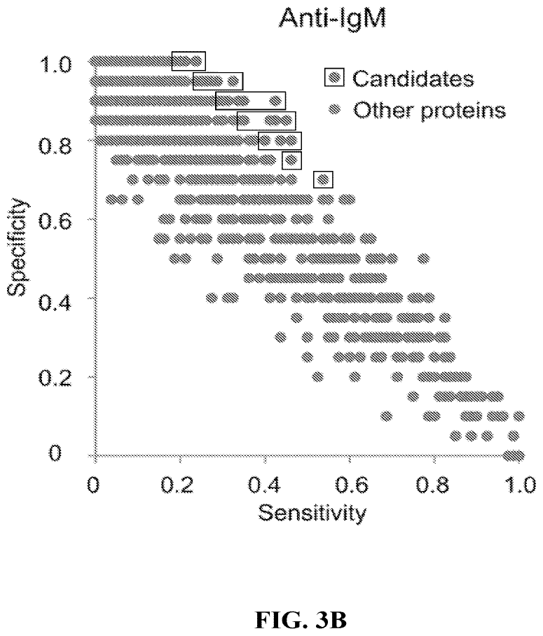

The sensitivity and specificity were calculated for each protein using the following formulae: Sensitivity=TP/(TP+FN) and Specificity=TN/(FP+TN), where the true negatives (TN) are the healthy persons correctly classified as negatives. The true positives (TP) refer to LC patients correctly classified as positives. The false negatives (FN) are LC patients incorrectly classified as negatives, and the false positives (FP) are healthy persons incorrectly classified as positives. For each comparison (LC vs negative controls), the biomarker candidates were selected with the highest discriminant ability, which is defined as: discriminant ability=(sensitivity+specificity)/2. Proteins with a discriminant ability ≥60% were selected for further validation. As a result, 170 candidate proteins were identified (Table 2), 105 and 77 of which were chosen in the anti-IgG (FIG. 3A) and -IgM (FIG. 3B) profiling, respectively. Functional enrichment analysis using DAVID identified five enriched terms, namely extracellular exosome, identical protein binding, small GTPase mediated signal transduction, protein N-terminus binding and sphingolipid signaling pathway (FDR<0.5). Six proteins were enriched in sphingolipid signaling pathway, which is known to be associated with lung cancer.

### Example 3. Construction of LC Focused Arrays and Discovery-Stage Serum Profiling Assays

To fabricate LC focused arrays, the 170 candidate proteins identified in the HuProt array experiments were printed on SuperEpoxy 2 (Arraylt, USA) slides to form 14 identical subarrays per slide. The printed LC focused arrays were stored at 4° C. under vacuum until use.

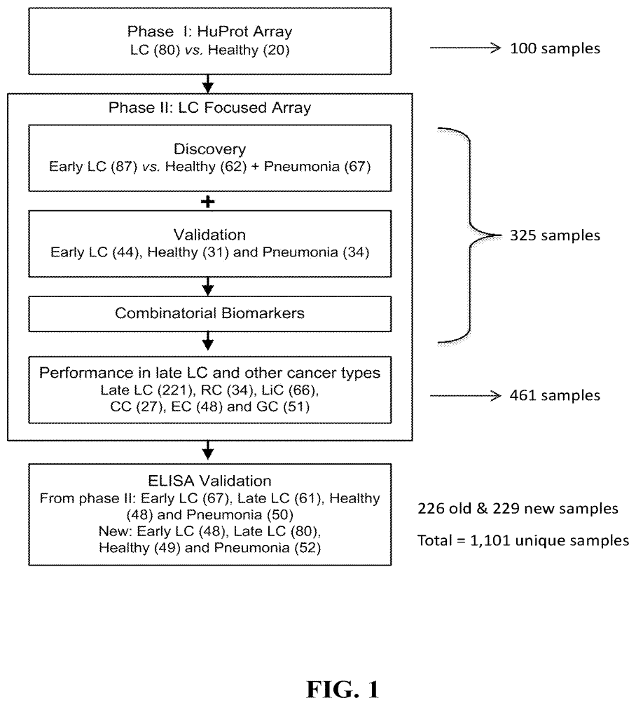

A new cohort was assembled with serum samples collected from 131 patients with early stage LC (30 limited stage SCLC, 55 stage I/II adenocarcinoma, and 46 stage I/II squamous-cell carcinoma), and 93 healthy subjects, as well as 101 serum samples from LBL patients including 83 pneumonia, 39 COPD and 31 pulmonary TB, as additional negative controls. Statistical analysis did not find any significant difference in age, gender or smoking history between the LC groups and the healthy and LBL groups combined (Table 4)). Each of LC subgroups and negative controls (healthy+LBL) were randomly split in a 2:1 ratio—two thirds were used for discovery and one third for independent validation of biomarker candidates. The discovery cohort thus included 87 early LC, 67 pnuemonia, and 62 healthy serum samples; the validation cohort included 44 early LC, 34 pnuemonia, and 31 healthy serum samples (FIG. 1).

TABLE 4
Characteristics of the samples in Phase II.

| | Early LC | | | Healthy | | | Pneumonia | | | |
|---|---|---|---|---|---|---|---|---|---|---|
| | (n = 131) | | | (n = 93) | | | (n = 101) | | | |
| Variable | No. | Mean | % | No. | Mean | % | No. | Mean | % | P |
| Age (years) | | | | | | | | | | 0.165 |
| Mean | | 61.2 | | | 58.3 | | | 61.1 | | |
| Standard deviation | | 10 | | | 8.4 | | | 8.8 | | |
| Sex | | | | | | | | | | 0.102 |
| Male | 101 | | 77.1 | 64 | | 68.8 | 69 | | 68.3 | |
| Female | 30 | | 22.9 | 29 | | 31.2 | 32 | | 31.7 | |

Type

| | | |
|---|---|---|
| Small Cell | 30 | 22.9 |
| Lung Cancer | | |
| Adenocarcinoma | 55 | 42.0 |
| Squamous Cell | 46 | 35.1 |
| Carcinoma | | |

All the serum profiling assays on the LC specific array were carried out using a similar protocol as described above. Briefly, a 14-chamber rubber gasket (GraceBio Corp, USA) was mounted onto each slide to create individual chambers for the 14 identical subarrays on each slide. The subsequent assay process was identical to that described in Example 2, with an exception that the volume of buffers or serum samples was reduced to 50 μL per subarray. Again, Alexa647-labeled goat anti-human IgG and Alexa555-labeled goat anti-human IgM antibodies were used as detection reagents under the same conditions as described above. All of the slides were washed under the same conditions as the HuProt arrays. After drying, the focused arrays were scanned with the Genepix 4000B Microarray Scanner and the antibody signals were acquired using GenePix Pro 6.0 software (Molecular Devices, Sunnyvale, CA).

The signal value for each protein was normalized by dividing the median value of negative controls for each sample. P values obtained from the T test were calculated and adjusted as false discovery rates using the GenePattern platform. The optimal cutoff value for each candidate was evaluated with two criteria: 1) at least 90% of specificity and 2) the highest discriminant ability.

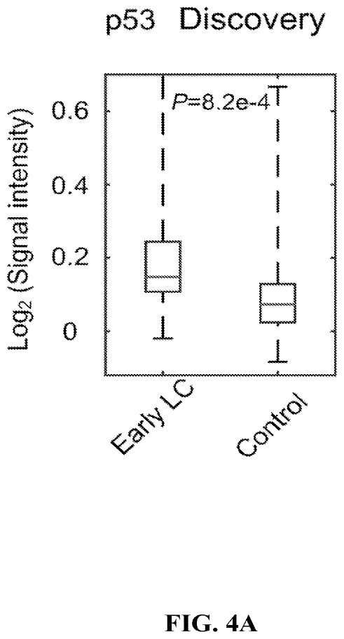

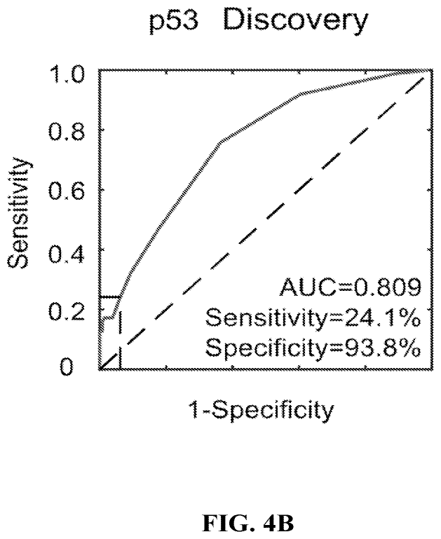

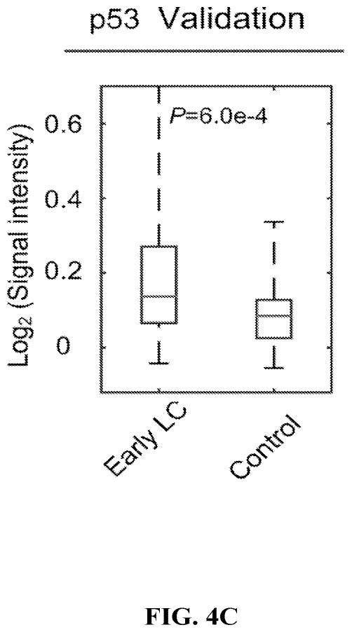

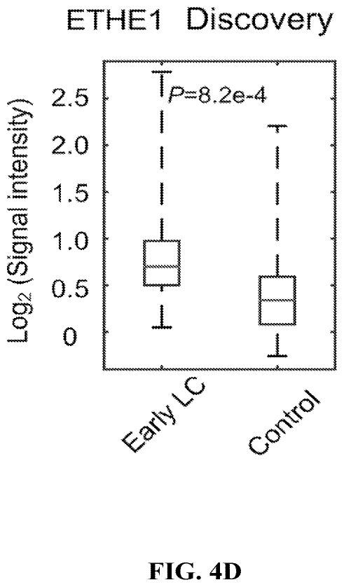

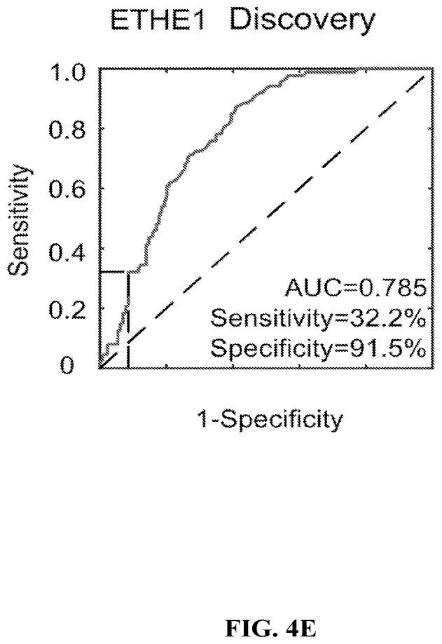

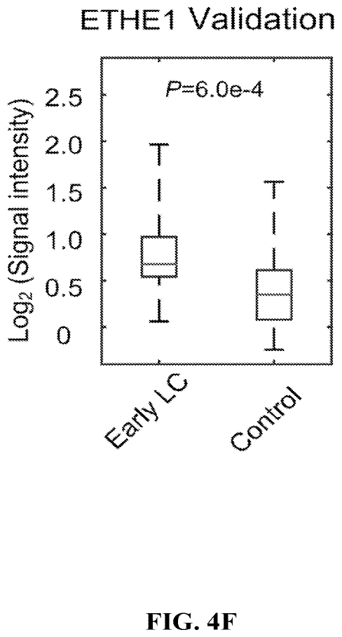

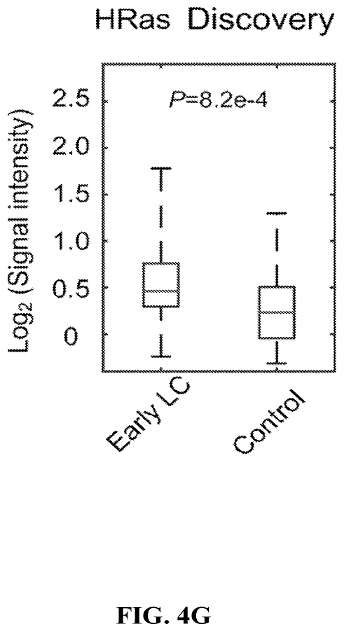

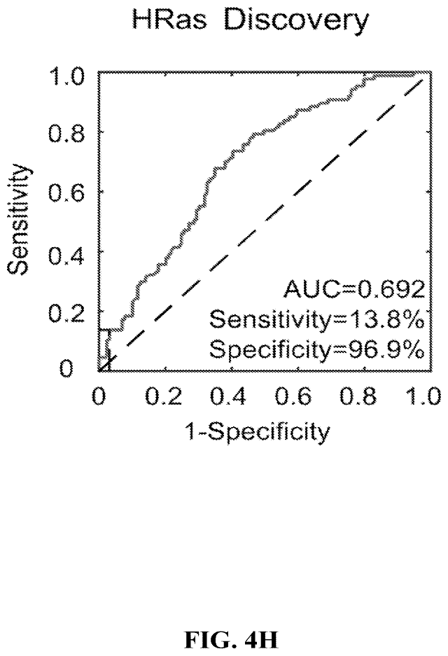

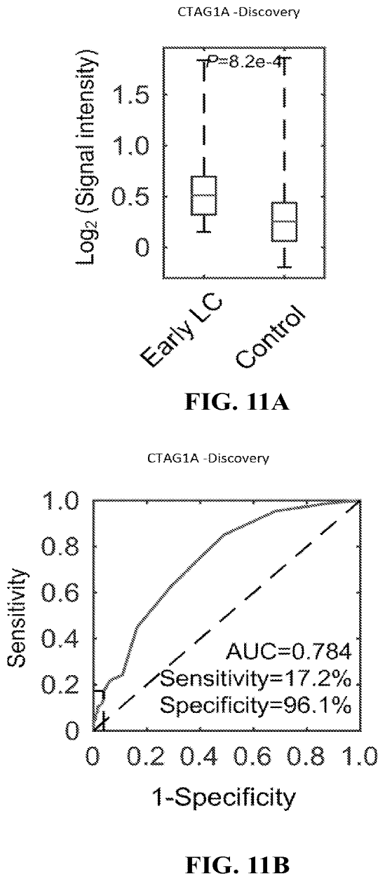

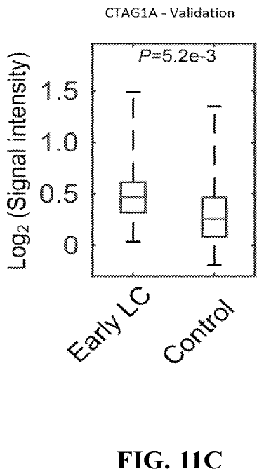

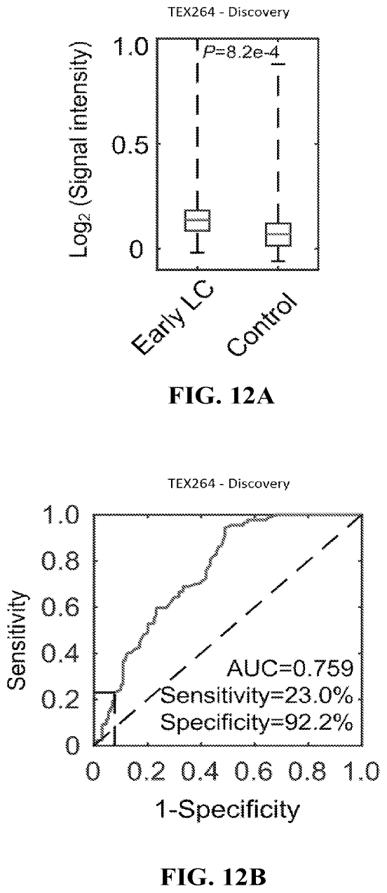

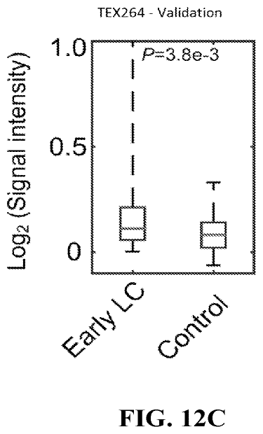

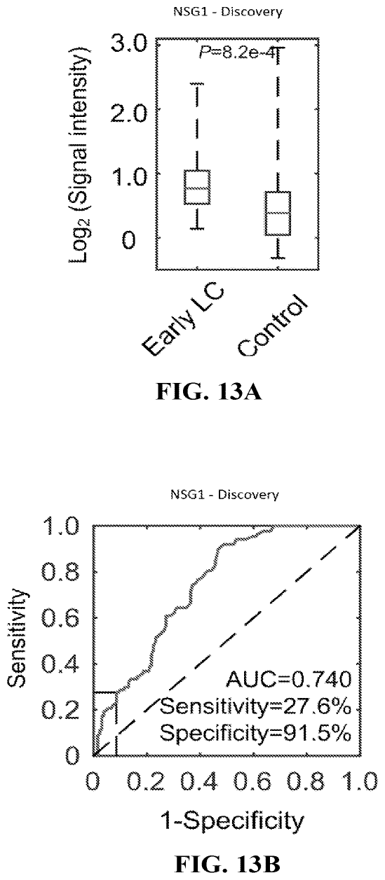

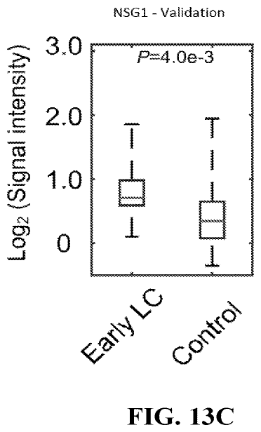

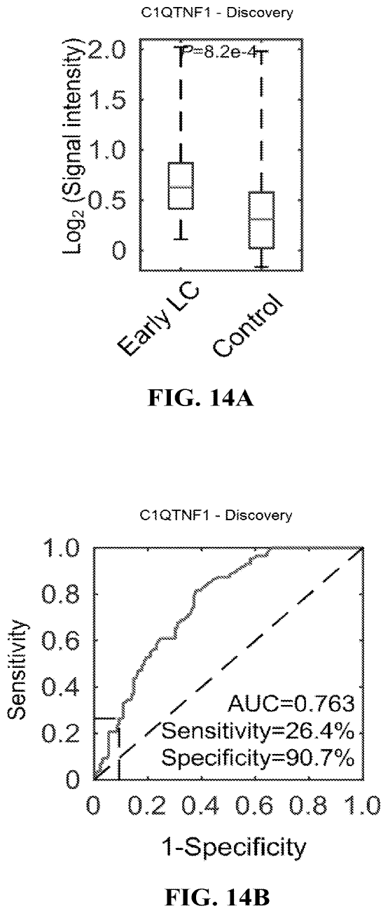

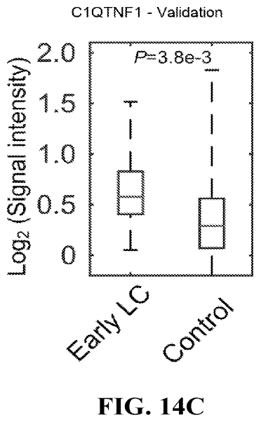

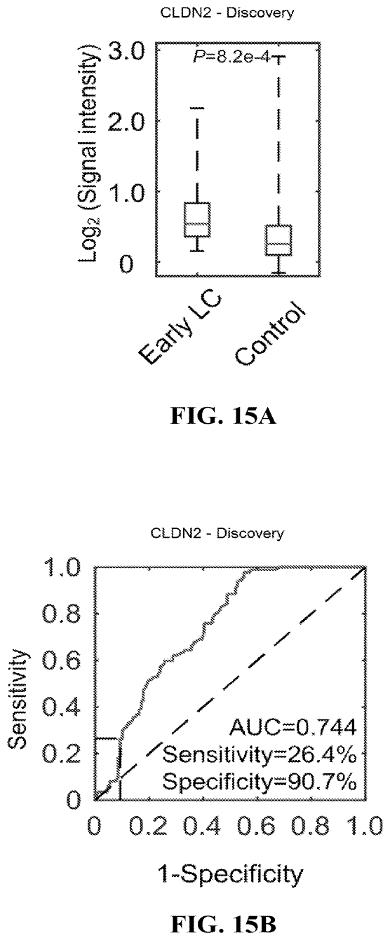

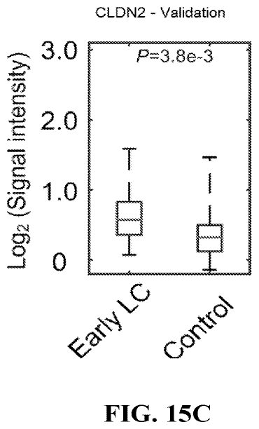

Each serum sample was profiled individually on a LC focused arrays using a similar protocol as described above. Again, both anti-IgG and -IgM profiles were obtained simultaneously for every sample. In the discovery stage, serum profiles were compared between the LC and negative control group to identify proteins that could distinguish LC from negative control group using stringent criteria—FDR<0.001 and fold change of average signal intensity between the two groups ≥1.20. This analysis resulted in identifying eight proteins, namely p53, ETHE1, CTAG1A, C1QTNF1, TEX264, CLDN2, NSG1 and HRas (Table 5). The signal distributions in the LC and negative control group were calculated for p53 (FIG. 4A), ETHE1 (FIG. 4D), HRas (FIG. 4G), CTAG1A (FIG. 11A), TEX264 (FIG. 12A), NSG1 (FIG. 13A), C1QTNF1 (FIG. 14A), and CLDN2 (FIG. 15A). To assess the performance of each candidate biomarker, areas under the ROC curves (AUCs) were calculated for p53 (FIG. 4B), ETHE1 (FIG. 4E), HRas (FIG. 4H), CTAG1A (FIG. 11B), TEX264 (FIG. 12B), NSG1 (FIG. 13B), C1QTNF1 (FIG. 14B), and CLDN2 (FIG. 15B). The ROC values of the eight proteins ranged from 0.68 to 0.81 (Table 5). Maximum discriminant ability values for each protein were calculated with a requirement of a minimum specificity of 90% to determine the optimal cut off values of signal intensity for each protein with the corresponding sensitivity and specificity values (Table 5).

TABLE 5
Performance of eight biomarkers in discovery
and validation stages of Phase II.

| | Discovery | | | | | |
|---|---|---|---|---|---|---|
| | | Cut | | | Validation | |
| Protein | AUC | off | Sensitivity | Specificity | Sensitivity | Specificity |
| P53 | 0.809 | 1.209 | 24.1% | 93.8% | 22.7% | 96.9% |
| ETHE1 | 0.785 | 1.861 | 32.2% | 91.5% | 29.5% | 93.8% |
| CTAG1A | 0.784 | 1.200 | 17.2% | 96.1% | 18.2% | 93.8% |
| C1QTNF1 | 0.763 | 1.577 | 26.4% | 90.7% | 22.7% | 93.8% |
| TEX264 | 0.759 | 2.088 | 23.0% | 92.2% | 20.5% | 93.8% |
| CLDN2 | 0.744 | 1.820 | 26.4% | 90.7% | 22.7% | 95.4% |
| NSG1 | 0.740 | 1.735 | 27.6% | 91.5% | 29.5% | 92.3% |
| HRas | 0.692 | 1.980 | 13.8% | 96.9% | 18.2% | 93.8% |

### Example 4. Validation Serum Assays

To validate these potential LC biomarkers, the signal intensity of each protein were compared between the LC and negative control group in the validation cohort. As visualized in box plots, each of p53 (FIG. 4C), ETHE1 (FIG. 4F), HRas (FIG. 4I), CTAG1A (FIG. 11C), TEX264 (FIG. 12C), NSG1 (FIG. 13C), C1QTNF1 (FIG. 14C), and CLDN2 (FIG. 15C) showed significantly higher signal intensity in the LC than the negative control group. Optimal cut off values obtained above were applied to determine the sensitivity and specificity for each protein in the validation cohort. All of the eight proteins yielded similar or better sensitivity and specificity values in the validation cohort (Table 5), confirming that the identified biomarkers have the robust classification power for early stage LC.

### Example 5. Identification of Combinatorial Biomarker Panels

To further improve the classification power of the identified biomarkers, combinatorial biomarker panels were identified and validated using the discovery and validation cohorts, respectively. To do so, performance of the eight identified biomarkers was evaluated for all possible combinations between two and eight proteins (a total of 253 combinations). A binary scoring system was first employed to convert the actual signal intensity of each protein to either 1 or 0, such that 1 represented signal intensity >the optimal cut off value, and 0 otherwise. Next the performance of every possible combination in the discovery cohort was evaluated. For a given combination of n proteins, the sum of the binary scores of the n proteins was assigned to each serum sample as a summary score. If the summary score of a sample was greater than k (1≤k≤n), the sample was called positive. The sensitivity and specificity at the best discriminant ability value were recorded for each combination. (Table 6) Finally, combinations and their k values were identified with the best discriminant ability by requiring a minimum specificity of 90%.

TABLE 6
Good combinations for early diagnosis of lung cancer

| | | | Discovery | | | | |
|---|---|---|---|---|---|---|---|
| | | Cut | | | Discriminative | Validation | |
| # | Combination | off | Sensitivity | Specificity | Ability | Sensitivity | Specificity |
| 4 | CTAG1A, NSG1, TP53, HRAS | 1 | 56.3% | 88.4% | 72.3% | 61.4% | 86.2% |
| 5 | C1QTNF1, CTAG1A, TP53, ETHE1, | 1 | 56.3% | 88.4% | 72.3% | 52.3% | 87.7% |
| | HRAS | | | | | | |
| 5 | CTAG1A, TEX264, TP53, ETHE1, | 1 | 56.3% | 88.4% | 72.3% | 54.5% | 86.2% |
| | HRAS | | | | | | |
| 4 | TEX264, TP53, ETHE1, HRAS | 1 | 55.2% | 89.1% | 72.2% | 50.0% | 89.2% |
| 4 | CTAG1A, TP53, ETHE1, HRAS | 1 | 54.0% | 89.9% | 72.0% | 52.3% | 87.7% |
| 3 | TP53, ETHE1, HRAS | 1 | 51.7% | 90.7% | 71.2% | 47.7% | 90.8% |
| 3 | NSG1, TP53, HRAS | 1 | 54.0% | 88.4% | 71.2% | 56.8% | 87.7% |
| 4 | C1QTNF1, TP53, ETHE1, HRAS | 1 | 54.0% | 88.4% | 71.2% | 47.7% | 89.2% |
| 4 | C1QTNF1, CTAG1A, TP53, ETHE1 | 1 | 50.6% | 88.4% | 69.5% | 45.5% | 89.2% |
| 4 | C1QTNF1, CTAG1A, TP53, HRAS | 1 | 50.6% | 88.4% | 69.5% | 52.3% | 89.2% |
| 4 | CLDN2, CTAG1A, TP53, HRAS | 1 | 50.6% | 88.4% | 69.5% | 54.5% | 89.2% |
| 4 | CTAG1A, TEX264, TP53, ETHE1 | 1 | 50.6% | 88.4% | 69.5% | 47.7% | 87.7% |
| 4 | CTAG1A, TEX264, TP53, HRAS | 1 | 50.6% | 88.4% | 69.5% | 50.0% | 87.7% |
| 3 | TEX264, TP53, HRAS | 1 | 49.4% | 89.1% | 69.3% | 43.2% | 90.8% |
| 3 | CTAG1A, TP53, ETHE1 | 1 | 48.3% | 89.9% | 69.1% | 45.5% | 89.2% |
| 3 | CTAG1A, NSG1, IP53 | 1 | 48.3% | 89.1% | 68.7% | 50.0% | 89.2% |
| 3 | TEX264, TP53, ETHE1 | 1 | 48.3% | 89.1% | 68.7% | 43.2% | 90.8% |
| 3 | C1QTNF1, TP53, HRAS | 1 | 48.3% | 88.4% | 68.3% | 47.7% | 90.8% |
| 3 | CLDN2, TP53, HRAS | 1 | 48.3% | 88.4% | 68.3% | 50.0% | 92.3% |
| 2 | TP53, ETHE1 | 1 | 44.8% | 90.7% | 67.8% | 40.9% | 92.3% |
| 3 | C1QTNF1, TP53, ETHE1 | 1 | 47.1% | 88.4% | 67.7% | 40.9% | 90.8% |
| 3 | CTAG1A, TP53, HRAS | 1 | 43.7% | 91.5% | 67.6% | 47.7% | 89.2% |
| 2 | NSG1, TP53 | 1 | 46.0% | 89.1% | 67.6% | 45.5% | 90.8% |
| 3 | NSG1, ETHE1, HRAS | 1 | 46.0% | 88.4% | 67.2% | 47.7% | 87.7% |
| 4 | C1QTNF1, CTAG1A, ETHE1, HRAS | 1 | 46.0% | 88.4% | 67.2% | 40.9% | 89.2% |
| 4 | CTAG1A, TEX264, ETHE1, HRAS | 1 | 46.0% | 88.4% | 67.2% | 45.5% | 87.7% |
| 3 | CTAG1A, ETHE1, HRAS | 1 | 43.7% | 89.9% | 66.8% | 40.9% | 89.2% |
| 4 | C1QTNF1, TEX264, ETHE1, HRAS | 1 | 44.8% | 88.4% | 66.6% | 40.9% | 89.2% |
| 3 | CTAG1A, NSG1, HRAS | 1 | 43.7% | 89.1% | 66.4% | 50.0% | 87.7% |
| 3 | TEX264, ETHE1, HRAS | 1 | 42.5% | 89.9% | 66.2% | 40.9% | 90.8% |
| 3 | C1QTNF1, CTAG1A, TP53 | 1 | 43.7% | 88.4% | 66.0% | 40.9% | 90.8% |
| 3 | CLDN2, CTAG1A, TP53 | 1 | 43.7% | 88.4% | 66.0% | 43.2% | 90.8% |
| 4 | C1QTNF1, CLDN2, CTAG1A, HRAS | 1 | 43.7% | 88.4% | 66.0% | 43.2% | 90.8% |
| 4 | C1QTNF1, CLDN2, TEX264, HRAS | 1 | 43.7% | 88.4% | 66.0% | 43.2% | 90.8% |
| 4 | C1QTNF1, CLDN2, ETHE1, HRAS | 1 | 43.7% | 88.4% | 66.0% | 38.6% | 90.8% |
| 4 | C1QTNF1, CTAG1A, TEX264, HRAS | 1 | 43.7% | 88.4% | 66.0% | 45.5% | 89.2% |
| 3 | C1QTNF1, ETHE1, HRAS | 1 | 41.4% | 89.1% | 65.3% | 36.4% | 90.8% |

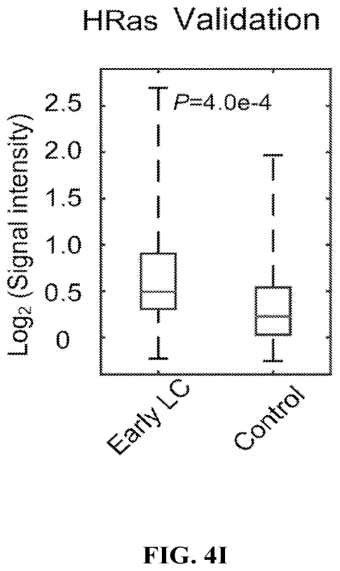

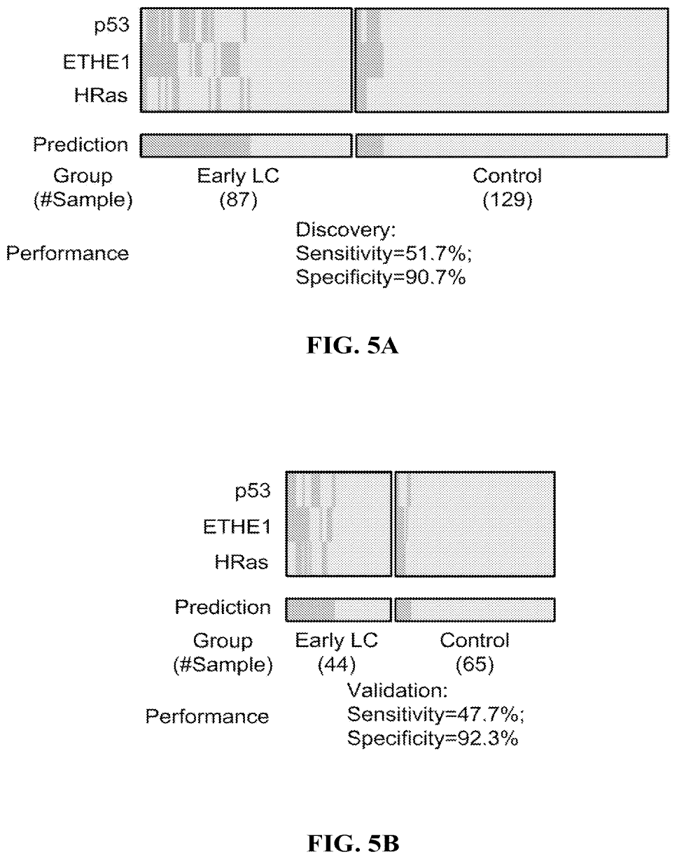

As a result, the best combination, comprised of p53, ETHE1, and HRas, achieved 50.7% sensitivity and 90.7% specificity with a k value of 1 (A serum sample would be scored positive when at least one of the three proteins showed signal intensity greater than the corresponding optimal cut off value) (FIG. 5A). When this panel was applied to the validation cohort, similar values of sensitivity and specificity were obtained (FIG. 5B), demonstrating the robustness of this panel in diagnosis of early LC. After combing the results of the discovery and validation stages, the overall sensitivity for limited SCLC and stage 1/II NSCLC is 53.3% and 49.5%, respectively.

### Example 6. Biomarker Panel Assays for Late Stage LC and Other Types of Cancer

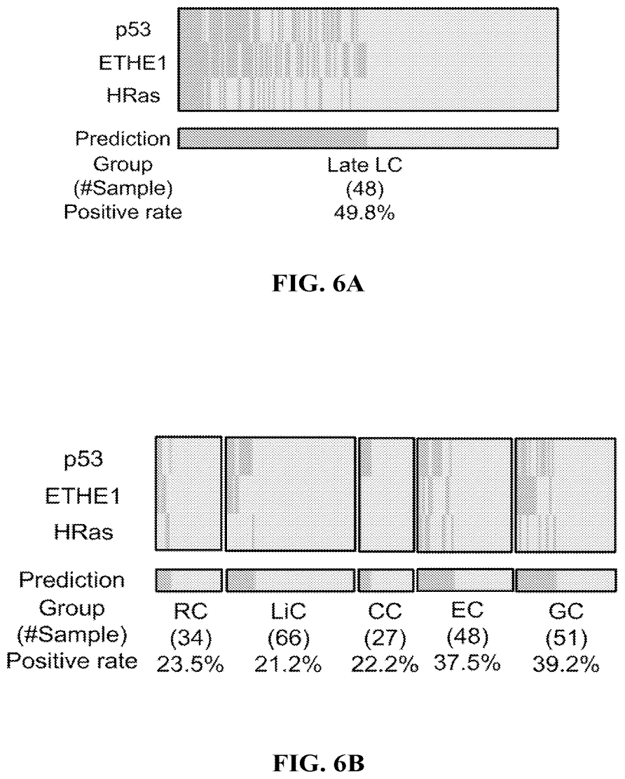

To evaluate potential value of this biomarker panel in late stage LC diagnosis, a new cohort of 221 serum samples was recruited, collected from 43 patients with extensive stage SCLC, 99 patients with stage III/IV adenocarcinoma, and 79 patients with stage III/IV squamous-cell carcinoma, and profiled them on the LC focused arrays. By applying this biomarker panel to analyze the obtained dataset, it was observed that 49.8% of the late stage LC samples could be readily detected (FIG. 6A), demonstrating that this panel of biomarkers can also be used for late stage LC diagnosis.

It is known that many known tumor antigens can be found in patients with a wide variety of cancers, diminishing their value for accurate diagnosis of specific cancer types. To assess whether the identified biomarker panel could distinguish LC from other types of cancer, a cohort of 226 serum samples, collected from 34 rectal cancer (RC), 66 liver cancer (LiC), 27 cervical cancer (CC), 48 esophagus cancer (EC), and 51 gastric cancer (GC) patients was profiled on the LC focused arrays. Interestingly, this panel of biomarkers could only detect 23.5%, 21.2%, 22.2%, 37.5%, and 39.2% of RC, LiC, CC, EC, and GC, respectively (FIG. 6B). This comparison indicated that this biomarker panel is clearly more sensitive in detecting LC.

### Example 7. ELISA Validation of Biomarker Panels

To transform these array-validated biomarkers into a more clinically friendly application, an enzyme-linked immunosorbent assay (ELISA) was developed for the three proteins in the biomarker panel. Large amounts of p53, ETHE1, and HRas proteins were purified from yeast from 300 mL of yeast culture as GST fusions and used to obtain optimized ELISA assay conditions in a 96-well format. The quality and quantity of the purified proteins were examined with Coomassie stain after SDS PAGE gel electrophoresis. 50 ng (p53), 200 ng (HRas) and 200 ng (ETHE1) in 100 μL volume of purified proteins were used to coat each well of a 96-well ELISA plate (Santa Cruz Biotechnology, USA) at 4° C. overnight, respectively. Non-specific binding was blocked by incubating with 200 μL of 3% BSA in TBST/well at 37° C. for 1 hr. Then, 100 μL of 1:500-diluted human serum samples were added to each well and incubated at 37° C. for 1 hr. After three washes with TBST (400 μL/well), 100 μL of horseradish peroxidase-labeled mouse anti-human IgG monoclonal antibody (1:50,000; Jackson ImmunoResearch, USA) was added to each well and incubated at 37° C. for 0.5 hr. After three washes with 300 μL/well of TBST, 100 μL of tetramethybenzidine substrate solution (Sigma-Aldrich, USA) was added and incubated for 10 min, followed by a termination step by adding 50 μL of 2 M H2SO4/well. The immunoreactivity signals were measured by reading the A450.

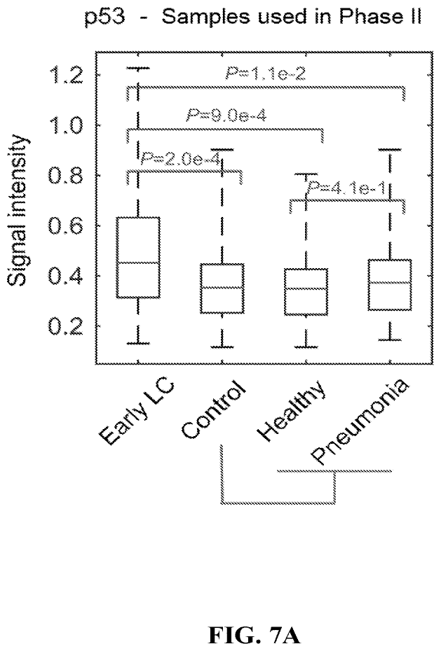

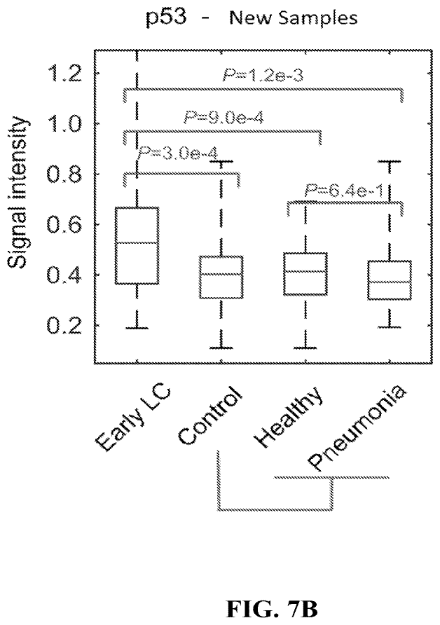

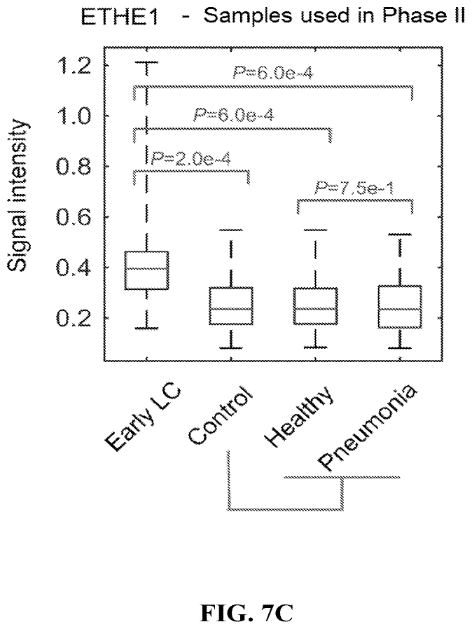

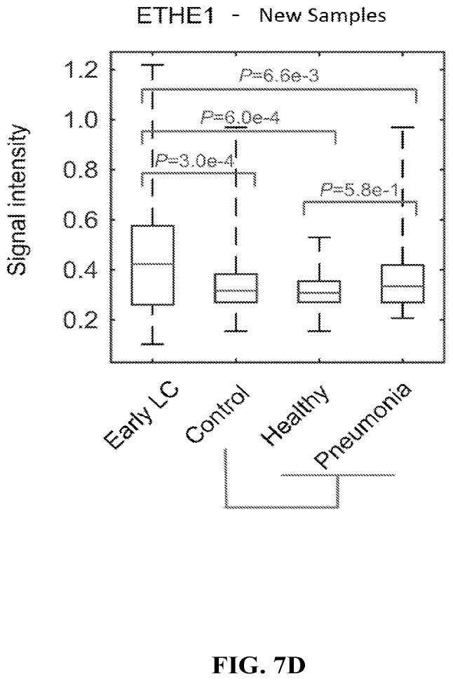

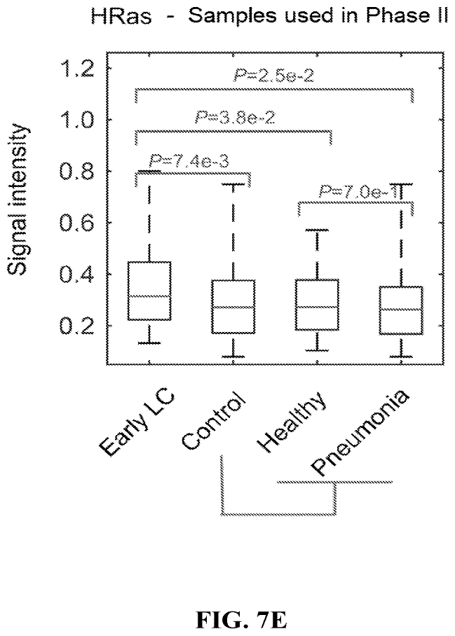

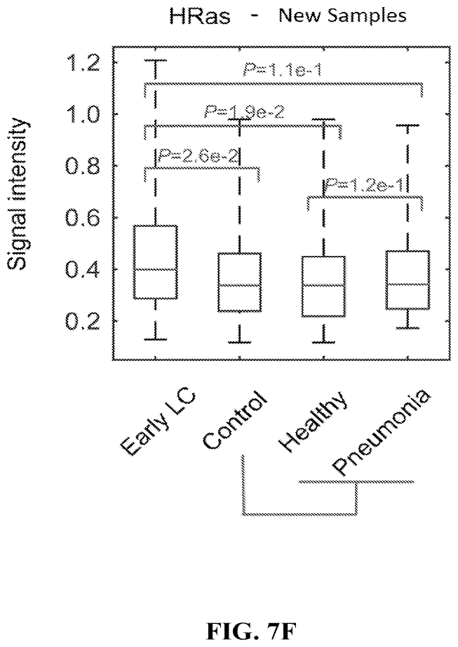

Two cohorts were assembled: one contained 226 samples randomly selected from those used in Phase II and 229 newly collected samples (FIG. 1). The ELISA data obtained with samples used in the array-based assays demonstrated that each of p53 (FIG. 7A), ETHE1 (FIG. 7C), and HRas (FIG. 7E) showed significantly higher signals in both early and late LC groups as compared with those in healthy and LBL groups. A similar result was obtained for p53 (FIG. 7B), ETHE1 (FIG. 7D), and HRas (FIG. 7F) in newly recruited cohort (FIG. 4A).

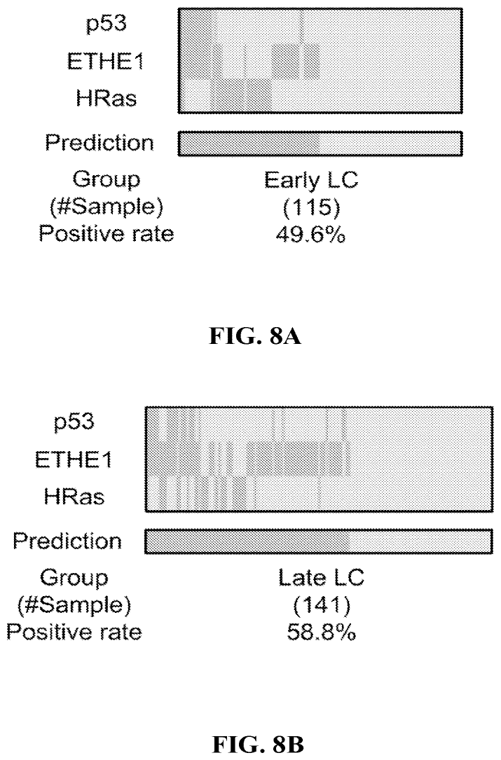

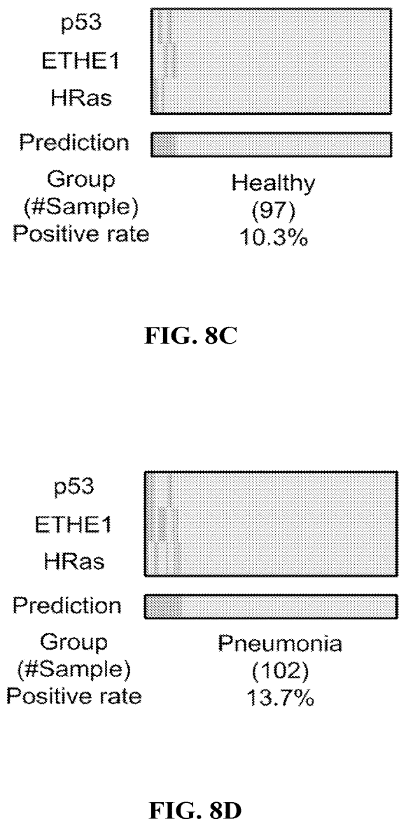

Since the above two cohorts yielded similar results, their ELISA data was combined and converted to a binary scoring system by using a cut off value of 2-S.D. above the mean of the signal intensity of the combined healthy group, following the standard ELISA protocol. Using the same criteria as described above, 49.6% of samples in the early stages of LC (FIG. 8A) and 58.8% of samples in the late stages of LC (FIG. 8B) were scored as positives, while only 10.3% of healthy samples (FIG. 8C) and 13.7% of LBL samples (FIG. 8D) were respectively scored as false positives. Sensitivity and specificity of early LC diagnosis using ELISA were determined as 49.6% and 87.9%, respectively with this biomarker panel.

To further improve the sensitivity of detecting early stage LC, serum samples scored as positives with the biomarker panel of p53/HRas/ETHE1 were removed from each group of samples and partitioned randomly to the discovery and validation cohorts with a 2:1 ratio. Using the same approach as described previously, we identified two panels (Table 7) that showed consistently good sensitivity and specificity values in the discovery and validation stages. After the p53/HRas/ETHE1 panel was combined with these two additional panels, the overall sensitivity and specificity of the newly assembled panels are 70% and 80%, respectively.

TABLE 7
Performance of two additional biomarker panels

| | Discovery | | Validation | |
|---|---|---|---|---|
| Combination | Sensitivity | Specificity | Sensitivity | Specificity |
| LIMS1, RBPJ1, RBPJ2, | 44.2% | 90.7% | 40.9% | 84.7% |
| RBPJ3, TEX264 | | | | |
| MAK16, CALCOCO2, | 41.9% | 90.7% | 18.2% | 81.4% |
| SDCBP, LGALS8, | | | | |
| DMRT2 | | | | |

### Example 8. LC Focused Array Assay for LC Bone Metastasis

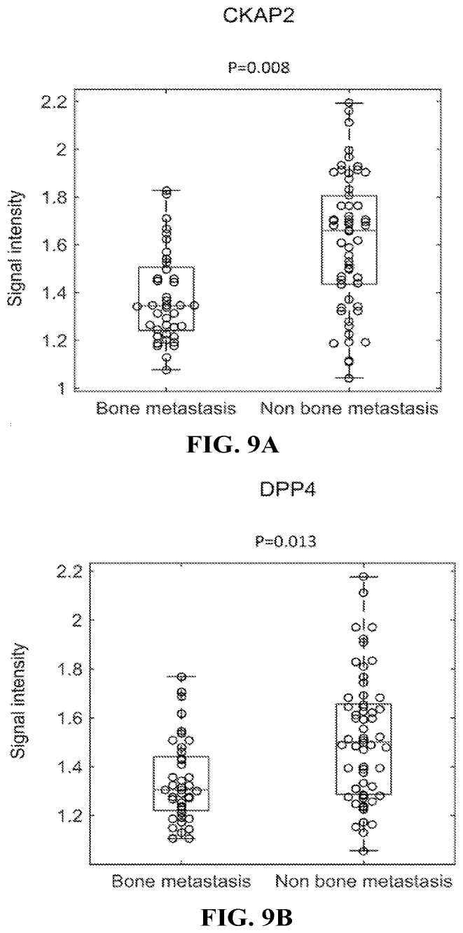

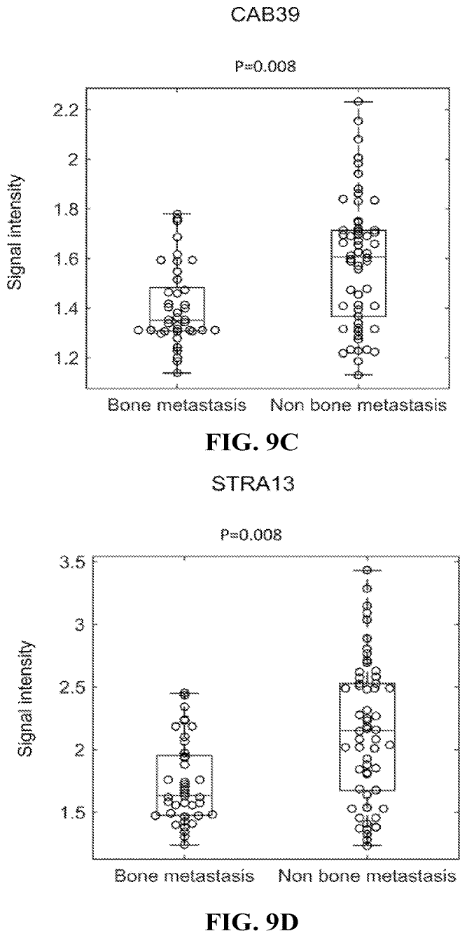

One characteristics of stage IV LC is distant metastasis, 30-40% of which are bone metastasis. Although chemotherapy and radiotherapy are commonly used for stage IV LC, they are often less effective to those patients with bone metastasis. Targeted therapy and recently developed immunotherapy are promising treatments for those with bone metastasis. Therefore, identification of biomarkers that can distinguish LC patients with bone metastasis would assist prognosis of LC treatment. Of the 99 patients in stage IV of NSCLC that were used on Phase II, 41 developed bone metastases. By comparing the human anti-IgG signal intensity obtained from the focused arrays, four proteins were found significant lower in patients with bone metastases: CKAP2 (FIG. 9A), DPP4 (FIG. 9B), CAB39 (FIG. 9C) and STRA13 (FIG. 9D).

### Example 9. LC Focused Array Assay for LC Prognosis

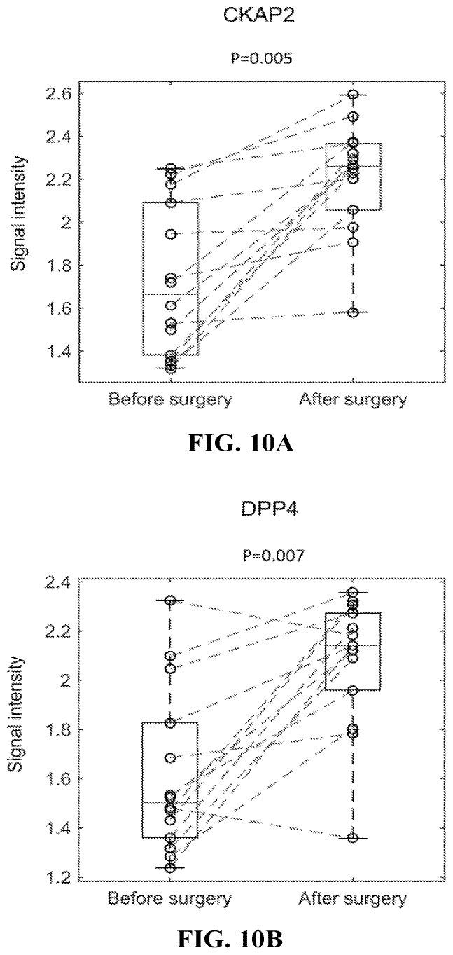

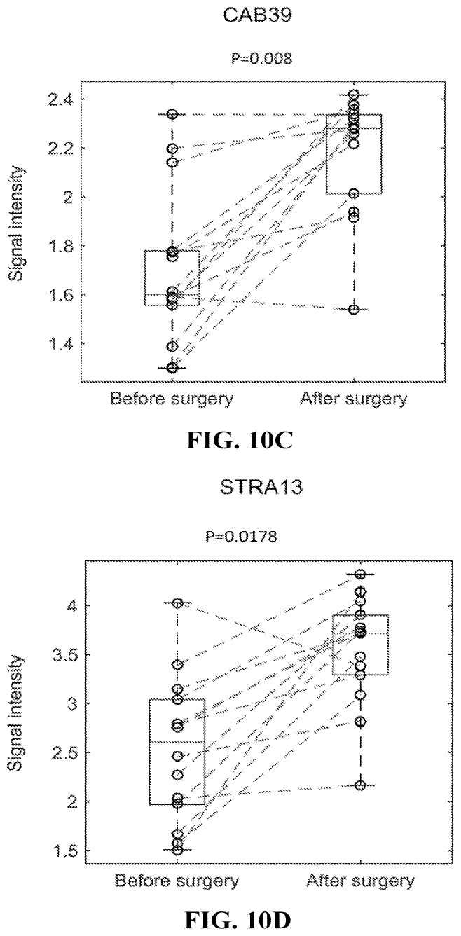

Serum samples from 13 early stage and one late stage LC patients were collected before and after surgery. By comparing the human anti-IgG signal intensity obtained from the focused arrays, each of CKAP2 (FIG. 10A), DPP4 (FIG. 10B), CAB39 (FIG. 10C) and STRA13 (FIG. 10D) showed lower signal intensity in the majority of these patients before surgery.

SEQUENCES

| | | |
|---|---|---|
| SEQ ID NO: 1 | amino acid sequence of | MAEAVLRVARRQLSQRGGSGAPILLRQMIEPVSCTFTYLL |
| | Homo sapiens ETHE1 | GDRESREAVLIDPVLETAPRDAQLIKELGLRLLYAVNTHC |
| | | HADHITGSGLLRSLLPGCQSVISRLSGAQADLHIEDGDSI |
| | | RFGRFALETRASPGHTPGCVTFVLNDHSMAFTGDALLIRG |
| | | CGRTDFQQGCAKTLYHSVHEKIFTLPGDCLIYPAHDYHGF |
| | | TVSTVEEERTLNPRLTLSCEEFVKIMGNLNLPKPQQIDFA |
| | | VPANMRCGVQTPTA |
| SEQ ID NO: 2 | amino acid sequence of | MSTPAVPQDLQLPPSQRAQSAFKEQRRQKLKEHLLRRKTL |
| | Homo sapiens CKAP2 | FAYKQENEMLSSRDQRVVTSEDQVQEGTKVLKLKTKMADK |
| | | ENMKRPAESKNNTVVGKHCIPLKPSNELTNSTVVIDTHKP |
| | | KDSNQTPHLLLlEDDPQSQHMTLSQAFHLKNNSKKKQMTT |
| | | EKQKQDANMPKKPVLGSYRGQIVQSKINSFRKPLQVKDES |
| | | SAATKKLSATIPKATKPQPVNTSSVTVKSNRSSNMTATTK |
| | | FVSTTSQNTQLVRPPIRSHHSNTRDTVKQGISRTSANVTI |
| | | RKGPHEKELLQSKTALSSVKTSSSQGIIRNKTLSRSIASE |
| | | VVARPASLSNDKLMEKSEPVDQRRHTAGKAIVDSRSAQPK |
| | | ETSEERKARLSEWKAGKGRVLKRPPNSVVTQHEPAGQNEK |
| | | PVGSFWTTMAEEDEQRLFIEKVNNTFSECLNLINEGCPKE |
| | | DILVTLNDLIKNIPDAKKLVKYWICLALIEPITSPIENII |
| | | AIYEKAILAGAQPIEEMRHTIVDILTMKSQEKANLGENME |
| | | KSCASKEEVKEVSIEDTGVDVDPEKLEMESKLHRNLLFQD |
| | | CEKEQDNKTKDPTHDVKTPNTETRTSCLIKYNVSTTPYLQ |
| | | SVKKKVQFDGTNSAFKELKFLTPVRRSRRLQEKTSKLPDM |
| | | LKDHYPCVSSLEQL1ELGRETDAFVCRPNAALCRVYYEAD |
| | | TT |
| SEQ ID NO: 3 | amino acid sequence of | MKTPWKVLLGLLGAAALVTIITVPVVLLNKGTDDATADSR |
| | Homo sapiens DPP4 | KTYTLTDYLKNTYRLKLYSLRWISDHEYLYKQENNILVFN |
| | | AEYGNSSVFLENSTFDEFGHSINDYSISPDGQFILLEYNY |
| | | VKQWRHSYTASYDIYDLNKRQLIlEERIPNNTQWVTWSPV |
| | | GHKLAYVVVNNDIYVKIEPNLPSYRITWTGKEDIIYNGIT |
| | | DWVYEEEVFSAYSALWWSPNGTFLAYAQFNDTEVPLIEYS |
| | | FYSDESLQYPKTVRVPYPKAGAVNPTVKFFVVNTDSLSSV |
| | | TNATSIQITAPASMLIGDHYLCDVTWATQERISLQWLRRI |
| | | QNYSVMDICDYDESSGRWNCLVARQHIEMSTTGWVGRFRP |
| | | SEPHFTLDGNSFYKIISNEEGYRHICYFQIDKKDCTFITK |
| | | GTWEVIGIEALTSDYLYYISNEYKGMPGGRNLYKIQLSDY |
| | | TKVTCLSCELNPERCQYYSVSFSKEAKYYQLRCSGPGLPL |
| | | YTLHSSVNDKGLRVLEDNSALDKMLQNVQMPSKKLDFIIL |
| | | NETKFWYQMILPPHFDKSKKYPLLLDVYAGPCSQKADTVF |
| | | RLNWATYLASlENIIVASFDGRGSGYQGDKIMHAINRRLG |
| | | TFEVEDQIEAARQFSKMGFVDNKRIAIWGWSYGGYVTSMV |
| | | LGSGSGVFKCGIAVAPVSRWEYYDSVYfERYMGLPTPEDN |
| | | LDHYRNSTVMSRAENFKQVEYLLIHGTADDNVHFQQSAQI |
| | | SKALVDVGVDFQAMWYTDEDHGIASSTAHQHIYTHMSHFI |
| | | KQCFSLP |
| SEQ ID NO: 4 | amino acid sequence of | MPFPFGKSHKSPADIVKNLKESMAVLEKQDISDKKAEKAT |
| | Homo sapiens CAB 39 | EEVSKNLVAMKEILYGTNEKEPQTEAVAQLAQELYNSGLL |
| | | STLVADLQLIDFEGKKDVAQIFNNILRRQIGTRTPTVEYI |
| | | CTQQNILFMLLKGYESPEIALNCGIMLRECIRHEPLAKII |
| | | LWSEQFYDFFRYVEMSTFDIASDAFATFKDLLTRHKLLSA |
| | | EFLEQHYDRFFSEYEKLLHSENYVTKRQSLKLLGELLLDR |
| | | HNFTIMTKYISKPENLKLMMNLLRDKSRNIQFEAFHVFKV |
| | | FVANPNKTQPILDILLKNQAKLIEFLSKFQNDRTEDEQFN |
| | | DEKTYLVKQIRDLKRPAQQEA |
| SEQ ID NO: 5 | amino acid sequence of | MEGAGAGSGFRKELVSRLLHLHFKDDKTKEAAVRGVRQAQ |
| | Homo sapiens STRA13 | AEDALRADVDQLEKVLPQLLLDF |
| SEQ ID NO: 6 | amino acid sequence of | MEEPQSDPSVEPPLSQETFSDLWKLLPENNVLSPLPSQAM |
| | Homo sapiens p53 | DDLMLSPDDIEQWFTEDPGPDEAPRMPEAAPRVAPAPAAP |
| | | TPAAPAPAPSWPLSSSVPSQKTYQGSYGFRLGFLHSGTAK |
| | | SVTCTYSPALNKMFCQLAKTCPVQLWVDSTPPPGTRVRAM |
| | | AIYKQSQHMTEVVRRCPHHERCSDSDGLAPPQHLIRVEGN |
| | | LRVEYLDDRNTFRHSVVVPYEPPEVGSDCTTIHYNYMCNS |
| | | SCMGGMNRRPILTIITLEDSSGNLLGRNSFEVRVCACPGR |
| | | DRRTEEENLRKKGEPHHELPPGSTKRALPNNTSSSPQPKK |
| | | KPLDGEYFTLQIRGRERFEMFRELNEALELKDAQAGKEPG |
| | | GSRAHSSHLKSKKGQSTSRHKKLMFKTEGPDSD |
| SEQ ID NO: 7 | amino acid sequence of | MQAEGRGTGGSTGDADGPGGPGIPDGPGGNAGGPGEAGAT |
| | Homo sapiens | GGRGPRGAGAARASGPGGGAPRGPHGGAASGLNGCCRCGA |
| | CTAG1A | RGPESRLLEFYLAMPFATPMEAELARRSLAQDAPPLPVPG |
| | | VLLKEFTVSGNILTIRLTAADHRQLQLSISSCLQQLSLLM |
| | | WITQCFLPVFLAQPPSGQRR |
| SEQ ID NO: 8 | amino acid sequence of | MGSRGQGLLLAYCLLLAFASGLVLSRVPHVQGEQQEWEGT |
| | Homo sapiens | EELPSPPDHAERAEEQHEKYRPSQDQGLPASRCLRCCDPG |
| | C1QTNF1 | TSMYPATAVPQINITILKGEKGDRGDRGLQGKYGKTGSAG |
| | | ARGHTGPKGQKGSMGAPGERCKSHYAAFSVGRKKPMHSNH |
| | | YYQTVIFDTEFVNLYDHFNMFTGKFYCYVPGLYFFSLNVH |
| | | TWNQKETYLHIMKNEEEVVILFAQVGDRSIMQSQSLMLEL |
| | | REQDQVWVRLYKGERENAIFSEELDTYITFSGYLVKHATE |
| | | P |
| SEQ ID NO: 9 | amino acid sequence of | MSDLLLLGLIGGLTLLLLLTLLAFAGYSGLLAGVEVSAGS |
| | Homo sapiens TEX264 | PPIRNVTVAYKFHMGLYGETGRLFTESCSISPKLRSIAVY |
| | | YDNPHMVPPDKCRCAVGSILSEGEESPSPELIDLYQKFGF |
| | | KVFSFPAPSHVVTATFPYTTILSIWLATRRVHPALDTYIK |
| | | ERKLCAYPRLEIYQEDQIHFMCPLARQGDFYVPEMKETEW |
| | | KWRGLVEAIDTQVDGTGADTMSDTSSVSLEVSPGSRETSA |
| | | ATLSPGASSRGWDDGDTRSEHSYSESGASGSSFEELDLEG |
| | | EGPLGESRLDPGIEPLGTTKWLWEPTAPEKGKE |
| SEQ ID NO: 10 | amino acid sequence of | MASLGLQLVGYILGLLGLLGTLVAMLLPSWKTSSYVGASI |
| | Homo sapiens CLDN2 | VTAVGFSKGLWMECATHSTGITQCDIYSTLLGLPADIQAA |
| | | QAMMVTSSAISSLACIISVVGMRCTVFCQESRAKDRVAVA |
| | | GGVFFILGGLLGFIPVAWNLHGILRDFYSPLVPDSMKFEI |
| | | GEALYLGIISSLFSLIAGIILCFSCSSQRNRSNYYDAYQA |
| | | QPLATRSSPRPGQPPKVKSEFNSYSLTGYV |
| SEQ ID NO: 11 | amino acid sequence of | MVKLGNNFAEKGTKQPLLEDGFDTIPLMTPLDVNQLQFPP |
| | Homo sapiens NSG1 | PDKVVVKTKIEYEPDRKKGKARPPQIAEFTVSI1EGVTER |
| | | FKVSVLVLFALAFLTCVVFLVVYKVYKYDRACPDGFVLKN |
| | | TQCIPEGLESYYAEQDSSAREKFYTVINHYNLAKQSITRS |
| | | VSPWMSVLSEEKLSEQETEAAEKSA |
| SEQ ID NO: 12 | amino acid sequence of | MTEYKLVVVGAGGVGKSALTIQLIQNHFVDEYDPTIEDSY |
| | Homo sapiens HRas | RKQVVIDGETCLLDILDTAGQEEYSAMRDQYMRTGEGFLC |
| | | VFAINNTKSFEDIHQYREQIKRVKDSDDVPMVLVGNKCDL |
| | | AARTVESRQAQDLARSYGIPYIETSAKTRQGVEDAFYTLV |
| | | REIRQHKLRKLNPPDESGPGCMSCKCVLS |
| SEQ ID NO: 13 | amino acid sequence of | MANALASATCERCKGGFAPAEKIVNSNGELYHEQCFVCAQ |
| | Homo sapiens LIMS1 | CFQQFPEGLFYEFEGRKYCEHDFQMLFAPCCHQCGEFTIG |
| | | RVIKAMNNSWHPECFRCDLCQEVLADIGFVKNAGRHLCRP |
| | | CHNREKARGLGKYICQKCHAIIDEQPLIFKNDPYHPDHFN |
| | | CANCGKELTADARELKGELYCLPCHDKMGVPICGACRRPI |
| | | EGRVVNAMGKQWHVEHFVCAKCEKPFLGHRHYERKGLAYC |
| | | ETHYNQLFGDVCFHCNRVIEGGVVSALNKAWCVNCFACST |
| | | CNTKLTLKNKFVEFDMKPVCKKCYEKFPLELKKRLKKLAE |
| | | TLGRK |
| SEQ ID NO: 14 | amino acid sequence of | MDHIEGSPAEEPPAHAPSPGKFGERPPPKRLTREAMRNYK |
| | Homo sapiens RBPJ1 | ERGDQTVLILHAKVAQKSYGNEKRFFCPPPCVYLMGSGWK |
| | | KKKEQMERDGCSEQESQPCAFIGIGNSDQEMQQLNLEGKN |
| | | YCTAKTLYISDSDKRKHFMLSVKMFYGNSDDIGVFLSKRI |
| | | KVISKPSKKKQSLKNADLCIASGTKVALFNRLRSQTVSTR |
| | | YLHVEGGNFHASSQQWGAFFIHLLDDDESEGEEFTVRDGY |
| | | IHYGQTVKLVCSVTGMALPRLIIRKVDKQTALLDADDPVS |
| | | QLHKCAFYLKDIERMYLCLSQERIIQFQATPCPKEPNKEM |
| | | INDGASWTIISTDKAEYTFYEGMGPVLAPVTPVPVVESLQ |
| | | LNGGGDVAMLELTGQNFTPNLRVVVFGDVEAETMYRCGES |
| | | MLCVVPDISAFREGWRWVRQPVQVPVTLVRNDGIIYSTSL |
| | | TFTYTPEPGPRPHCSAAGAILRANSSQVPPNESNTNSEGS |
| | | YTNASTNSTSVTSSTATVVS |
| SEQ ID NO: 15 | amino acid sequence of | MAPVVTGKFGERPPPKRLTREAMRNYLKERGDQTVLILHA |
| | Homo sapiens RBPJ2 | KVAQKSYGNEKRFFCPPPCVYLMGSGWKKKKEQMERDGCS |
| | | EQESQPCAFIGIGNSDQEMQQLNLEGKNYCTAKTLYISDS |
| | | DKRKHFMLSVKMFYGNSDDIGVFLSKRIKVISKPSKKKQS |
| | | LKNADLCIASGTKVALFNRLRSQTVSTRYLHVEGGNFHAS |
| | | SQQWGAFFIHLLDDDESEGEEFTVRDGYIHYGQTVKLVCS |
| | | VTGMALPRLIIRKVDKQTALLDADDPVSQLHKCAFYLKDI |
| | | ERMYLCLSQERIIQFQATPCPKEPNKEMINDGASWTIIST |
| | | DKAEYTFYEGMGPVLAPVTPVPVVESLQLNGGGDVAMLEL |
| | | TGQNFTPNLRVWFGDVEAETMYRCGESMLCVVPDISAFRE |
| | | GWRWVRQPVQVPVTLVRNDGIIYSTSLTFTYTPEPGPRPH |
| | | CSAAGAILRANSSQVPPNESNTNSEGSYTNASTNSTSVTS |
| | | STATVVS |
| SEQ ID NO: 16 | amino acid sequence of | MGGCRKFGERPPPKRLTREAMRNYLKERGDQTVLILHAKV |
| | Homo sapiens RBPJ3 | AQKSYGNEKRFFCPPPCVYLMGSGWKKKKEQMERDGCSEQ |
| | | ESQPCAFIGIGNSDQEMQQLNLEGKNYCTAKTLYISDSDK |
| | | RKHFMLSVKMFYGNSDDIGVFLSKRIKVISKPSKKKQSLK |
| | | NADLCIASGTKVALFNRLRSQTVSTRYLHVEGGNFHASSQ |
| | | QWGAFFIHLLDDDESEGEEFTVRDGYIHYGQTVKLVCSVT |
| | | GMALPRLIIRKVDKQTALLDADDPVSQLHKCAFYLKDTER |
| | | MYLCLSQERIIQFQATPCPKEPNKEMINDGASWTIISTDK |
| | | AEYTFYEGMGPVLAPVTPVPVVESLQLNGGGDVAMLELTG |
| | | QNFTPNLRVWFGDVEAETMYRCGESMLCVVPDISAFREGW |
| | | RWVRQPVQVPVTLVRNDGIIYSTSLTFTYTPEPGPRPHCS |
| | | AAGAILRANSSQVPPNESNTNSEGSYTNASTNSTSVTSST |
| | | ATVVS |
| SEQ ID NO: 17 | amino acid sequence of | MQSDDVIWDTLGNKQFCSFKIRTKTQSFCRNEYSLTGLCN |
| | Homo sapiens MAK16 | RSSCPLANSQYATIKEEKGQCYLYMKVIERAAFPRRLWER |
| | | VRLSKNYEKALEQIDENLIYWPRFIRHKCKQRFTKITQYL |
| | | IRIRKLTLKRQRKLVPLSKKVERREKRREEKALIAAQLDN |
| | | AIEKELLERLKQDTYGDIYNFPIHAFDKALEQQEAESDSS |
| | | DTEEKDDDDDDEEDVGKREFVEDGEVDESDISDFEDMDKL |
| | | DASSDEDQDGKSSSEEEEEKALSAKHKGKMPLRGPLQRKR |
| | | AYVEIEYEQETEPVAKAKTT |
| SEQ ID NO: 18 | amino acid sequence of | MEETIKDPPTSAVLLDHCHFSQVIFNSVEKFYIPGGDVTC |
| | Homo sapiens | HYTFTQHFIPRRKDWIGIFRVGWKTTREYYTFMWVTLPID |
| | CALCOCO2 | LNNKSAKQQEVQFKAYYLPKDDEYYQFCYVDEDGVVRGAS |
| | | IPFQFRPENEEDILVVTTQGEVEEIEQHNKELCKENQELK |
| | | DSCISLQKQNSDMQAELQKKQEELETLQSINKKLELKVKE |
| | | QKDYWEIELLQLKEQNQKMSSENEKMGIRVDQLQAQLSTQ |
| | | EKEMEKLVQGDQDKTEQLEQLKKENDHLFLSLTEQRKDQK |
| | | KLEQTVEQMKQNETTAMKKQQELMDENFDLSKRLSENEII |
| | | CNALQRQKERLEGENDLLKRENSRLLSYMGLDFNSLPYQV |
| | | PTSDEGGARQNPGLAYGNPYSGIQESSSASPLSIKKCPIC |
| | | KADDICDHTLEQQQMQPLCFNCPICDKIFPATEKQIFEDH |
| | | VFCHSL |
| SEQ ID NO: 19 | amino acid sequence of | MSLYPSLEDLKVDKVIQAQTAFSANPANPAILSEASAPIP |
| | Homo sapiens SDCBP | HDGNLYPRLYPELSQYMGLSLNEEEIRANVAVVSGAPLQG |
| | | QLVARPSSINYMVAPVTGNDVGIRRAEIKQGIREVILCKD |
| | | QDGKIGLRLKSIDNGIFVQLVQANSPASLVGLRFGDQVLQ |
| | | INGENCAGWSSDKAHKVLKQAFGEKITMTIRDRPFERTIT |
| | | MHKDSTGHVGFIFKNGKITSIVKDSSAARNGLLTEHNICE |
| | | INGQNVIGLKDSQIADILSTSGTVVTITIMPAFIFEHIIK |
| | | RMAPSIMKSLMDHTIPEV |
| SEQ ID NO: 20 | amino acid sequence of | MMLSLNNLQNIIYNPVIPFVGTIPDQLDPGTLIVIRGHVP |
| | Homo sapiens LGALS8 | SDADRFQVDLQNGSSMKPRADVAFHFNPRFKRAGCIVCNT |
| | | LINEKWGREEITYDTPFKREKSFEIVIMVLKDKFQVAVNG |
| | | KHTLLYGHRIGPEKIDTLGIYGKVNIHSIGFSFSSDLQST |
| | | QASSLELTEISRENVPKSGTPQLPSNRGGDISKIAPRTVY |
| | | TKSKDSTVNHTLTCTKIPPMNYVSKRLPFAARLNTPMGPG |
| | | RTVVVKGEVNANAKSFNVDLLAGKSKDIALHLNPRLNIKA |
| | | FVRNSFLQESWGEEERNITSFPFSPGMYFEMIIYCDVREF |
| | | KVAVNGVHSLEYKHRFKELSSIDTLEINGDIHLLEVRSW |
| SEQ ID NO: 21 | amino acid sequence of | MADPQAGSAAGDWEIDVESLELEEDVCGAPRSTPPGPSPP |
| | Homo sapiens DMRT2 | PADGDCEDDEDDDGVDEDAEEEGDGEEAGASPGMPGQPEQ |
| | | RGGPQPRPPLAPQASPAGTGPRERCTPAGGGAEPRKLSRT |
| | | PKCARCRNHGVVSCLKGHKRFCRWRDCQCANCLLVVERQR |
| | | VMAAQVALRRQQATEDKKGLSGKQNNFERKAVYQRQVRAP |
| | | SLLAKSILEVLLGLFYSYYVYIMNHL |
| SEQ ID NO: 22 | antigenic sequence for | AVLRVARR |
| | ETHE1 | |
| SEQ ID NO: 23 | antigenic sequence for | GAPILLR |
| | ETHE1 | |
| SEQ ID NO: 24 | antigenic sequence for | MIEPVSCTFTYLL |
| | ETHE1 | |
| SEQ ID NO: 25 | antigenic sequence for | REAVLIDPVLE |
| | ETHE1 | |
| SEQ ID NO: 26 | antigenic sequence for | HTPGCVTFVLN |
| | ETHE1 | |
| SEQ ID NO: 27 | antigenic sequence for | TGDALLIR |
| | ETHE1 | |
| SEQ ID NO: 28 | antigenic sequence for | NPRLTLSCEEFVKI |
| | ETHE1 | |
| SEQ ID NO: 29 | antigenic sequence for | NLPKPQQIDFAVPA |
| | ETHE1 | |
| SEQ ID NO: 30 | antigenic sequence for | PAVPQDLQLPPSQRAQS |
| | CKAP2 | |
| SEQ ID NO: 31 | antigenic sequence for | LKEHLLRR |
| | CKAP2 | |
| SEQ ID NO: 32 | antigenic sequence for | RDQRVVTSE |
| | CKAP2 | |
| SEQ ID NO: 33 | antigenic sequence for | EGTKVLKL |
| | CKAP2 | |
| SEQ ID NO: 34 | antigenic sequence for | NNTVVGKHCIPL |
| | CKAP2 | |
| SEQ ID NO: 35 | antigenic sequence for | TNSTVVID |
| | CKAP2 | |
| SEQ ID NO: 36 | antigenic sequence for | QTPHLLLT |
| | CKAP2 | |
| SEQ ID NO: 37 | antigenic sequence for | QHMTLSQAF |
| | CKAP2 | |
| SEQ ID NO: 38 | antigenic sequence for | KKPVLGS |
| | CKAP2 | |
| SEQ ID NO: 39 | antigenic sequence for | RGQIVQS |
| | CKAP2 | |
| SEQ ID NO: 40 | antigenic sequence for | RKPLQVKD |
| | CKAP2 | |
| SEQ ID NO: 41 | antigenic sequence for | ATKKLSATI |
| | CKAP2 | |
| SEQ ID NO: 42 | antigenic sequence for | KPQPVNTSSVTV |
| | CKAP2 | |
| SEQ ID NO: 43 | antigenic sequence for | TTKFVST |
| | CKAP2 | |
| SEQ ID NO: 44 | antigenic sequence for | NTQLVRPPIRS |
| | CKAP2 | |
| SEQ ID NO: 45 | antigenic sequence for | EKELLQSKTALSSVKT |
| | CKAP2 | |
| SEQ ID NO: 46 | antigenic sequence for | IASEVVARPAS |
| | CKAP2 | |
| SEQ ID NO: 47 | antigenic sequence for | AGKAIVDS |
| | CKAP2 | |
| SEQ ID NO: 48 | antigenic sequence for | PPNSVVTQ |
| | CKAP2 | |
| SEQ ID NO: 49 | antigenic sequence for | PKEDILVTLN |
| | CKAP2 | |
| SEQ ID NO: 50 | antigenic sequence for | DAKKLVKYWICLALIEPIT |
| | CKAP2 | |
| SEQ ID NO: 51 | antigenic sequence for | IENIIAIYEKAILAGA |
| | CKAP2 | |
| SEQ ID NO: 52 | antigenic sequence for | RHTIVDI |
| | CKAP2 | |
| SEQ ID NO: 53 | antigenic sequence for | EVKEVSIE |
| | CKAP2 | |
| SEQ ID NO: 54 | antigenic sequence for | TGVDVDP |
| | CKAP2 | |
| SEQ ID NO: 55 | antigenic sequence for | LHRNLLFQD |
| | CKAP2 | |
| SEQ ID NO: 56 | antigenic sequence for | FKELKFLTP |
| | CKAP2 | |
| SEQ ID NO: 57 | antigenic sequence for | LKDHYPCVSSLEQ |
| | CKAP2 | |
| SEQ ID NO: 58 | antigenic sequence for | TDAFVCRPNAALCRVYY |
| | CKAP2 | |
| SEQ ID NO: 59 | antigenic sequence for | YRLKLYSLRW |
| | DPP4 | |
| SEQ ID NO: 60 | antigenic sequence for | SDHEYLY |
| | DPP4 | |
| SEQ ID NO: 61 | antigenic sequence for | NNILVFN |
| | DPP4 | |
| SEQ ID NO: 62 | antigenic sequence for | GNSSVFL |
| | DPP4 | |
| SEQ ID NO: 63 | antigenic sequence for | PDGQFILLEYNYVKQ |
| | DPP4 | |
| SEQ ID NO: 58 | antigenic sequence for | HSYTASYDIYD |
| | DPP4 | |
| SEQ ID NO: 64 | antigenic sequence for | WVTWSPVGHKLAY |
| | DPP4 | |
| SEQ ID NO: 65 | antigenic sequence for | WNNDIYVKIEPNLPSY |
| | DPP4 | |
| SEQ ID NO: 66 | antigenic sequence for | YEEEVFSAYSAL |
| | DPP4 | |
| SEQ ID NO: 67 | antigenic sequence for | GTFLAYA |
| | DPP4 | |
| SEQ ID NO: 68 | antigenic sequence for | SLSSVTN |
| | DPP4 | |
| SEQ ID NO: 69 | antigenic sequence for | ERISLQWL |
| | DPP4 | |
| SEQ ID NO: 70 | antigenic sequence for | QNYSVMDICDY |
| | DPP4 | |
| SEQ ID NO: 71 | antigenic sequence for | RWNCLVAR |
| | DPP4 | |
| SEQ ID NO: 72 | antigenic sequence for | YRHICYFQ |
| | DPP4 | |
| SEQ ID NO: 73 | antigenic sequence for | DKKDCTF |
| | DPP4 | |
| SEQ ID NO: 74 | antigenic sequence for | EVIGIEALTSDYLYYIS |
| | DPP4 | |
| SEQ ID NO: 75 | antigenic sequence for | GLRVLED |
| | DPP4 | |
| SEQ ID NO: 76 | antigenic sequence for | MLQNVQMP |
| | DPP4 | |
| SEQ ID NO: 77 | antigenic sequence | KKLDFIIL |
| | foDPP4 | |
| SEQ ID NO: 78 | antigenic sequence for | WYQMILPPH |
| | DPP4 | |
| SEQ ID NO: 79 | antigenic sequence for | WATYLAS |
| | DPP4 | |
| SEQ ID NO: 80 | antigenic sequence for | ENIIVAS |
| | DPP4 | |
| SEQ ID NO: 81 | antigenic sequence for | YYDSVYT |
| | DPP4 | |
| SEQ ID NO: 82 | antigenic sequence for | KQVEYLLIH |
| | DPP4 | |
| SEQ ID NO: 83 | antigenic sequence for | HKSPADIVKN |
| | CAB39 | |
| SEQ ID NO: 84 | antigenic sequence for | ESMAVLEK |
| | CAB39 | |
| SEQ ID NO: 85 | antigenic sequence for | VSKNLVAMKEI |
| | CAB39 | |
| SEQ ID NO: 86 | antigenic sequence for | QTEAVAQLAQE |
| | CAB39 | |
| SEQ ID NO: 87 | antigenic sequence for | YNSGLLSTLVADLQLID |
| | CAB39 | |
| SEQ ID NO: 88 | antigenic sequence for | KDVAQIFN |
| | CAB39 | |
| SEQ ID NO: 89 | antigenic sequence for | PTVEYICTQQNILFMLLKG |
| | CAB39 | |
| SEQ ID NO: 90 | antigenic sequence for | QFYDFFRYVE |
| | CAB39 | |
| SEQ ID NO: 91 | antigenic sequence for | EYEKLLHSEN |
| | CAB39 | |
| SEQ ID NO: 92 | antigenic sequence for | QSLKLLGELLLD |
| | CAB39 | |
| SEQ ID NO: 93 | antigenic sequence for | QFEAFHVFKVFVA |
| | CAB39 | |
| SEQ ID NO: 94 | antigenic sequence for | TQPILDILLKNQAKLIEFLS |
| | CAB39 | |
| SEQ ID NO: 95 | antigenic sequence for | KTYLVKQI |
| | CAB39 | |
| SEQ ID NO: 96 | antigenic sequence for | RKELVSRLLHLHF |
| | STRA13 | |
| SEQ ID NO: 97 | antigenic sequence for | KEAAVRGVRQ |
| | STRA13 | |
| SEQ ID NO: 98 | antigenic sequence for | LRADVDQLEKVLPQL |
| | STRA13 | |
| SEQ ID NO: 99 | antigenic sequence for | SDPSVEPPLSQ |
| | P53 | |
| SEQ ID NO: 101 | antigenic sequence for | PENNVLSPLPS |
| | P53 | |
| SEQ ID NO: 102 | antigenic sequence for | YGFRLGFLHS |
| | P53 | |
| SEQ ID NO: 103 | antigenic sequence for | TAKSVTCTYSP |
| | P53 | |
| SEQ ID NO: 104 | antigenic sequence for | LNKMFCQLAKTCPVQLWVDS |
| | p53 | |
| SEQ ID NO: 105 | antigenic sequence for | RAMAIYKQ |
| | P53 | |
| SEQ ID NO: 106 | antigenic sequence for | HMTEVVRRCPHHER |
| | P53 | |
| SEQ ID NO: 107 | antigenic sequence for | GLAPPQHLIRV |
| | P53 | |
| SEQ ID NO: 108 | antigenic sequence for | PILTIITL |
| | P53 | |
| SEQ ID NO: 109 | antigenic sequence for | FEVRVCACP |
| | P53 | |
| SEQ ID NO: 110 | antigenic sequence for | PHHELPP |
| | P53 | |
| SEQ ID NO: 111 | antigenic sequence for | DGEYFTL |
| | P53 | |
| SEQ ID NO: 112 | antigenic sequence for | AHSSHLK |
| | P53 | |
| SEQ ID NO: 113 | antigenic sequence for | YKLVVVGA |
| | HRas | |
| SEQ ID NO: 114 | antigenic sequence for | GVGKSALTIQLIQNHFVDE |
| | HRas | |
| SEQ ID NO: 115 | antigenic sequence for | YRKQVVIDGETCLLDI |
| | HRas | |
| SEQ ID NO: 116 | antigenic sequence for | EGFLCVFA |
| | HRas | |
| SEQ ID NO: 117 | antigenic sequence for | FEDIHQY |
| | HRas | |
| SEQ ID NO: 118 | antigenic sequence for | DVPMVLVGNKCDLAAR |
| | HRas | |
| SEQ ID NO: 119 | antigenic sequence for | QAQDLARSYGIPYIE |
| | HRas | |
| SEQ ID NO: 120 | antigenic sequence for | VEDAFYTLVRE |
| | HRas | |

1-178. (canceled)

179. A method of characterizing a biological sample, said method comprising:
contacting a biological sample to a plurality of isolated and purified tumor-associated proteins or fragments thereof, said plurality of isolated and purified tumor-associated proteins or fragments thereof comprising a Testis Expressed 264 (TEX264) protein or fragment thereof, an ethylmalonic encephalopathy protein 1 (ETHE1) protein or fragment thereof, and a GTPase HRas (HRAS) protein or fragment thereof; and
detecting a binding of an antibody to one or more of the TEX264 protein or fragment thereof, the ETHE1 protein or fragment thereof, and the HRAS protein or fragment thereof.

180. The method of claim 179, wherein the antibody is an anti-TEX264 IgG, an anti-ETHE1 IgG, or an anti-HRAS IgG.

181. The method of claim 179, comprising detecting binding of an anti-TEX264 antibody to the TEX264 protein or fragment thereof, detecting binding of an anti-ETHE1 antibody to the ETHE1 protein or fragment thereof, and detecting binding of an anti-HRAS antibody to the HRAS protein or fragment thereof.

182. The method of claim 179, comprising detecting binding of an anti-TEX264 IgG to the TEX264 protein or fragment thereof, detecting binding of an anti-ETHE1 IgG to the ETHE1 protein or fragment thereof, and detecting binding of an anti-HRAS IgG to the HRAS protein or fragment thereof.

183. The method of claim 179, wherein said plurality of isolated and purified tumor-associated proteins or fragments thereof is provided on a solid support.

184. The method of claim 179, further comprising identifying the biological sample as being obtained from a subject having a cancer or from a subject in need of a treatment for a cancer.

185. The method of claim 184, wherein the identifying is performed with a sensitivity of at least 40%.

186. The method of claim 184, wherein the identifying is performed with a specificity of at least 90%.

187. The method of claim 179, wherein the biological sample is a body fluid.

188. The method of claim 179, wherein the biological sample is a blood sample or a fraction thereof.

189. The method of claim 179, wherein the biological sample is a human sample.

190. The method of claim 179, wherein the plurality of isolated and purified tumor-associated proteins or fragments thereof comprises less than 100 sequentially unique purified tumor-associated proteins or fragments thereof.

191. The method of claim 179, wherein the plurality of isolated and purified tumor-associated proteins or fragments thereof comprises less than 10 sequentially unique purified tumor-associated proteins or fragments thereof.

192. The method of claim 179, wherein the antibody is an autoantibody.

193. The method of claim 179, wherein the antibody binds to at least a portion of an antigenic sequence provided by SEQ ID NO: 1, 9, and/or 12.

194. The method of claim 179, wherein the antibody binds to at least a portion of an antigenic sequence having at least 90% sequence identity to SEQ ID NO: 1, 9, and/or 12 as determined by a sequence alignment performed using BLAST.

195. The method of claim 179, wherein the detecting comprises detecting a signal.

196. The method of claim 195, wherein the signal is detected by or after associating the antibody with a probe.

197. The method of claim 196, wherein the probe is directly or indirectly associated with the antibody.

198. The method of claim 196, wherein the probe is an anti-immunoglobulin antibody.

199. The method of claim 196, wherein the probe comprises or is associated with a chromophore.

200. The method of claim 199, wherein the chromophore comprises a fluorescent marker.

201. The method of claim 195, wherein the signal has a Z score greater than a cutoff value of 1.

202. The method of claim 195, wherein the signal is detected if the signal is at least about 2 standard deviations greater than a reference signal.

203. The method of claim 202, wherein the reference signal is detected after contacting a second sample from a second subject with the plurality of isolated and purified tumor-associated proteins or fragments thereof.

204. The method of claim 202, wherein the second subject is a non-diseased human subject.

205. The method of claim 179, wherein said plurality of isolated and purified tumor-associated proteins or fragments thereof is provided on a bead.

206. The method of claim 179, wherein one or more isolated and purified tumor-associated proteins or fragments thereof comprises a tag.

207. The method of claim 183, wherein the solid support is coded for identification purposes.

208. The method of claim 205, wherein the bead is coded for identification purposes.

209. The method of claim 205, wherein the bead further comprises a chromophore.


SEQUENCE LISTING (121 sequences)

SEQ ID NO: 1 - protein, 254 aa, Homo sapiens
MAEAVLRVARRQLSQRGGSGAPILLRQMFEPVSCTFTYLLGDRESREAVLIDPVLETAPRDAQLIKELGLRLLYAVNTHCHADHITGSGLLRSLLPGCQSVISRLSGAQADLHIEDGDSIRFGRFALETRASPGHTPGCVTFVLNDHSMAFTGDALLIRGCGRTDFQQGCAKTLYHSVHEKIFTLPGDCLIYPAHDYHGFTVSTVEEERTLNPRLTLSCEEFVKIMGNLNLPKPQQIDFAVPANMRCGVQTPTA

SEQ ID NO: 2 - protein, 682 aa, Homo sapiens [sequence omitted]

SEQ ID NO: 3 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 4 - protein, 341 aa, Homo sapiens
MPFPFGKSHKSPADIVKNLKESMAVLEKQDISDKKAEKATEEVSKNLVAMKEILYGTNEKEPQTEAVAQLAQELYNSGLLSTLVADLQLIDFEGKKDVAQIFNNILRRQIGTRTPTVEYICTQQNILFMLLKGYESPEIALNCGIMLRECIRHEPLAKIILWSEQFYDFFRYVEMSTFDIASDAFATFKDLLTRHKLLSAEFLEQHYDRFFSEYEKLLHSENYVTKRQSLKLLGELLLDRHNFTIMTKYISKPENLKLMMNLLRDKSRNIQFEAFHVFKVFVANPNKTQPILDILLKNQAKLIEFLSKFQNDRTEDEQFNDEKTYLVKQIRDLKRPAQQEA

SEQ ID NO: 5 - protein, 63 aa, Homo sapiens
MEGAGAGSGFRKELVSRLLHLHFKDDKTKEAAVRGVRQAQAEDALRADVDQLEKVLPQLLLDF

SEQ ID NO: 6 - protein, 393 aa, Homo sapiens [sequence omitted]

SEQ ID NO: 7 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 8 - protein, 281 aa, Homo sapiens
MGSRGQGLLLAYCLLLAFASGLVLSRVPHVQGEQQEWEGTEELPSPPDHAERAEEQHEKYRPSQDQGLPASRCLRCCDPGTSMYPATAVPQINITILKGEKGDRGDRGLQGKYGKTGSAGARGHTGPKGQKGSMGAPGERCKSHYAAFSVGRKKPMHSNHYYQTVIFDTEFVNLYDHFNMFTGKFYCYVPGLYFFSLNVHTWNQKETYLHIMKNEEEVVILFAQVGDRSIMQSQSLMLELREQDQVWVRLYKGERENAIFSEELDTYITFSGYLVKHATEP

SEQ ID NO: 9 - protein, 313 aa, Homo sapiens
MSDLLLLGLIGGLTLLLLLTLLAFAGYSGLLAGVEVSAGSPPIRNVTVAYKFHMGLYGETGRLFTESCSISPKLRSIAVYYDNPHMVPPDKCRCAVGSILSEGEESPSPELIDLYQKFGFKVFSFPAPSHVVTATFPYTTILSIWLATRRVHPALDTYIKERKLCAYPRLEIYQEDQIHFMCPLARQGDFYVPEMKETEWKWRGLVEAIDTQVDGTGADTMSDTSSVSLEVSPGSRETSAATLSPGASSRGWDDGDTRSEHSYSESGASGSSFEELDLEGEGPLGESRLDPGTEPLGTTKWLWEPTAPEKGKE

SEQ ID NO: 10 - protein, 230 aa, Homo sapiens
MASLGLQLVGYILGLLGLLGTLVAMLLPSWKTSSYVGASIVTAVGFSKGLWMECATHSTGITQCDIYSTLLGLPADIQAAQAMMVTSSAISSLACIISVVGMRCTVFCQESRAKDRVAVAGGVFFILGGLLGFIPVAWNLHGILRDFYSPLVPDSMKFEIGEALYLGIISSLFSLIAGIILCFSCSSQRNRSNYYDAYQAQPLATRSSPRPGQPPKVKSEFNSYSLTGYV

SEQ ID NO: 11 - protein, 185 aa, Homo sapiens
MVKLGNNFAEKGTKQPLLEDGFDTIPLMTPLDVNQLQFPPPDKVVVKTKTEYEPDRKKGKARPPQIAEFTVSITEGVTERFKVSVLVLFALAFLTCVVFLVVYKVYKYDRACPDGFVLKNTQCIPEGLESYYAEQDSSAREKFYTVINHYNLAKQSITRSVSPWMSVLSEEKLSEQETEAAEKSA

SEQ ID NO: 12 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 13 - protein, 325 aa, Homo sapiens
MANALASATCERCKGGFAPAEKIVNSNGELYHEQCFVCAQCFQQFPEGLFYEFEGRKYCEHDFQMLFAPCCHQCGEFTIGRVIKAMNNSWHPECFRCDLCQEVLADIGFVKNAGRHLCRPCHNREKARGLGKYICQKCHAIIDEQPLIFKNDPYHPDHFNCANCGKELTADARELKGELYCLPCHDKMGVPICGACRRPIEGRVVNAMGKQWHVEHFVCAKCEKPFLGHRHYERKGLAYCETHYNQLFGDVCFHCNRVIEGGVVSALNKAWCVNCFACSTCNTKLTLKNKFVEFDMKPVCKKCYEKFPLELKKRLKKLAETLGRK

SEQ ID NO: 14 - protein, 499 aa, Homo sapiens
MDHTEGSPAEEPPAHAPSPGKFGERPPPKRLTREAMRNYKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFFIHLLDDDESEGEEFTVRDGYIHYGQTVKLVCSVTGMALPRLIIRKVDKQTALLDADDPVSQLHKCAFYLKDTERMYLCLSQERIIQFQATPCPKEPNKEMINDGASWTIISTDKAEYTFYEGMGPVLAPVTPVPVVESLQLNGGGDVAMLELTGQNFTPNLRVWFGDVEAETMYRCGESMLCVVPDISAFREGWRWVRQPVQVPVTLVRNDGIIYSTSLTFTYTPEPGPRPHCSAAGAILRANSSQVPPNESNTNSEGSYTNASTNSTSVTSSTATVVS

SEQ ID NO: 15 - protein, 487 aa, Homo sapiens
MAPVVTGKFGERPPPKRLTREAMRNYLKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFFIHLLDDDESEGEEFTVRDGYIHYGQTVKLVCSVTGMALPRLIIRKVDKQTALLDADDPVSQLHKCAFYLKDTERMYLCLSQERIIQFQATPCPKEPNKEMINDGASWTIISTDKAEYTFYEGMGPVLAPVTPVPVVESLQLNGGGDVAMLELTGQNFTPNLRVWFGDVEAETMYRCGESMLCVVPDISAFREGWRWVRQPVQVPVTLVRNDGIIYSTSLTFTYTPEPGPRPHCSAAGAILRANSSQVPPNESNTNSEGSYTNASTNSTSVTSSTATVVS

SEQ ID NO: 16 - protein, 485 aa, Homo sapiens
MGGCRKFGERPPPKRLTREAMRNYLKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFFIHLLDDDESEGEEFTVRDGYIHYGQTVKLVCSVTGMALPRLIIRKVDKQTALLDADDPVSQLHKCAFYLKDTERMYLCLSQERIIQFQATPCPKEPNKEMINDGASWTIISTDKAEYTFYEGMGPVLAPVTPVPVVESLQLNGGGDVAMLELTGQNFTPNLRVWFGDVEAETMYRCGESMLCVVPDISAFREGWRWVRQPVQVPVTLVRNDGIIYSTSLTFTYTPEPGPRPHCSAAGAILRANSSQVPPNESNTNSEGSYTNASTNSTSVTSSTATVVS

SEQ ID NO: 17 - protein, 300 aa, Homo sapiens
MQSDDVIWDTLGNKQFCSFKIRTKTQSFCRNEYSLTGLCNRSSCPLANSQYATIKEEKGQCYLYMKVIERAAFPRRLWERVRLSKNYEKALEQIDENLIYWPRFIRHKCKQRFTKITQYLIRIRKLTLKRQRKLVPLSKKVERREKRREEKALIAAQLDNAIEKELLERLKQDTYGDIYNFPIHAFDKALEQQEAESDSSDTEEKDDDDDDEEDVGKREFVEDGEVDESDISDFEDMDKLDASSDEDQDGKSSSEEEEEKALSAKHKGKMPLRGPLQRKRAYVEIEYEQETEPVAKAKTT

SEQ ID NO: 18 - protein, 446 aa, Homo sapiens [sequence omitted]

SEQ ID NO: 19 - protein, 298 aa, Homo sapiens
MSLYPSLEDLKVDKVIQAQTAFSANPANPAILSEASAPIPHDGNLYPRLYPELSQYMGLSLNEEEIRANVAVVSGAPLQGQLVARPSSINYMVAPVTGNDVGIRRAEIKQGIREVILCKDQDGKIGLRLKSIDNGIFVQLVQANSPASLVGLRFGDQVLQINGENCAGWSSDKAHKVLKQAFGEKITMTIRDRPFERTITMHKDSTGHVGFIFKNGKITSIVKDSSAARNGLLTEHNICEINGQNVIGLKDSQIADILSTSGTVVTITIMPAFIFEHIIKRMAPSIMKSLMDHTIPEV

SEQ ID NO: 20 - protein, 359 aa, Homo sapiens
MMLSLNNLQNIIYNPVIPFVGTIPDQLDPGTLIVIRGHVPSDADRFQVDLQNGSSMKPRADVAFHFNPRFKRAGCIVCNTLINEKWGREEITYDTPFKREKSFEIVIMVLKDKFQVAVNGKHTLLYGHRIGPEKIDTLGIYGKVNIHSIGFSFSSDLQSTQASSLELTEISRENVPKSGTPQLPSNRGGDISKIAPRTVYTKSKDSTVNHTLTCTKIPPMNYVSKRLPFAARLNTPMGPGRTVVVKGEVNANAKSFNVDLLAGKSKDIALHLNPRLNIKAFVRNSFLQESWGEEERNITSFPFSPGMYFEMIIYCDVREFKVAVNGVHSLEYKHRFKELSSIDTLEINGDIHLLEVRSW

SEQ ID NO: 21 - protein, 226 aa, Homo sapiens
MADPQAGSAAGDWEIDVESLELEEDVCGAPRSTPPGPSPPPADGDCEDDEDDDGVDEDAEEEGDGEEAGASPGMPGQPEQRGGPQPRPPLAPQASPAGTGPRERCTPAGGGAEPRKLSRTPKCARCRNHGVVSCLKGHKRFCRWRDCQCANCLLVVERQRVMAAQVALRRQQATEDKKGLSGKQNNFERKAVYQRQVRAPSLLAKSILEVLLGLFYSYYVYIMNHL

SEQ ID NO: 22 - protein, 8 aa, Homo sapiens
AVLRVARR

SEQ ID NO: 23 - protein, 7 aa, Homo sapiens
GAPILLR

SEQ ID NO: 24 - protein, 13 aa, Homo sapiens
MFEPVSCTFTYLL

SEQ ID NO: 25 - protein, 11 aa, Homo sapiens
REAVLIDPVLE

SEQ ID NO: 26 - protein, 11 aa, Homo sapiens
HTPGCVTFVLN

SEQ ID NO: 27 - protein, 8 aa, Homo sapiens
TGDALLIR

SEQ ID NO: 28 - protein, 14 aa, Homo sapiens
NPRLTLSCEEFVKI

SEQ ID NO: 29 - protein, 14 aa, Homo sapiens
NLPKPQQIDFAVPA

SEQ ID NO: 30 - protein, 17 aa, Homo sapiens
PAVPQDLQLPPSQRAQS

SEQ ID NO: 31 - protein, 8 aa, Homo sapiens
LKEHLLRR

SEQ ID NO: 32 - protein, 9 aa, Homo sapiens
RDQRVVTSE

SEQ ID NO: 33 - protein, 8 aa, Homo sapiens
EGTKVLKL

SEQ ID NO: 34 - protein, 12 aa, Homo sapiens
NNTVVGKHCIPL

SEQ ID NO: 35 - protein, 8 aa, Homo sapiens
TNSTVVID

SEQ ID NO: 36 - protein, 8 aa, Homo sapiens
QTPHLLLT

SEQ ID NO: 37 - protein, 9 aa, Homo sapiens
QHMTLSQAF

SEQ ID NO: 38 - protein, 7 aa, Homo sapiens
KKPVLGS

SEQ ID NO: 39 - protein, 7 aa, Homo sapiens
RGQIVQS

SEQ ID NO: 40 - protein, 8 aa, Homo sapiens
RKPLQVKD

SEQ ID NO: 41 - protein, 9 aa, Homo sapiens
ATKKLSATI

SEQ ID NO: 42 - protein, 12 aa, Homo sapiens
KPQPVNTSSVTV

SEQ ID NO: 43 - protein, 7 aa, Homo sapiens
TTKFVST

SEQ ID NO: 44 - protein, 11 aa, Homo sapiens
NTQLVRPPIRS

SEQ ID NO: 45 - protein, 16 aa, Homo sapiens
EKELLQSKTALSSVKT

SEQ ID NO: 46 - protein, 11 aa, Homo sapiens
IASEVVARPAS

SEQ ID NO: 47 - protein, 8 aa, Homo sapiens
AGKAIVDS

SEQ ID NO: 48 - protein, 8 aa, Homo sapiens
PPNSVVTQ

SEQ ID NO: 49 - protein, 10 aa, Homo sapiens
PKEDILVTLN

SEQ ID NO: 50 - protein, 19 aa, Homo sapiens
DAKKLVKYWICLALIEPIT

SEQ ID NO: 51 - protein, 16 aa, Homo sapiens
IENIIAIYEKAILAGA

SEQ ID NO: 52 - protein, 7 aa, Homo sapiens
RHTIVDI

SEQ ID NO: 53 - protein, 8 aa, Homo sapiens
EVKEVSIE

SEQ ID NO: 54 - protein, 7 aa, Homo sapiens
TGVDVDP

SEQ ID NO: 55 - protein, 9 aa, Homo sapiens
LHRNLLFQD

SEQ ID NO: 56 - protein, 9 aa, Homo sapiens
FKELKFLTP

SEQ ID NO: 57 - protein, 13 aa, Homo sapiens
LKDHYPCVSSLEQ

SEQ ID NO: 58 - protein, 17 aa, Homo sapiens
TDAFVCRPNAALCRVYY

SEQ ID NO: 59 - protein, 10 aa, Homo sapiens
YRLKLYSLRW

SEQ ID NO: 60 - protein, 7 aa, Homo sapiens
SDHEYLY

SEQ ID NO: 61 - protein, 7 aa, Homo sapiens
NNILVFN

SEQ ID NO: 62 - protein, 7 aa, Homo sapiens
GNSSVFL

SEQ ID NO: 63 - protein, 15 aa, Homo sapiens
PDGQFILLEYNYVKQ

SEQ ID NO: 64 - protein, 13 aa, Homo sapiens
WVTWSPVGHKLAY

SEQ ID NO: 65 - protein, 16 aa, Homo sapiens
WNNDIYVKIEPNLPSY

SEQ ID NO: 66 - protein, 12 aa, Homo sapiens
YEEEVFSAYSAL

SEQ ID NO: 67 - protein, 7 aa, Homo sapiens
GTFLAYA

SEQ ID NO: 68 - protein, 7 aa, Homo sapiens
SLSSVTN

SEQ ID NO: 69 - protein, 8 aa, Homo sapiens
ERISLQWL

SEQ ID NO: 70 - protein, 11 aa, Homo sapiens
QNYSVMDICDY

SEQ ID NO: 71 - protein, 8 aa, Homo sapiens
RWNCLVAR

SEQ ID NO: 72 - protein, 8 aa, Homo sapiens
YRHICYFQ

SEQ ID NO: 73 - protein, 7 aa, Homo sapiens
DKKDCTF

SEQ ID NO: 74 - protein, 17 aa, Homo sapiens
EVIGIEALTSDYLYYIS

SEQ ID NO: 75 - protein, 7 aa, Homo sapiens
GLRVLED

SEQ ID NO: 76 - protein, 8 aa, Homo sapiens
MLQNVQMP

SEQ ID NO: 77 - protein, 8 aa, Homo sapiens
KKLDFIIL

SEQ ID NO: 78 - protein, 9 aa, Homo sapiens
WYQMILPPH

SEQ ID NO: 79 - protein, 7 aa, Homo sapiens
WATYLAS

SEQ ID NO: 80 - protein, 7 aa, Homo sapiens
ENIIVAS

SEQ ID NO: 81 - protein, 7 aa, Homo sapiens
YYDSVYT

SEQ ID NO: 82 - protein, 9 aa, Homo sapiens
KQVEYLLIH

SEQ ID NO: 83 - protein, 10 aa, Homo sapiens
HKSPADIVKN

SEQ ID NO: 84 - protein, 8 aa, Homo sapiens
ESMAVLEK

SEQ ID NO: 85 - protein, 11 aa, Homo sapiens
VSKNLVAMKEI

SEQ ID NO: 86 - protein, 11 aa, Homo sapiens
QTEAVAQLAQE

SEQ ID NO: 87 - protein, 17 aa, Homo sapiens
YNSGLLSTLVADLQLID

SEQ ID NO: 88 - protein, 8 aa, Homo sapiens
KDVAQIFN

SEQ ID NO: 89 - protein, 19 aa, Homo sapiens
PTVEYICTQQNILFMLLKG

SEQ ID NO: 90 - protein, 10 aa, Homo sapiens
QFYDFFRYVE

SEQ ID NO: 91 - protein, 10 aa, Homo sapiens
EYEKLLHSEN

SEQ ID NO: 92 - protein, 12 aa, Homo sapiens
QSLKLLGELLLD

SEQ ID NO: 93 - protein, 13 aa, Homo sapiens
QFEAFHVFKVFVA

SEQ ID NO: 94 - protein, 20 aa, Homo sapiens
TQPILDILLKNQAKLIEFLS

SEQ ID NO: 95 - protein, 8 aa, Homo sapiens
KTYLVKQI

SEQ ID NO: 96 - protein, 13 aa, Homo sapiens
RKELVSRLLHLHF

SEQ ID NO: 97 - protein, 10 aa, Homo sapiens
KEAAVRGVRQ

SEQ ID NO: 98 - protein, 15 aa, Homo sapiens
LRADVDQLEKVLPQL

SEQ ID NO: 99 - protein, 11 aa, Homo sapiens
SDPSVEPPLSQ

SEQ ID NO: 100 - protein, 11 aa, Homo sapiens
HSYTASYDIYD

SEQ ID NO: 101 - protein, 11 aa, Homo sapiens
PENNVLSPLPS

SEQ ID NO: 102 - protein, 10 aa, Homo sapiens
YGFRLGFLHS

SEQ ID NO: 103 - protein, 11 aa, Homo sapiens
TAKSVTCTYSP

SEQ ID NO: 104 - protein, 20 aa, Homo sapiens
LNKMFCQLAKTCPVQLWVDS

SEQ ID NO: 105 - protein, 8 aa, Homo sapiens
RAMAIYKQ

SEQ ID NO: 106 - protein, 14 aa, Homo sapiens
HMTEVVRRCPHHER

SEQ ID NO: 107 - protein, 11 aa, Homo sapiens
GLAPPQHLIRV

SEQ ID NO: 108 - protein, 8 aa, Homo sapiens
PILTIITL

SEQ ID NO: 109 - protein, 9 aa, Homo sapiens
FEVRVCACP

SEQ ID NO: 110 - protein, 7 aa, Homo sapiens
PHHELPP

SEQ ID NO: 111 - protein, 7 aa, Homo sapiens
DGEYFTL

SEQ ID NO: 112 - protein, 7 aa, Homo sapiens
AHSSHLK

SEQ ID NO: 113 - protein, 8 aa, Homo sapiens
YKLVVVGA

SEQ ID NO: 114 - protein, 19 aa, Homo sapiens
GVGKSALTIQLIQNHFVDE

SEQ ID NO: 115 - protein, 16 aa, Homo sapiens
YRKQVVIDGETCLLDI

SEQ ID NO: 116 - protein, 8 aa, Homo sapiens
EGFLCVFA

SEQ ID NO: 117 - protein, 7 aa, Homo sapiens
FEDIHQY

SEQ ID NO: 118 - protein, 16 aa, Homo sapiens
DVPMVLVGNKCDLAAR

SEQ ID NO: 119 - protein, 15 aa, Homo sapiens
QAQDLARSYGIPYIE

SEQ ID NO: 120 - protein, 11 aa, Homo sapiens
VEDAFYTLVRE

SEQ ID NO: 121 - protein, 10 aa, synthetic construct
  REGION 1..10: note = Description of Artificial Sequence: Synthetic peptide
  REGION 1..10: note = MISC_FEATURE - This sequence may encompass 5-10 residues
HHHHHHHHHH